(19) United States
(12) Patent Application Publication
(10) Pub. No.: US 2014/0309426 A1
(43) Pub. Date: Oct. 16, 2014

(54) PHARMACEUTICAL COMPOSITIONS FOR THE TREATMENT OF LEFT VENTRICULAR DIASTOLIC DYSFUNCTION COMPRISING AN APOLIPOPROTEIN PEPTIDE/PHOSPHOLIPID COMPLEX

(71) Applicant: Institut de Cardiologie de Montreal, Montreal (CA)

(72) Inventors: Jean-Claude TARDIF, Montreal (CA); David Busseuil, Montreal (CA); Eric Rhéaume, Montreal (CA)

(21) Appl. No.: 14/312,084

(22) Filed: Jun. 23, 2014

Related U.S. Application Data
(63) Continuation of application No. 13/812,376, filed on Feb. 20, 2013, now abandoned.
(60) Provisional application No. 61/344,458, filed on Jul. 28, 2010.

(51) Int. Cl. C07C 327/30; C07D 295/16; C07C 323/63; C07D 213/75; C07D 307/54

(52) U.S. Cl. CPC C07C 327/30; C07D 213/75; C07D 307/54; C07C 323/63; C07D 295/16
USPC 546/221; 558/257; 546/297; 549/496; 564/123

(57) ABSTRACT

The present invention features pharmaceutical compositions and methods of using pharmaceutical compositions for treating left ventricular diastolic dysfunction. In particular, the pharmaceutical compositions include an apolipoprotein complex comprising a lipid fraction and a protein fraction.

### CROSS-REFERENCE TO RELATED APPLICATION

This application claims the benefit of the filing date of U.S. Provisional Application No. 61/344,458, filed Jul. 28, 2010, which is hereby incorporated by reference in its entirety.

### BACKGROUND OF THE INVENTION

Current standard of care for left ventricular diastolic dysfunction (LVDD) is limited to elimination of fluid overload with diuretics and to the identification and treatment of contributing factors such as left ventricular hypertrophy and myocardial ischemia. The most common cause of left ventricular hypertrophy is arterial hypertension, and attention is therefore given to treatment and control of blood pressure in patients with diastolic dysfunction. The presence of myocardial ischemia is also investigated and treated in the relevant patients with anti-ischemic drugs or revascularization. In a small number of patients, medical and/or mechanical treatment of hypertrophic cardiomyopathy can also lead to an improvement of diastolic dysfunction. Finally, beta-blockers and non-dihydropyridine calcium channel blocker have been used for the treatment of diastolic dysfunction because they reduce heart rate (see below).

Limitations and problems with the standard of care include the paucity of well-conducted randomized clinical trials in the field of left ventricular diastolic dysfunction, as well as the absence of well-powered trials demonstrating benefits of therapies. Also, beta-blockers and calcium-channel blockers are sometimes used in patients with diastolic dysfunction to slow heart rate in the hope that giving more time to diastolic filling will have favourable effects, but there are no robust data from randomized trials supporting their use. Indeed, to date there has been no specific pharmacologic treatment that has been approved by the FDA or endorsed in the guidelines of major societies for improving outcomes in patients with diastolic dysfunction.

The diagnosis of left ventricular diastolic dysfunction is applied to a broad range of patients with variable pathophysiology ranging from primary myocardial disease to progressive renal failure. The pathophysiologic mechanisms responsible for the development of diastolic dysfunction and diastolic heart failure remain poorly understood, in part because of the heterogeneous nature of the disorder. Known etiologies for left ventricular diastolic dysfunction include but are not limited to arterial hypertension with or without left ventricular hypertrophy, hypertrophic cardiomyopathy, myocardial ischemia, aging, diabetes mellitus, restrictive cardiomyopathy, amyloidosis, and constrictive pericarditis. Of note, coronary artery disease (coronary atherosclerosis) has been shown to be present in less than half of patients (47%) with diastolic heart failure (also called heart failure with preserved left ventricular ejection fraction) and relief of myocardial ischemia with revascularization has been shown to improve diastolic dysfunction in selected patients.

There is a need in the art for specific and effective therapies for the treatment of left diastolic dysfunction.

### SUMMARY OF THE INVENTION

The present invention provides pharmaceutical compositions and methods of using the pharmaceutical compositions for treating LVDD wherein the pharmaceutical compositions include an apolipoprotein complex comprising a lipid fraction and a protein fraction.

In one embodiment, the invention provides an apolipoprotein complex for treating LVDD wherein the protein fraction comprises a protein selected from the group consisting of: human preproApoA-I (SEQ ID NO. 1), human proApoA-I (SEQ ID NO. 2) and mature human ApoA-1 (SEQ ID NO. 3).

In one embodiment, the invention provides an apolipoprotein complex for treating LVDD wherein the protein fraction comprises a protein selected from the group consisting of: a genetic variant of human preproApoA-I, human proApoA-I (SEQ ID NO. 2) and mature ApoA-I (SEQ ID NO. 3).

In another embodiment, the invention provides an apolipoprotein complex for treating LVDD wherein the protein fraction comprises a protein selected from the group consisting of: human Milano variant of preproApoA-I (SEQ ID NO. 4), and human Milano variant of proApoA-I (SEQ ID NO. 5).

In another embodiment, the invention provides an apolipoprotein complex for treating LVDD wherein the protein fraction comprises a protein selected from the group consisting of: human Paris variant of preproApoA-I (SEQ ID NO. 6), and human Paris variant of proApoA-I (SEQ ID NO. 7).

In another embodiment, the invention provides an apolipoprotein complex for treating LVDD wherein the protein fraction comprises a protein selected from the group consisting of: human Zaragoza variant of preproApoA-I (SEQ ID NO. 8), and human Zaragoza variant of proApoA-I (SEQ ID NO. 9).

In another embodiment, the invention provides an apolipoprotein complex for treating LVDD wherein the protein fraction comprises a protein selected from the group consisting of: mature human ApoA-I (SEQ ID NO. 3), mature human Paris variant of ApoA-I (SEQ ID NO. 10), mature human Milano variant of ApoA-I (SEQ ID NO. 11), and mature human Zaragoza variant of ApoA-I (SEQ ID NO. 12).

In another embodiment, the invention provides an apolipoprotein complex for treating LVDD wherein the lipid fraction comprises both negatively and positively charged phospholipid.

In another embodiment, the invention provides an apolipoprotein complex for treating LVDD wherein the protein fraction comprises mature human ApoA-I (SEQ ID NO. 3) and the lipid fraction comprises negatively charged phosphatidylglycerol.

In another embodiment, the invention provides an apolipoprotein complex for treating LVDD wherein the protein fraction comprises mature human ApoA-I (SEQ ID NO. 3) and the lipid fraction comprises negatively charged phosphatidylglycerol wherein the molar ratio of the lipid fraction to the protein fraction is in the range of about 200:1 to 100:1.

In another embodiment, the invention provides an apolipoprotein complex for treating LVDD wherein the protein fraction comprises mature human ApoA-I (SEQ ID NO. 3) and the lipid fraction comprises negatively charged phosphatidylglycerol wherein the molar ratio of the lipid fraction to the protein is in the range of about 100:1 to 30:1.

In another embodiment, the invention provides an apolipoprotein complex for treating LVDD wherein the protein fraction comprises mature human ApoA-I (SEQ ID NO. 3) and the lipid fraction comprises negatively charged phosphatidylglycerol and the molar ratio of the lipid fraction to the protein is in the range of about 200:1 to 100:1.

In another embodiment, the invention provides an apolipoprotein complex for treating LVDD wherein the protein fraction comprises mature human ApoA-I (SEQ ID NO. 3) and the lipid fraction comprises sphingomyelin.

In another embodiment, the invention provides an apolipoprotein complex for treating LVDD wherein the protein fraction comprises mature human ApoA-I (SEQ ID NO. 3) and the lipid fraction comprises sphingomyelin and negatively charged phosphatidylglycerol.

In another embodiment, the invention provides an apolipoprotein complex for treating LVDD wherein the protein fraction comprises mature human ApoA-I (SEQ ID NO. 3) and the lipid fraction comprises sphingomyelin and negatively charged phosphatidylglycerol and the molar ratio of the lipid fraction to the protein fraction is in the range of about 100:1 to 30:1.

In one embodiment, the pharmaceutical composition for treating LVDD further comprises a pharmaceutically acceptable carrier, diluent and/or excipient.

In one embodiment, the invention provides an apolipoprotein complex for treating LVDD wherein the protein fraction comprises an ApoA-I analogue peptide.

In another embodiment, the invention provides an apolipoprotein complex for treating LVDD wherein the protein fraction comprises a 15-29 amino acid peptide that forms an amphipathic α-helix in the presence of lipids.

In another embodiment, the invention provides an apolipoprotein complex for treating LVDD wherein the protein fraction comprises a 15-29 amino acid peptide that forms an amphipathic α-helix in the presence of lipids and comprises a sequence according to Formula 1:

Z₁-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-Z₂₄ Formula 1

wherein
X₁ is Pro (P), Ala (A), Gly (G), Gln (Q), Asn (N), Asp (D) or D-Pro (p); X₂ is an aliphatic residue; X₃ is Leu (L) or Phe (F); X₄ is an acidic residue; X₅ is Leu (L) or Phe (F); X₆ is Leu (L) or Phe (F); X₇ is a hydrophilic residue; X₈ is an acidic or a basic residue; X₉ is Leu (L) or Gly (G); X₁₀ is Leu (L), Trp (W) or Gly (G); X₁₁ is a hydrophilic residue; X₁₂ is a hydrophilic residue; X₁₃ is Gly (G) or an aliphatic residue; X₁₄ is Leu (L), Trp (W), Gly (G) or Nal; X₁₅ is a hydrophilic residue; X₁₆ is a hydrophobic residue; X₁₇ is a hydrophobic residue; X₁₈ is Gln (Q), Asn (N) or a basic residue; X₁₉ is Gln (Q), Asn (N) or a basic residue; X₂₀ is a basic residue; X₂₁ is an aliphatic residue; X₂₂ is a basic residue; X₂₃ is absent or a basic residue; Z₁ is H₂N— or RC(O)NH—; and Z₂ is —C(O)NRR, —C(O)OR or —C(O)OH or a salt thereof;
R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and
each “-” between residues X₁ through X₂₃ designates an amide linkage, a substituted amide linkage, an isostere of an amide or an amide mimetic.

In another embodiment, the invention provides an apolipoprotein complex for treating LVDD wherein the protein fraction comprises a 22 to 29 amino acid peptide comprising a peptide selected from the group consisting of: SEQ ID NO. 54-101.

In another embodiment, the invention provides an apolipoprotein complex for treating LVDD wherein the protein fraction comprises a peptide and the peptide is N-terminal acylated, C-terminal amidated or esterified. In various embodiments, the peptide is any of the peptides described herein.

In another embodiment, the invention provides an apolipoprotein complex for treating LVDD wherein the protein fraction comprises a peptide selected from the group consisting of: SEQ ID NO. 54-101, including N-terminal acylated, C-terminal amidated and esterified forms thereof.

In another embodiment, the invention provides an apolipoprotein complex for treating LVDD wherein the protein fraction comprises a peptide of SEQ ID NO. 56.

In another embodiment, the invention provides an apolipoprotein complex for treating LVDD wherein the protein fraction comprises a 15-29 amino acid peptide that forms an amphipathic α-helix in the presence of lipids and comprises a sequence according to Formula 2:

R¹-Y¹-X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-Y²-R² (Formula 2)

wherein
X¹ is absent or a basic achiral amino acid residue, a basic D-amino acid residue, or a basic L-amino acid residue; X² is a basic achiral amino acid residue, a basic D-amino acid residue, or a basic L-amino acid residue; X³ is an aliphatic achiral amino acid residue, an aliphatic D-amino acid residue, or an aliphatic L-amino acid residue; X⁴ is a basic achiral amino acid residue, a basic D-amino acid residue, or a basic L-amino acid residue; X⁵ is Gln, Asn, D-Gln, D-Asn, or a basic achiral amino acid residue, a basic D-amino acid residue, or a basic L-amino acid residue; X⁶ is a basic a chiral amino acid residue, a basic D-amino acid residue, or a basic L-amino acid residue; X⁷ is a hydrophobic achiral amino acid residue, a hydrophobic D-amino acid residue, or a hydrophobic L-amino acid residue; X⁸ is a hydrophobic achiral amino acid residue, a hydrophobic D-amino acid residue, or a hydrophobic L-amino acid residue; X⁹ is a hydrophilic achiral amino acid residue, a hydrophilic D-amino acid residue, or a hydrophilic L-amino acid residue; X¹⁰ is Leu, Trp, Gly, Nal, D-Leu, D-Trp, or D-Nal; X¹¹ is Gly or an aliphatic achiral amino acid residue, an aliphatic D-amino acid residue, or an aliphatic L-amino acid residue; X¹² is a hydrophilic achiral amino acid residue, a hydrophilic D-amino acid residue, or a hydrophilic L-amino acid residue; X¹³ is a hydrophilic achiral amino acid residue, a hydrophilic D-amino acid residue, or a hydrophilic L-amino acid residue; X¹⁴ is Leu, Trp, Gly, D-Leu, or D-Trp; X¹⁵ is Leu, Gly, or D-Leu; X¹⁶ is an acidic achiral amino acid residue, an acidic D-amino acid residue, or an acidic L-amino acid residue; X¹⁷ is a hydrophilic achiral amino acid residue, a hydrophilic D-amino acid residue, or a hydrophilic L-amino acid residue; X¹⁸ is Leu, Phe, D-Leu, or D-Phe; X¹⁹ is Leu, Phe, D-Leu, or D-Phe; X²⁰ is an acidic achiral amino acid residue, an acidic D-amino acid residue, or an acidic L-amino acid residue; X²¹ is Leu, Phe, D-Leu, or D-Phe; X²² is an aliphatic achiral amino acid residue, an aliphatic D-amino acid residue, or an aliphatic L-amino acid residue; and X²³ is Inp, Nip, azPro, Pip, azPip, D-Nip, or D-Pip;
Y¹ is absent or a sequence of 1 to 7 amino acid residues, wherein each residue of the sequence is independently an achiral, D-, or L-amino acid residue;
Y² is absent or a sequence of 1 to 7 amino acid residues, wherein each residue of the sequence is independently an achiral, D-, or L-amino acid residue;
R¹ is H or an amino protecting group; and R² is OH or a carboxyl protecting group; and wherein: (a) all amino acid residues, other than the terminal amino acid residues and residues immediately adjacent to the terminal amino acid residues, are achiral or L-amino acid residues; or (b) all amino acid residues, other than the terminal amino acid residues and residues immediately adjacent to the terminal amino acid residues, are achiral or D-amino acid residues.

In another embodiment, the invention provides an apolipoprotein complex for treating LVDD wherein the protein fraction comprises a 22 to 29 amino acid peptide comprising a peptide selected from the group consisting of: SEQ ID NO. 102 to 165.

In another embodiment, the invention provides an apolipoprotein complex for treating LVDD wherein the protein fraction comprises a peptide selected from the group consisting of: SEQ ID NO. 102 to 165.

In another embodiment, the invention provides an apolipoprotein complex for treating LVDD wherein the protein fraction comprises the peptide of SEQ ID NO. 116.

In one embodiment, the apolipoprotein complex for use in the invention comprising the peptide of SEQ ID NO. 116 and sphingomyelin (SPH), 1,2-dipalmitoyl-sn-glycero-3-phosphocholine (DPPC) and 1,2-dipalmitoyl-sn-glycero-3-[phospho-rac-(1-glycerol)] (DPPG) in the lipid fraction.

In a further embodiment, the apolipoprotein complex has a ratio of peptide to phospholipid of 1/2.5 and a lipid composition of 48.5% SPH/48.5% DPPC/3% DPPG (w/w/w).

The present invention provides a CETP inhibitor for the treatment of LVDD.

In one embodiment, Dalcetrapib (Propanethioic acid, 2-methyl-, S-[2-[[[1-(2-ethylbutyl)cyclohexyl]carbonyl]amino]phenyl]ester) or a pro-drug compound, a pharmaceutically acceptable salt, hydrate, or solvate thereof is used for the treatment of LVDD.

In another embodiment, a compound selected from the group consisting of:
S-[2-(1-isopentylcyclohexanecarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxy-thioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)-phenyl]2,2-dimethylthiopropionate;
S-[4,5-dichloro-2-(1-isopentylcyclopentanecarbonylamino)-phenyl]2,2-dimethylthiopropionate;
S-[2-(1-isopentylcyclohexanecarbonylamino)-4-trifluoromethylphenyl]2,2-dimethylthiopropionate;
O-methyl S-[2-(1-isopentylcyclohexanecarbonylamino phenyl monothiocarbonate;
S-[2-(1-methylcyclohexanecarbonylamino)phenyll]S-phenyldithiocarbonate;
S-[2-(1-isopentylcyclohexanecarbonylamino)phenyl]N-phenylthiocarbamate;
S-[2-(pivaloylamino)-4-trifluoromethylphenyl]2,2-dimethylthiopropionate;
S-[4,5-dichloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate;
S-[4,5-dichloro-2-(1-pentylcyclohexanecarbonylamino)-phenyl]2,2-dimethylthiopropionate;
S-[4,5-dichloro-2-(1-cyclopropylmethylcyclohexanecarbonylamino)phenyl]2,2-dimethylthiopropionate;
S-[4,5-dichloro-2-(1-cyclohexylmethylcyclohexanecarbonylamino)phenyl]2,2-dimethylthiopropionate;
S-[4,5-dichloro-2-(1-isopropylcyclohexanecarbonylamino)-phenyl]2,2-dimethylthiopropionate;
S-[4,5-dichloro-2-(1-isopentylcycloheptanecarbonylamino)-phenyl]2,2-dimethylthiopropionate;
S-[4,5-dichloro-2-(1-isopentylcyclobutanecarbonylamino)-phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dim ethylthiopropionate;
S-[5-chloro-2-(1-isopentylcyclohexanecarbonylamino)phenyl]2,2-dimethylthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)-phenyl]2,2-dimethylthiopropionate;
S-[5-fluoro-2-(1-isopentylcyclohexanecarbonylamino)phenyl]2,2-dimethylthiopropionate;
bis-[4,5-dichloro-2-(1-isopentylcyclohexanecarbonylamino)-phenyl]disulfide;
2-tetrahydrofurylmethyl 2-(1-isopentylcyclohexanecarbonylamino) phenyl disulfide;
N-(2-mercaptophenyl)-1-ethylcyclohexanecarboxamide;
N-(2-mercaptophenyl)-1-propylcyclohexanecarboxamide;
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyll]cyclohexanethiocarboxylate;
S-[2-(1-isopentylcyclohexanecarbonylamino)phenyll]thiobenzoate;
S-[2-(1-isopentylcyclohexanecarbonylamino)phenyll]5-carboxythiopentanoate;
S-[2-(1-isopentylcyclohexanecarbonylamino)-4-methylphenyl]thioacetate;
bis-[2-[1-(2-ethylbutyl)cyclohexanecarbonylamino]phenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methylthiopropionate;
S-[2-(1-isobutylcyclohexanecarbonylamino)phenyl]2-methylthiopropionate;
S-[2-[1-(2-ethylbutyl)cyclohexanecarbonylamino]phenyl]1-acetylpiperidine-4-thiocarboxylate;
S-[2-[1-(2-ethylbutyl)cyclohexanecarbonylamino]phenyl]thioacetate;
S-[2-[1-(2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-1-(2-ethylbutyl)cyclohexanecarbonylamino]phenyl]2-hydroxy-2-methylthiopropionate;
S-[2-[1-(2-ethylbutyl)cyclohexanecarbonylamino]phenyl]4-chlorophenoxythioacetate;
S-[2-(1-isobutylcyclohexanecarbonylamino)phenyl]4-chlorophenoxythioacetate; and
S-[2-(1-isobutylcyclohexanecarbonylamino)phenyl]-1-acetyl-piperidine-4-thiocarboxylate, or a pro-drug compound, a pharmaceutically acceptable salt, hydrate, or solvate thereof is used for the treatment of LVDD.

In one embodiment, Anacetrapib ((4S,5R)-5-[3,5-bis(trifluoromethyl)phenyl]-3-{[4′-fluoro-2′-methoxy-5′-(propan-2-yl)-4-(trifluoromethyl)[1,1′-biphenyl]-2-yl]methyl}-4-methyl-I,3-oxazolidin-2-one) or a pro-drug compound, a pharmaceutically acceptable salt, hydrate, or solvate thereof is used for the treatment of LVDD.

In another embodiment, a compound according to Formula 5, wherein:

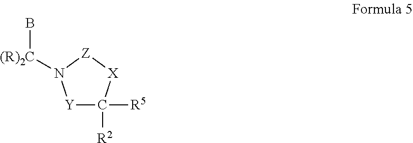

### Y is —(CRR¹)— or —C(═O)—

X is selected from the group consisting of —O—, —NH—, —N(C₁-C₅alkyl)-, and (CRR⁶)—
Z is selected from the group consisting of —C(═O)—, —S(O)₂—, and —C(═N—R⁹)—, wherein R⁹ is selected from the group consisting of H, —CN, and CH₃;
each R is independently selected from the group consisting of H and C₁-C₅alkyl and halogen, wherein C₁-C₅ alkyl is optionally substituted with 1-11 halogens;
B is selected from the group consisting of A¹ and A², wherein A¹ has the structure:

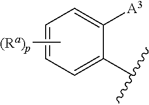

R¹ and R⁶ are each selected from the group consisting of H, —C₁-C₅ alkyl, halogen, and —(C(R)₂)ₙA², wherein —C₁-C₅ alkyl is optionally substituted with 1-11 halogens;
R² is selected from the group consisting of H, —C₁-C₅ alkyl, and —(C(R)₂)ₙA², wherein —C₁-C₅ alkyl is optionally substituted with 1-11 halogens;
wherein one of B and R² is A¹; and one of B, R¹, and R² is A² or —(C(R)₂)ₙA²; so that the compound of Formula I comprises one group A¹ and one group A²;
A³ is selected from the group consisting of: (a) an aromatic ring selected from phenyl and napthyl; (b) a 5-6-membered non-aromatic cycloalkyl ring, which optionally comprises 1-2 heteroatoms independently selected from N, S, O, and —N(O)—, wherein the point of attachment of A³ to the phenyl ring to which A³ is attached is a carbon atom; and (c) a benzoheterocyclic ring comprising a phenyl ring fused to a 5-membered aromatic heterocyclic ring having 1-2 heteroatoms independently selected from 0, N, and —S(O)ₓ, wherein the point of attachment of A₃ to the phenyl ring to which A³ is attached is a carbon atom;
A² is selected from the group consisting of: (a) an aromatic ring selected from phenyl and napthyl; (b) a benzoheterocyclic ring comprising a phenyl ring fused to a 5-membered aromatic heterocyclic ring having 1-2 heteroatoms independently selected from 0, N, and —S; (c) a 5-6-membered heterocyclic ring having 1-4 heteroatoms independently selected from N, S, O, and —N(O)—, and optionally also comprising 1-3 double bonds; (d) a benzoheterocyclic ring comprising a phenyl ring fused to a 5-membered heterocyclic ring having 1-2 heteroatoms independently selected from O, N, and S; and (e) a —C₅-C₆ cycloalkyl ring optionally having 1-3 double bonds;
each R^{a} is independently selected from the group consisting of —C₁-C₆ alkyl, —C₂-C₆ alkenyl, —C₂-C₆ alkynyl, —C₃-C₈ cycloalkyl optionally having 1-3 double bonds, —OC₁-C₆ alkyl, —OC₂-C₆ alkenyl, —OC₂-C₆ alkynyl, —OC₃-C₈ cycloalkyl optionally having 1-3 double bonds, —C(═O)C₁-C₆ alkyl, —C(═O)C₃-C₈ cycloalkyl, —C(═O)OH, —CO₂H, —CO₂C₁-C₆ alkyl, —C(═O)SC₁-C₆ alkyl, —OH, —NR³R⁴, —C(═O)NR³R⁴, —NR³C(═O)OC₁-C₆ alkyl, —NR³C(═O)NR³R⁴, —S(O)—C₁-C₆ alkyl, —S(O)_{y}NR³R⁴, —NR³S(O)_{y}NR³R⁴, halogen, —CN, —NO₂, and a 5-6-membered heterocyclic ring having 1-4 heteroatoms independently selected from N, S, and O, said heterocyclic ring optionally also comprising a carbonyl group and optionally also comprising 1-3 double bonds, wherein the point of attachment of said heterocyclic ring to the ring to which R^{a} is attached is a carbon atom, wherein said heterocyclic ring is optionally substituted with 1-5 substituent groups independently selected from halogen, —C₁-C₃ alkyl, and —OC₁-C₃ alkyl, wherein —C₁-C₃ alkyl and —OC₁-C₃ alkyl are optionally substituted with 1-7 halogens; wherein for compounds in which R^{a} is selected from the group consisting of —C₁-C₆ alkyl, —C₂-C₆ alkenyl, —C₂-C₆ alkynyl, —C₃-C₈ cycloalkyl optionally having 1-3 double bonds, —OC₁-C₆ alkyl, —OC₂-C₆ alkenyl, —OC₂-C₆alkynyl, —OC₃-C₈ cycloalkyl optionally having 1-3 double bonds, —C(═O)C₁-C₆ alkyl, —C(═O)C₃-C₈ cycloalkyl, —CO₂C₁-C₆ alkyl, —C(═O)SC₁-C₆ alkyl, —NR₃C(═O)OC₁-C₆ alkyl, and —S(O)ₓC₁-C₆ alkyl, R^{a} is optionally substituted with 1-15 halogens and is optionally also substituted with 1-3 substituent groups independently selected from (a) —OH, (b) —CN, (c) —NR₃R₄, (d) —C₃-C₈ cycloalkyl optionally having 1-3 double bonds and optionally substituted with 1-15 halogens, (e) —OC₁-C₄alkyl optionally substituted with 1-9 halogens and optionally also substituted with 1-2 substituent groups independently selected from —OC₁-C₂ alkyl and phenyl, (f) —OC₃-C₈ cycloalkyl optionally having 1-3 double bonds and optionally substituted with 1-15 halogens, (g) —CO₂H, (h) —C(═O)CH₃, (i) —CO₂C₁-C₄ alkyl which is optionally substituted with 1-9 halogens, and (j) phenyl which is optionally substituted with 1-3 groups independently selected from halogen, —CH₃, —CF₃, —OCH₃, and —OCF₃; with the proviso that when B is A¹, and X and Y are —CH₂—, and Z is —C(═O)—, and R² is phenyl which has a substituent R^{a} in the 4-position, wherein R^{a} is —OC₁-C₆alkyl which is optionally substituted as described above, then there are no other R^{a} substitutents on R² in which R^{a} is selected from —OH, —OC₁-C₆alkyl, —OC₂-C₆ alkenyl, —OC₂-C₆ alkynyl, and —OC₃-C₆ cycloalkyl optionally having 1-3 double bonds, optionally substituted as described above;
n is 0 or 1;
p is an integer from 0-4;
x is 0, 1, or 2;
y is 1 or 2;

R³ and R⁴ are each independently selected from H, —C₁-C₅ alkyl, —C(═O)C₁-C₅ alkyl and —S(O)_{y}C₁-C₅ alkyl, wherein —C₁-C₅ alkyl in all instances is optionally substituted with 1-11 halogens; and R⁵ is selected from the group consisting of H, —OH, —C₁-C₅ alkyl, and halogen, wherein —C₁-C₅ alkyl is optionally substituted with 1-11 halogens;

is used for the treatment of LVDD.

In one embodiment, a compound according to Formula 6, wherein:

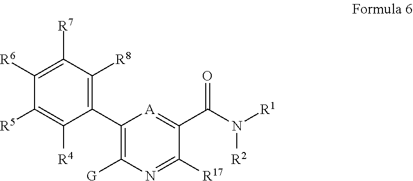

### A is CH;

R² is hydrogen and R′ is selected from the group consisting of: (a) cycloalkyl, which is optionally substituted by hydroxy, lower hydroxyalkyl or lower alkoxy, (b) 1-hydroxy-2-indanyl, (c) lower hydroxyalkyl, (d) lower hydroxyhalogenalkyl, (e) lower hydroxyalkoxyalkyl, (f) —CH₂—CR⁹R¹⁰-cycloalkyl, wherein R⁹ is hydrogen or lower alkyl; and wherein R¹⁰ is hydrogen, hydroxy or lower alkoxy; and (g) —CR¹¹R¹²—COOR¹³; wherein R¹¹ and R¹² independently from each other are hydrogen or lower alkyl; and wherein R¹³ is lower alkyl; or alternatively, R¹ and R² together with the nitrogen atom to which they are attached form a morpholinyl ring;
G is a group selected from the group consisting of: (a) —X—R³, wherein X is O or NR¹⁴, wherein R¹⁴ is selected from the group consisting of hydrogen, lower alkyl and lower hydroxyalkyl; and R³ is lower cycloalkylalkyl, (b) —C═C—R¹⁵, wherein R¹⁵ is selected from the group consisting of lower alkoxyalkyl, cycloalkyl and furanyl substituted by halogen; and (c) —CH₂—CH₂—R¹⁶,
wherein R¹⁶ is selected from the group consisting of: (1) a cycloalkyl which is optionally substituted by hydroxy or lower alkoxy, (2) a heteroaryl which is pyridyl or imidazolyl, which is optionally substituted by lower alkyl or halogen, and (3) lower alkylaminocarbonyl or lower alkylcarbonylamino;
R⁴ and R⁸ independently from each other are hydrogen or halogen;
R⁵ and R⁷ independently from each other are selected from the group consisting of hydrogen, lower alkyl, lower alkoxy, halogen, lower halogenalkyl, lower halogenalkoxy and cyano;
R⁶ is selected from the group consisting of hydrogen, lower alkoxy, halogen, lower halogenalkyl, lower halogenalkoxy and cyano; and
R¹⁷ is a lower halogenalkyl,
is used for the treatment of LVDD.

In one embodiment, 5-(4-chloro-phenyl)-6-cyclopropylmethoxy-N-((1R,2R)-2-hydroxy-cyclohexyl)-2-trifluoromethyl-nicotinamide or a pharmaceutically acceptable salt thereof is used for the treatment of LVDD.

In one embodiment, a compound according to Formula 7, wherein:

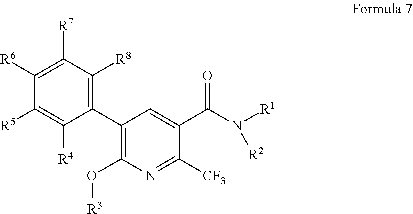

R¹ is selected from the group consisting of: (1) lower hydroxyalkyl, (2) cycloalkyl which is unsubstituted or substituted by hydroxy or lower hydroxyalkyl, and (3) —CH₂—CR⁹R¹⁰-cycloalkyl, wherein R⁹ is hydrogen or lower alkyl, and R¹⁰ is hydrogen or hydroxy;
R² is hydrogen;
R³ is selected from the group consisting of: (1) lower alkoxyalkyl, (2) lower halogenalkyl, and (3) lower heteroarylalkyl, wherein the heteroaryl group is unsubstituted or substituted once or twice by lower alkyl;
R⁴ and R⁸ are hydrogen; and
R⁵, R⁶ and R⁷ independently from each other are selected from the group consisting of: (1) hydrogen, (2) lower alkyl, (3) halogen, (4) lower halogenalkyl, (5) lower halogenalkoxy, (6) lower alkylsulfonylamino, and (7) cyano,
is used for the treatment of LVDD.

In another embodiment, a compound selected from the group consisting of:
5-(4-chloro-phenyl)-N-(2-cyclopropyl-2-hydroxy-propyl)-6-(2,2,2-trifluoro-ethoxy)-2-trifluoromethyl-nicotinamide,
N-(2-cyclopropyl-2-hydroxy-propyl)-5-(3,4-dichloro-phenyl)-6-(2,2,2-trifluoro-ethoxy)-2-trifluoromethyl-nicotinamide,
N—((R)-2-cyclopropyl-2-hydroxy-propyl)-5-(3,4-dichloro-phenyl)-6-(2,2,2-trifluoroethoxy)-2-trifluoromethyl-nicotinamide
N—((S)-2-cyclopropyl-2-hydroxy-propyl)-5-(3,4-dichloro-phenyl)-6-(2,2,2-trifluoroethoxy)-2-trifluoromethyl-nicotinamide
5-(3-chloro-phenyl)-N-(2-cyclopropyl-2-hydroxy-propyl)-6-(2,2,2-trifluoro-ethoxy)-2-trifluoromethyl-nicotinamide,
5-(4-chloro-phenyl)-N-((1R,2R)-2-hydroxy-cyclohexyl)-6-(2,2,2-trifluoro-ethoxy)-2-trifluoromethyl-nicotinamide,
5-(4-chloro-phenyl)-N-((1R,2S)-2-hydroxy-cyclohexyl)-6-(2,2,2-trifluoro-ethoxy)-2-trifluoromethyl-nicotinamide,
5-(4-chloro-phenyl)-N-((1S,2R)-2-hydroxy-cyclohexyl)-6-(2,2,2-trifluoro-ethoxy)-2-trifluoromethyl-nicotinamide,
5-(4-chloro-phenyl)-N-((1S,2S)-2-hydroxy-cyclohexyl)-6-(2,2,2-trifluoro-ethoxy)-2-trifluoromethyl-nicotinamide,
N-(1-hydroxymethyl-3-methyl-butyl)-5-(4-methanesulfonylamino-phenyl)-6-(pyridin-2-ylmethoxy)-2-trifluoromethyl-nicotinamide,
N-((1R,2R)-2-hydroxy-cyclohexyl)-5-(4-methanesulfonylamino-phenyl)-6-(2,2,2-trifluoro-ethoxy)-2-trifluoromethyl-nicotinamide,
5-(3,4-dichloro-phenyl)-N-(1R,2R)-2-hydroxy-cyclohexyl)-6-(pyridin-2-ylmethoxy)-2-trifluoromethyl-nicotinamide, or a pharmaceutically acceptable salt or solvate thereof.
   is used for the treatment of LVDD.

In one embodiment, a compound according to Formula 8, wherein:

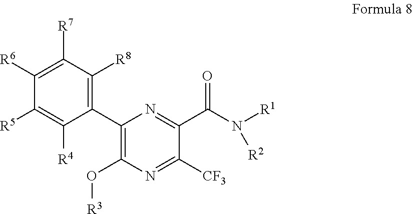

R¹ is selected from the group consisting of: (1) cycloalkyl, which is unsubstituted or substituted by hydroxy or lower hydroxyalkyl, and (2) —CH₂—CR⁹R¹⁰-cycloalkyl, wherein R⁹ is hydrogen or lower alkyl, and R¹⁰ is hydrogen or hydroxy;
R² is hydrogen;
R³ is selected from the group consisting of: (1) lower cycloalkylalkyl, (2) lower alkoxyalkyl, (3) lower halogenalkyl, (4) lower heteroarylalkyl, wherein the heteroaryl group is unsubstituted or substituted once or twice by lower alkyl, and (5) phenyl, which is unsubstituted or substituted once or twice by halogen;
R⁴ and R⁸ independently from each other are hydrogen or halogen; and
R⁵, R⁶ and R⁷ independently from each other are selected from the group consisting of: (1) hydrogen, (2) lower alkyl, (3) lower alkoxy, (4) halogen, (5) lower halogenalkyl, (6) lower halogenalkoxy, (7) lower alkylsulfonylamino, and (8) cyano.
is used for the treatment of LVDD.

In one embodiment, a compound selected from the group consisting of:
6-(4-chloro-phenyl)-5-(2,2,2-trifluoro-ethoxy)-3-trifluoromethyl-pyrazine-2-carboxylic acid (2-cyclopropyl-2-hydroxy-propyl)-amide,
6-(4-chloro-phenyl)-5-cyclopropylmethoxy-3-trifluoromethyl-pyrazine-2-carboxylic acid ((1R,2R)-2-hydroxy-cyclohexyl)-amide,
6-(4-chloro-phenyl)-5-cyclopropylmethoxy-3-trifluoromethyl-pyrazine-2-carboxylic acid (2-cyclopropyl-2-hydroxy-propyl)-amide,
6-(3-chloro-phenyl)-5-(2,2,2-trifluoro-ethoxy)-3-trifluoromethyl-pyrazine-2-carboxylic acid ((1R,2R)-2-hydroxy-cyclohexyl)-amide,
6-(3-chloro-phenyl)-5-(2,2,2-trifluoro-ethoxy)-3-trifluoromethyl-pyrazine-2-carboxylic acid (2-cyclopropyl-2-hydroxy-propyl)-amide,
6-(3-chloro-phenyl)-5-cyclopropylmethoxy-3-trifluoromethyl-pyrazine-2-carboxylic acid ((1R,2R)-2-hydroxy-cyclohexyl)-amide,
6-(3-chloro-phenyl)-5-cyclopropylmethoxy-3-trifluoromethyl-pyrazine-2-carboxylic acid (2-cyclopropyl-2-hydroxy-propyl)-amide,
6-(3-chloro-phenyl)-5-phenoxy-3-trifluoromethyl-pyrazine-2-carboxylic acid ((1R,2R)-2-hydroxy-cyclohexyl)-amide,
6-(3-chloro-phenyl)-5-phenoxy-3-trifluoromethyl-pyrazine-2-carboxylic acid (2-cyclopropyl-2-hydroxy-propyl)-amide,
6-(3-chloro-phenyl)-5-(pyridin-2-ylmethoxy)-3-trifluoromethyl-pyrazine-2-carboxylic acid ((1R,2R)-2-hydroxy-cyclohexyl)-amide,
6-(3-chloro-phenyl)-5-(pyridin-2-ylmethoxy)-3-trifluoromethyl-pyrazine-2-carboxylic acid (2-cyclopropyl-2-hydroxy-propyl)-amide,
6-(3,4-dichloro-phenyl)-5-(2,2,2-trifluoro-ethoxy)-3-trifluoromethyl-pyrazine-2-carboxylic acid ((1R,2R)-2-hydroxy-cyclohexyl)-amide,
6-(3,4-dichloro-phenyl)-5-(2,2,2-trifluoro-ethoxy)-3-trifluoromethyl-pyrazine-2-carboxylic acid (2-cyclopropyl-2-hydroxy-propyl)-amide,
6-(3,4-dichloro-phenyl)-5-cyclopropylmethoxy-3-trifluoromethyl-pyrazine-2-carboxylic acid ((1R,2R)-2-hydroxy-cyclohexyl)-amide,
6-(3,4-dichloro-phenyl)-5-cyclopropylmethoxy-3-trifluoromethyl-pyrazine-2-carboxylic acid (2-cyclopropyl-2-hydroxy-propyl)-amide,
6-(3,4-dichloro-phenyl)-5-phenoxy-3-trifluoromethyl-pyrazine-2-carboxylic acid (1R,2R)-2-hydroxy-cyclohexyl)-amide,
6-(3,4-dichloro-phenyl)-5-phenoxy-3-trifluoromethyl-pyrazine-2-carboxylic acid (2-cyclopropyl-2-hydroxy-propyl)-amide,
6-(3,4-dichloro-phenyl)-5-(pyridin-2-ylmethoxy)-3-trifluoromethyl-pyrazine-2-carboxylic acid ((1R,2R)-2-hydroxy-cyclohexyl)-amide,
6-(3,4-dichloro-phenyl)-5-(pyridin-2-ylmethoxy)-3-trifluoromethyl-pyrazine-2-carboxylic acid (2-cyclopropyl-2-hydroxy-propyl)-amide,
6-(4-methanesulfonylamino-phenyl)-5-(2,2,2-trifluoro-ethoxy)-3-trifluoromethyl-pyrazine-2-carboxylic acid (1R,2R)-2-hydroxy-cyclohexyl)-amide,
6-(4-methanesulfonylamino-phenyl)-5-(2,2,2-trifluoro-ethoxy)-3-trifluoromethyl-pyrazine-2-carboxylic acid (2-cyclopropyl-2-hydroxy-propyl)-amide,
6-(4-methanesulfonylamino-phenyl)-5-cyclopropylmethoxy-3-trifluoromethyl-pyrazine-2-carboxylic acid (1R,2R)-2-hydroxy-cyclohexyl)-amide,
6-(4-methanesulfonylamino-phenyl)-5-cyclopropylmethoxy-3-trifluoromethyl-pyrazine-2-carboxylic acid (2-cyclopropyl-2-hydroxy-propyl)-amide,
6-(4-methanesulfonylamino-phenyl)-5-phenoxy-3-trifluoromethyl-pyrazine-2-carboxcylic acid ((1R,2R)-2-hydroxy-cyclohexyl)-amide,
6-(4-methanesulfonylamino-phenyl)-5-phenoxy-3-trifluoromethyl-pyrazine-2-carboxylic acid (2-cyclopropyl-2-hydroxy-propyl)-amide,
6-(4-methanesulfonyl amino-phenyl)-5-(pyridin-2-ylmethoxy)-3-trifluoromethyl-pyrazine-2-carboxylic acid ((1R,2R)-2-hydroxy-cyclohexyl)-amide, and
6-(4-methanesulfonylamino-phenyl)-5-(pyridin-2-ylmethoxy)-3-trifluoromethyl-pyrazine-2-carboxylic acid (2-cyclopropyl-2-hydroxy-propyl)-amide,
   is used for the treatment of LVDD.

In one embodiment, a compound according to Formula 9, wherein:

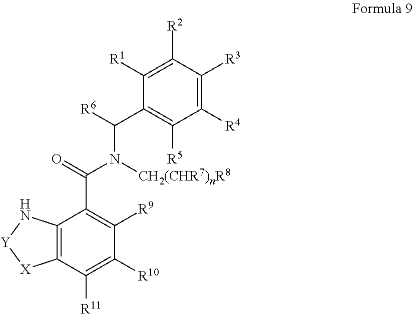

wherein —X—Y— is —CR═CR^{c}— or —CR^{a}═N— or —CR^{a}R^{b}—CR^{c}R^{d}—,
R^{a}, R^{b}, R^{c} and R^{d} are independently from each other selected from the group consisting of hydrogen and C₁-C₈ alkyl;
R¹, R², R⁴ and R⁵ are independently from each other selected from the group consisting of hydrogen, C₁-C₈ alkyl, C₁-C₈ alkoxy, halogen and halogen-C₁-C₈ alkyl;
R³ is Si(CH₃)₃ or Si(CH₃)₂CH(CH₃)₂;
R⁶ is selected from the group consisting of hydrogen and C₁-C₈ alkyl;
R⁷ is selected from the group consisting of hydrogen, C₁-C₈ alkyl, hydroxy and halogen;
R⁸ is selected from the group consisting of C₁-C₈ alkyl, C₂-C₈ alkenyl, halogen-C₁-C₈ alkyl, heterocyclyl, heteroaryl which is unsubstituted or substituted by one or two groups independently selected from C₁-C₈ alkyl, C₁-C₈ alkoxy, halogen-C₁-C₈ alkyl, halogen-C₁-C₈ alkoxy and halogen, phenyl which is unsubstituted or substituted by one or two groups independently selected from C₁-C₈ alkyl, C₁-C₈ alkoxy, halogen-C₁-C₈ alkyl, halogen-C₁-C₈ alkoxy and halogen, —OR₁₂, wherein R₁₂ is C₁-C₈ alkyl or phenyl which is unsubstituted or substituted by one or two groups independently selected from C₁-C₈ alkyl, C₁-C₈ alkoxy, halogen-C₁-C₈ alkyl, halogen-C₁-C₈ alkoxy and halogen, —NR¹³R¹⁴, wherein R¹³ and R¹⁴ independently from each other are selected from hydrogen, C₁-C₈ alkyl, and phenyl which is unsubstituted or substituted by one or two groups independently selected from C₁-C₈ alkyl, C₁-C₈ alkoxy, halogen-C₁-C₈ alkyl, halogen-C₁-C₈ alkoxy and halogen, and —C(O)—OR¹⁵, wherein R¹⁵ is hydrogen or C₁-C₈ alkyl; R⁹, R¹⁰ and R¹¹ independently from each other are selected from the group consisting of hydrogen, C₁-C₈ alkyl, cycloalkyl, C₁-C₈ alkoxy, halogen-C₁-C₅ alkyl, and halogen; and n is 1, 2 or 3.

In particular embodiments, compounds of Formula 9 wherein: R⁸ is heterocyclyl or heteroaryl which is unsubstituted or substituted by one or two groups independently selected from C₁-C₈ alkyl, C₁-C₈ alkoxy, halogen-C₁-C₅ alkyl, halogen-C₁-C₈ alkoxy and halogen; or

R⁸ is —OR¹², and R¹² is C₁-C₈ alkyl or phenyl which is unsubstituted or substituted by one or two groups independently selected from C₁-C₅ alkyl, C₁-C₈ alkoxy, halogen-C₁-C₈ alkyl, halogen-C₁-C₈ alkoxy and halogen; or
R⁸ is —NR¹³R¹⁴, wherein R¹³ and R¹⁴ independently from each other are selected from hydrogen, C₁-C₈ alkyl, and phenyl which is unsubstituted or substituted by one or two groups independently selected from C₁-C₈ alkyl, C₁-C₈ alkoxy, halogen-C₁-C₈ alkyl, halogen-C₁-C₈ alkoxy and halogen;
or
R⁸ is C(O)—OR¹⁵, wherein R¹⁵ is hydrogen or C₁-C₅ alkyl; or
R⁸ is phenyl which is unsubstituted or substituted by one oar two groups independently selected from C₁-C₈ alkyl, C₁-C₅ alkoxy, halogen-C₁-C₈ alkyl, halogen-C₁-C₈ alkoxy and halogen.

For any of the compounds and methods described herein, the present invention also relates to pharmaceutically acceptable salts, hydrates, or solvates of any compound described herein (e.g., any compounds of formulas 4-9) for the treatment of LVDD. In particular embodiments, the compounds for the treatment of LVDD is a compound of any one of formula 4-9, or a pharmaceutically acceptable salt thereof

The present invention also provides an ABCA1 agonist for the treatment of LVDD.

The present invention also provides an anti-microRNA-33 (anti-miR-33) compound, e.g., any described herein, for the treatment of LVDD.

### BRIEF DESCRIPTION OF THE FIGURES

FIG. 1: illustrates the effect of the ALPC-I A treatment by comparing the distribution of diastolic dysfunction severity in control (upper panel) and treated (in lower panel) subjects (n=6 in each group) as a function of time. At the end of the two weeks treatment, left ventricular diastolic filling patterns were distributed differently among groups (P=0.018)

FIG. 2: illustrates the effect of the ALPC-2 treatment by comparing the distribution of diastolic dysfunction severity in control (upper panel) and treated (lower panels) subjects (n=12 in each group) as a function of time. These results show decreased severity of diastolic dysfunction in the treated groups which reach statistical significance at day 14 after initiation of treatment (p=0.048).

### DETAILED DESCRIPTION OF THE INVENTION

### I. Definitions

Unless otherwise indicated, the following definitions are set forth to illustrate and define the meaning and scope of the various terms used to describe the invention herein.

“Left ventricular diastolic dysfunction” or “LVDD” as used herein mean an abnormality in the filling of the left ventricle of the heart during diastole; the phase of the cardiac cycle when the muscle of the left ventricle is relaxed and filling with blood that is being returned to the heart from the lungs. As used herein the terms diastolic dysfunction or ventricular diastolic dysfunction do not include right ventricular diastolic dysfunction. Ventricular diastolic function is associated with the following conditions. The present invention provides pharmaceutical compositions for the treatment of ventricular diastolic dysfunction.

“Apolipoprotein analogue” or “apolipoprotein agonist” as used herein means a peptide, drug, or compound that mimics a function of native apolipoprotein either in vivo or in vitro. Native apolipoprotein include Apolipoprotein A-I (ApoA-I) (SEQ ID NO. 3), Apolipoprotein A-II (ApoA-II) (SEQ ID NO. 13), Apolipoprotein A-IV (ApoA-IV) (SEQ ID NO. 14), Apolipoprotein A-V (ApoA-V) (SEQ ID NO. 15), Apolipoprotein B (ApoB) (SEQ ID NO. 16), Apolipoprotein C-I (ApoC-I) (SEQ ID NO. 17), Apolipoprotein C-II (ApoC-II) (SEQ ID NO. 18), Apolipoprotein C-III (ApoC-III) (SEQ ID NO. 19), Apolipoprotein D (ApoD) (SEQ ID NO. 20), Apolipoprotein E (ApoE) (SEQ ID NO. 21), Apolipoprotein J (ApoJ) (SEQ ID NO. 22) and Apolipoprotein H (ApoH) (SEQ ID NO. 23). Apolipoprotein analogues may be incorporated, using methods known in the art, into a lipoprotein complex that functions as an HDL.

“Apolipoprotein peptide analogue” as used herein means a apolipoprotein analogue that is a peptide of between 10 and 200 amino acid residues in length, such peptides can contain either natural, or non-natural amino acids containing amide bonds. Apolipoprotein peptide analogues may be modified to improve their stability or bioavailability in vivo as known in the art and may contain organic compounds bound to the amino acid side chains through a variety of bonds.

“Apolipoprotein A-I analogue”, “Apo A-I analogue”, “apolipoprotein A-I agonist” or “Apo A-I agonist” as used herein mean a peptide that is derived from or mimics the function or structure of Apo A-I (SEQ ID NO. 3) either in vivo or in vitro and can be incorporated as part of a lipoprotein complex that functions as an HDL mimetic.

“Apolipoprotein complex”, apolipoprotein particle” “apolipoprotein”, “lipoprotein” or “lipoprotein complex” as used herein mean a composition comprising an apolipoprotein fraction and a lipid fraction and may be either man made, such as a synthetic HDL mimetic, or naturally occurring, such as circulating human HDL. Such compositions may be synthetic or isolated natural complexes as known in the art. Further, these compositions include both discoidal or micellar complexes or particles as known in the art. The apolipoprotein fraction comprises one or more proteins, peptides or peptide analogs including but not limited to apolipoprotein A-I analogues, native Human apolipoprotein A-I (SEQ ID NO. 3) or Human apolipoprotein A-I Milano variant (SEQ ID NO. 5) (i.e., ETC-216 analogue) and human Zaragoza variant Apolipoprotein A-I (SEQ ID NO. 12). The lipid fraction comprises both a surface coat and a hydrophobic core. The lipids comprise either the a surface coat (as in a discoidal particle) or a surface coat and a hydrophobic core (as in a spherical particle). The hydrophobic core is comprised of cholesterol, normally in the form of a cholesteryl ester, and triglycerides. At least ten apolipoproteins are known, including: ApoA-I (SEQ ID NO. 3), ApoA-II (SEQ ID NO. 13), ApoA-IV (SEQ ID NO. 14), ApoA-V (SEQ ID NO. 15), ApoB (SEQ ID NO. 16), ApoC-I (SEQ ID NO. 17), ApoC-II (SEQ ID NO. 18), ApoC-III (SEQ ID NO. 19), ApoD (SEQ ID NO. 20), ApoE (SEQ ID NO. 21), ApoJ (SEQ ID NO. 22) and ApoH (SEQ ID NO. 23). Other proteins such as LCAT (lecithin: cholesterol acyltransferase) (SEQ ID NO. 24), CETP (cholesteryl ester transfer protein) (SEQ ID NO. 25), PLTP (phospholipid transfer protein) (SEQ ID NO. 26 provides variant a, and additional isoforms include isoforms b, c, and d, as provided in Accession nos. NP_{—}872617.1, NP_{—}001229849.1, and NP_{—}001229850.1, respectively) and PON (paraoxonase) (SEQ ID NO. 27) are also found associated with lipoproteins as part of the lipoprotein complex. The surface coat of the lipid fraction comprises one or more phospholipids and may optionally comprise a combination of charged and neutral phospholipids as described in US patent application publication number 20060217312, herein incorporated by reference.

Lipoproteins for use in the present invention function in vitro and in vivo as an HDL mimetic. Charged phospholipid(s) can be positively or negatively charged at physiological pH. For example, the surface coat may contain charged lipids such as phosphatidylinositol, phosphatidylserine, phosphatidylglycerol phosphatidic acid in combination with neutral lipids such as phosphatidylcholine (lecithin) and sphingomyelin (SM) as known in the art (i.e., US patent application publication number 20060217312). The surface coat may also contain other types of lipids, such as triglycerides, cholesterol, cholesterol esters, lysophospholipids, and their various analogs and/or derivatives. The total amount of charged phospholipids(s) comprising the surface coat of the charged lipoprotein complexes can vary, but typically ranges from about 0.2 to 10 wt %. The total amount of neutral phospholipid(s) comprising the surface coat varies depending on the amount of charged phospholipid(s) and any optional lipids included. The surface coat will generally contain from about 90 to 99.8 wt % total neutral phospholipid(s). The neutral phospholipid can comprise a lecithin, a SM, or a mixture of lecithin, and SM. The lecithin and/or SM can comprise the bulk of the neutral phospholipid or, alternatively, the neutral phospholipid can include other neutral phospholipids in addition to the lecithin and/or SM. If the surface coat contains lecithin but not SM, the neutral phospholipid will typically comprise from about 5 to 100 wt % lecithin. If the surface coat contains a mixture of lecithin and SM, both the amount of the mixture comprising the total neutral phospholipid, and the relative amounts of the lecithin and SM comprising the mixture (i.e., lecithin:SM molar ratio) can vary. Typically, the neutral phospholipid will comprise from about 5 to 100 wt % of the lecithin/SM mixture. The molar ratio of lecithin to SM (lecithin:SM) can vary, but will typically range from about 20:1 to 1:20 or from 10:3 to 10:6 preferably from about 1:20 to 3:10. The lipid-to-apolipoprotein molar ratio of the lipoprotein complexes used in the present invention is from 2:1 to about 200:1 and preferably about 2:1 to 50:1. Lipoprotein complexes described herein can take on a variety of shapes, sizes and forms, including micellar structures; small, discoidal particles (akin to naturally-occurring pre-beta HDL particles; larger discoidal particles (akin to naturally-occurring alpha-HDL particles); and larger spherical particles that are akin to naturally-occurring HDL2 or HDL3. The desired size and shape of a lipoprotein complexes described can be controlled by adjusting the components and weight (or molar) ratios of the lipids comprising the lipid fraction, as well as the lipid:apolipoprotein molar ratio, as is know in the art (see, e.g., Barter et al., 1996, J. Biol. Chem. 271:4243-4250). For example, a discoidal particle or complex may contain a lipid fraction of about 90 to 99.8 wt % total neutral phospholipid(s) and about 0.2 to 10 wt % total negatively charged phospholipids(s). Such discoidal particles can be large (e.g., having an oblate diameter of about 10 to 14 nm) or small (e.g., having an oblate diameter of about 5 to 10 nm). The size of the discoidal particles can be controlled by adjusting the lipid:apolipoprotein molar ratio, as is known in the art (see, e.g., Barter et al., 1996, supra.). The sizes of the particles can be determined using, for example, size exclusion column chromatography.

“HDL mimetic” as used herein means a lipoprotein complex that mimics the function of native High density lipoprotein (HDL) either in vivo or in vitro. For example, an HDL mimetic may function in vivo to eliminate cholesterol or other lipids from extrahepatic tissues.

“About,” when immediately preceding a number or numeral means that the number or numeral ranges plus or minus 10%. For example, “about 1:1” ranges from 0.9:1 to 1.1:1.

“Alkyl” refers to a saturated branched, straight chain or cyclic hydrocarbon radical. Alkyl groups include saturated carbon chains which may be linear or branched or combinations thereof, unless the carbon chain is defined otherwise. Other groups having the prefix “alk”, such as alkoxy and alkanoyl, also may be linear or branched or combinations thereof, unless the carbon chain is defined otherwise. Typical alkyl groups include, but are not limited to, methyl, ethyl, propyl, isopropyl, butyl, isobutyl, sec or tert-butyl, pentyl, isopentyl, hexyl, heptyl, octyl, nonyl, and the like. In preferred embodiments, the alkyl groups are (C₁-C₆) alkyl.

“Alkenyl” refers to an unsaturated branched, straight chain or cyclic hydrocarbon radical having at least one carbon-carbon double bond. The radical may be in either the cis or trans conformation about the double bond(s). Typical alkenyl groups include, but are not limited to, allyl, ethenyl, propenyl, isopropenyl, butenyl, isobutenyl, tert-butenyl, pentenyl, hexenyl and the like. In preferred embodiments, the alkenyl group is (C₂-C₆) alkenyl.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” as used herein refers to an unsaturated cyclic hydrocarbon radical having a conjugated 7 electron system. Typical aryl groups include, but are not limited to, penta-2,4-diene, phenyl, naphthyl, anthracyl, azulenyl, chrysenyl, coronenyl, fluoranthenyl, indacenyl, idenyl, ovalenyl, perylenyl, phenalenyl, phenanthrenyl, picenyl, pleiadenyl, pyrenyl, pyranthrenyl, rubicenyl, and the like. In preferred embodiments, the aryl group is (C₁-C₂₀) aryl, with (C₅-C₁₀) being particularly preferred. The term “aryl” can also refer to an aryl group that is fused to a cycloalkyl or heterocycle. Preferred “aryls” are phenyl and naphthyl. Phenyl is generally the most preferred aryl group.

“Alkaryl” as used herein refers to a straight-chain alkyl, alkenyl or alkynyl group wherein one of the hydrogen atoms bonded to a terminal carbon is replaced with an aryl moiety. Typical alkaryl groups include, but are not limited to, benzyl, benzylidene, benzylidyne, benzenobenzyl, naphthenobenzyl and the like. In preferred embodiments, the alkaryl group is (C₆-C₂₆) alkaryl, i.e., the alkyl, alkenyl or alkynyl moiety of the alkaryl group is (C₁-C₆) or (C₂-C₆) and the aryl moiety is (C₅-C₂₀) or (C₄-C₂₀). In particularly preferred embodiments, the alkaryl group is (C₆-C₁₃) alkaryl, i.e., the alkyl, alkenyl or alkynyl moiety of the alkaryl group is (C₁-C₆) or (C₂-C₆) and the aryl moiety is (C₅-C₁₀) or (C₄-C₁₀).

“Heteroaryl” refers to an aryl moiety wherein one or more carbon atoms is replaced with another atom, such as N, P, O, S, As, Se, Si, Te, etc. Typical heteroaryl groups include, but are not limited to, acridarsine, acridine, arsanthridine, arsindole, arsindoline, carbazole, O-carboline, chromene, cinnoline, furan, imidazole, indazole, indole, indolizine, isoarsindole, isoarsinoline, isobenzofuran, isochromene, isoindole, isophosphoindole, isophosphinoline, isoquinoline, isothiazole, isoxazole, naphthyridine, perimidine, phenanthridine, phenanthroline, phenazine, phosphoindole, phosphinoline, phthalazine, pteridine, purine, pyran, pyrazine, pyrazole, pyridazine, pyridine, pyrimidine, pyrrole, pyrrolizine, quinazoline, quinoline, quinolizine, quinoxaline, selenophene, tellurophene, thiophene and xanthene. In preferred embodiments, the heteroaryl group is a 5-20 membered heteroaryl, with 5-10 membered aryl being particularly preferred.

“Alkheteroaryl” as used herein refers to a straight-chain alkyl, alkenyl or alkynyl group where one of the hydrogen atoms bonded to a terminal carbon atom is replaced with a heteroaryl moiety. In preferred embodiments, the alkheteroaryl group is 6-26 membered alkheteroaryl, i.e., the alkyl, alkenyl or alkynyl moiety of the alkheteroaryl is (C₁-C₆) or (C₂-C₆) and the heteroaryl is a 5-20-membered or 4-20-membered heteroaryl. In particularly preferred embodiments the alkheteroaryl is 6-13 membered alkheteroaryl, i.e., the alkyl, alkenyl or alkynyl moiety is (C₁-C₃) or (C₂-C₃) and the heteroaryl is a 5-10 membered heteroaryl.

“Substituted Alkyl, Alkynyl, Aryl, Alkaryl, Heteroaryl or Alkheteroaryl” as used herein refers to an alkyl, alkenyl, alkynyl, aryl, alkaryl, heteroaryl or alkheteroaryl group in which one or more hydrogen atoms is replaced with another substituent. Preferred substituents include —OR, —SR, —NRR, —NO₂—CN, halogen, —C(O)R, —C(O)OR and —C(O)NR, where each R is independently hydrogen, alkyl, alkenyl, alkynyl, aryl, alkaryl, heteroaryl or alkheteroaryl.

“Lower alkyl” or “C₁₋₇-alkyl”, alone or in combination with other groups, refers to a branched or straight-chain monovalent alkyl radical of one to seven carbon atoms. In preferred embodiments, the carbon has one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

“Lower alkoxy” or “C₁₋₇-alkoxy” refers to the group R′—O—, wherein R′ is lower alkyl. Examples of lower alkoxy groups are, for instance, methoxy, ethoxy, propoxy, isopropoxy, butoxy, isobutoxy and hexyloxy, with methoxy being especially preferred

“Lower alkoxyalkyl” or “C₁₋₇-alkoxy-C₁₋₇-alkyl” refers to a lower alkyl group as defined above which is mono- or multiply substituted with a lower alkoxy group as defined above. Examples of lower alkoxyalkyl groups are, for instance, —CH₂—O—CH₃, —CH₂—CH₂—O—CH₃, —CH₂—O—CH₂—CH₃ and the groups specifically exemplified herein. Most preferably, lower alkoxyalkyl is methoxyethyl.

“Lower alkylcarbonylamino” refers to the group —NH—CO—R″, wherein R″ is lower alkyl as defined above.

“Lower alkylaminocarbonyl” refers to the group —CO—NH—R″, wherein R″ is lower alkyl as defined above.

“Lower alkylsulfonyl” or “C₁₋₇-alkylsulfonyl” refers to the group R′—SO₂—, wherein R′ is lower alkyl. Examples of lower alkylsulfonyl groups include methanesulfonyl and ethanesulfonyl.

“Lower alkylsulfonylamino” or “C₁₋₇-alkylsulfonylamino” refers to the group R′—SO₂—NH—, wherein R′ is lower alkyl. A preferred lower alkylsulfonylamino group is methanesulfonylamino.

“Lower cycloalkylalkyl” or “C₃₋₇-cycloalkyl-C₁₋₇-alkyl” refers to a lower alkyl group as defined above which is mono- or multiply substituted with a cycloalkyl group as defined herein. Examples of lower cycloalkylalkyl groups are, for instance, —CH₂-cyclopropyl, —CH₂—CH₂-cyclopropyl, —CH₂-cyclopentyl and the groups specifically exemplified herein.

“Lower halogenalkyl” or “halogen-C₁₋₇-alkyl” refers to lower alkyl groups which are mono- or multiply substituted with halogen, preferably with fluoro or chloro, most preferably with fluoro. “Halogen-C₁₋₈-alkyl” refers to C₁₋₈ alkyl groups which are mono- or multiply substituted with halogen, preferably with fluoro or chloro, most preferably with fluoro. Examples of lower halogenalkyl groups are, for example, —CF₃, —CHF₂, —CH₂C₁, —CH₂CF₃, —CH(CF₃)₂, —CF₂—CF₃ and the groups specifically exemplified herein.

“Lower halogenalkoxy” or “halogen-C₁₋₇-alkoxy” refers to lower alkoxy groups as defined above wherein at least one of the hydrogen atoms of the lower alkoxy group is replaced by a halogen atom, preferably fluoro or chloro, most preferably fluoro. “Halogen-C₁₋₈-alkoxy” refers to C₁₋₈ alkoxy groups as defined above wherein at least one of the hydrogen atoms of the alkoxy group is replaced by a halogen atom. Among the preferred halogenated lower alkyl groups are trifluoromethoxy, difluoromethoxy, fluormethoxy and chloromethoxy, with trifluoromethoxy being especially preferred.

“Lower heteroarylalkyl” or “heteroaryl-C₁₋₈-alkyl” refers to lower alkyl groups as defined above wherein at least one of the hydrogen atoms of the lower alkyl group is replaced by a heteroaryl group as defined above.

“Lower hydroxyalkyl” or “hydroxy-C₁₋₇-alkyl” refers to lower alkyl groups as defined above wherein at least one of the hydrogen atoms of the lower alkyl group is replaced by a hydroxy group. Preferred are C₃₋₇-hydroxyalkyl groups. Examples of lower hydroxyalkyl groups are 2-hydroxybutyl, 3-hydroxy-2,2-dimethylpropyl and the groups specifically exemplified therein.

“Lower hydroxyalkoxyalkyl” or “hydroxy-C₁₋₇-alkoxy-C₁₋₇-alkyl” refers to lower alkoxyalkyl groups as defined above wherein at least one of the hydrogen atoms of the lower alkoxyalkyl group is replaced by a hydroxy group.

“Lower hydroxyhalogenalkyl” or “hydroxy-halogen-C₁₋₇-alkyl” refers to lower halogenalkyl groups as defined above herein which are additionally substituted with a hydroxy group. Examples of lower hydroxyhalogenalkyl groups are, for instance, 3,3,3-trifluoro-2-hydroxy-propyl and the groups specifically exemplified herein.

“Ac” as used herein refers to acetyl, which is CH₃C(═O)—.

“Alkylene” groups are alkyl groups that are difunctional rather than monofunctional. For example, methyl is an alkyl group and methylene (—CH₂—) is the corresponding alkylene group.

“Cycloalkyl” means a saturated carbocyclic ring having from 3 to 8 carbon atoms, unless otherwise stated (e.g., cycloalkyl may be defined as having one or more double bonds). The term also includes a cycloalkyl ring fused to an aryl group. Examples of cycloalkyl include cyclopropyl, cyclopentyl, cyclohexyl, cycloheptyl, and the like.

“Cycloalkenyl” means a non-aromatic carbocyclic ring having one or more double bonds.

“EDC” is 1-ethyl-3-(3-dimethylaminopropyl)carbodiimide.

“Heterocyclyl”, “heterocycle,” and “heterocyclic” means a fully or partially saturated or aromatic 5-6 membered ring containing 14 heteroatoms independently selected from N, S and O, unless otherwise stated.

“Benzoheterocycle” represents a phenyl ring fused to a 5-6-membered heterocyclic ring having 1-2 heteroatoms, each of which is O, N, or S, where the heterocyclic ring may be saturated or unsaturated. Examples include indole, benzofuran, 2,3-dihydrobenzofuran and quinoline.

As used herein when referring to an ApoA-I analogue peptide, the number of terminal —NH₂ groups is zero where R¹ is an amino protecting group and is 1 where R¹ is H.

As used herein when referring to an ApoA-I analogue peptide, the number of terminal —COOH groups is zero where R² is a carboxyl protecting group and is 1 where R² is OH.

“DIPEA” is diisopropylethylamine.

“Halogen” includes fluorine, chlorine, bromine and iodine.

“HOBT” is 1-Hydroxybenzotriazole.

“IPAC” is isopropyl acetate.

“Me” represents methyl.

The substituent “tetrazole” means a 2H-tetrazol-5-yl substituent group and tautomers thereof. Optical Isomers-Diastereomers-Geometric Isomers-Tautomers.

The term “composition” or “pharmaceutical composition” is intended to encompass a product comprising the active ingredient(s), and the inert ingredient(s) that make up the carrier, as well as any product which results, directly or indirectly, from combination, complexed or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound or apolipoprotein complex for use in the present invention and a pharmaceutically acceptable carrier

A “mammal,” as used herein unless otherwise defined, refers to a human, mouse, rat, guinea pig, dog, cat, horse, cow, pig, or non-human primate, such as a monkey, chimpanzee, or baboon. In one embodiment, the mammal is a human.

An “effective amount,” when used in connection with an apolipoprotein complex or small molecule compound, for use in the present invention, is an amount that is effective for treating LVDD.

The terms “to treat”, “treatment”, “treating” and the like as used herein in reference to the present invention mean to improve, ameliorate, prevent or cure left ventricular diastolic dysfunction in a human having left ventricular diastolic dysfunction.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound or peptide is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

“Amino acid residue,” “amino acid,” or “residue” as used herein unless otherwise defined, includes genetically encoded amino acid residues and non-genetically encoded amino acid residues.

As used herein, the abbreviations for the genetically encoded L-enantiomeric amino acids are conventional and are as follows:

| | | 3 letter |
|---|---|---|
| Amino Acid | 1 letter abbreviation | abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

The abbreviations used for the D-enantiomers of the genetically encoded amino acids are lower-case equivalents of the one-letter symbols. For example, “P” designates L-proline and “p” designates D-proline.

Non-genetically encoded amino acid residues or non-natural amino acids include, but are not limited to, β-alanine (β-Ala); 2,3-diaminopropionic acid (Dpr); nipecotic acid (Nip); pipecolic acid (Pip); ornithine (Orn); citrulline (Cit); t-butylalanine (t-BuA); 2-t-butylglycine (t-BuG); N-methylisoleucine (MeIle); phenylglycine (PhG); cyclohexylalanine (ChA); norleucine (Nle); naphthylalanine (Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe (pNH₂)); N-methyl valine (MeVal); homocysteine (hCys), homophenylalanine (hPhe); homoserine (hSer); hydroxyproline (Hyp); homoproline (hPro); and the corresponding D-enantiomer of each of the foregoing, e.g., D-β-Ala, D-Dpr, D-Nip, D-Orn, D-Cit, D-t-BuA, D-t-BuG, D-MeIle, D-PhG, D-ChA, D-Nle, D-Nal, D-Phe(4-Cl), D-Phe(2-F), D-Phe(3-F), D-Phe(4-F), D-Pen, D-Tic, D-Thi, D-MSO, D-hArg, D-AcLys, D-Dbu, D-Dab, D-Phe(pNH₂), D-MeVal, D-hCys, D-hPhe, D-hSer, D-Hyp, and D-hPro. Other non-genetically encoded amino acid residues include 3-aminopropionic acid; 4-aminobutyric acid; isonipecotic acid (Inp); aza-pipecolic acid (azPip); aza-proline (azPro); α-aminoisobutyric acid (Aib); ε-aminohexanoic acid (Aha); δ-aminovaleric acid (Ava); N-methylglycine (MeGly).

“Chiral,” as used herein to refer to an amino acid residue, means an amino acid residue having at least one chiral center. In one embodiment, the chiral amino acid residue is an L-amino acid residue. Examples of L-amino acid residues include, but are not limited to, Ala, Arg, Asn, Asp, Cys, Gln, Glu, His, Ile, Leu, Lys, Met, Phe, Pro, Ser, Thr, Trp, Tyr, Val, β-Ala, Dpr, Nip, Orn, Cit, t-BuA, t-BuG, MeIle, PhG, ChA, Nle, Nal, Phe(4-Cl), Phe(2-F), Phe(3-F), Phe(4-F), Pen, Tic, Thi, MSO, hArg, AcLys, Dbu, Dab, Phe(pNH₂), MeVal, hCys, hPhe, hSer, Hyp, and hPro. In one embodiment, the chiral amino acid residue is a D-amino acid residue. Examples of D-amino acid residues include, but are not limited to D-Ala, D-Arg, D-Asn, D-Asp, D-Cys, D-Gln, D-Glu, D-His, D-Ile, D-Leu, D-Lys, D-Met, D-Phe, D-Pro, D-Ser, D-Thr, D-Trp, D-Tyr, D-Val, D-Dpr, D-Nip, D-Pip, D-Orn, D-Cit, D-t-BuA, D-t-BuG, D-MeIle, D-PhG, D-ChA, D-Nle, D-Nal, D-Phe(4-Cl), D-Phe(2-F), D-Phe(3-F), D-Phe(4-F), D-Pen, D-Tic, D-Thi, D-MSO, D-hArg, D-AcLys, D-Dbu, D-Dab, D-Phe (pNH₂), D-MeVal, D-hCys, D-hPhe, D-hSer, D-Hyp, and D-hPro.

“Achiral,” as used herein to refer to an amino acid residue, means an amino acid residue that does not have a chiral center. Examples of achiral amino acid residues include, but are not limited to, Gly, Inp, Aib, Aha, Ava, MeGly, azPip, and azPro.

“Aliphatic amino acid residue,” as used herein unless otherwise defined, refers to an amino acid residue having an aliphatic hydrocarbon side chain. Aliphatic amino acid residues include, but are not limited to, Ala (A), Val (V), Leu (L), Ile (I), Pro (P), azPro, Pip, azPip, β-Ala, Aib, t-BuA, t-BuG, MeIle, ChA, Nle, MeVal, Inp, Nip, hPro, D-Ala, D-Val, D-Leu, D-Ile, D-Pro, D-β-BuA, D-t-BuG, D-MeIle, D-Nle, D-MeVal, D-Nip, D-Pip, D-ChA, and D-hPro. In one embodiment, the aliphatic amino acid residue is an L-amino acid residue. In another embodiment, the aliphatic amino acid residue is a D-amino acid residue. In another embodiment, the aliphatic amino acid residue is an achiral amino acid residue.

“Hydrophilic amino acid residue,” as used herein unless otherwise defined, refers to an amino acid residue exhibiting a hydrophobicity of less than zero according to the normalized consensus hydrophobicity scale of Eisenberg et al., 1984, J. Mol. Biol. 179:125-I42. Hydrophilic amino acid residues include, but are not limited to, Pro (P), Gly (G), Thr (T), Ser (S), His (H), Glu (E), Asn (N), Gln (Q), Asp (D), Lys (K) Arg (R), Dpr, Orn, Cit, Pen, MSO, hArg, AcLys, Dbu, Dab, Phe(p-NH₂), hCys, hSer, Hyp, D-Pro, D-Thr, D-Ser, D-His, D-Glu, D-Asn, D-Gln, D-Asp, D-Lys, D-Arg, D-Dpr, D-Orn, D-Cit, D-Pen, D-MSO, D-hArg, D-AcLys, D-Dbu, D-Dab, D-Phe(p-NH₂), D-hCys, D-hSer, and D-Hyp. Other hydrophilic amino acid residues include, but are not limited to, C₁₋₄ lateral chain analogs having the following formulas:

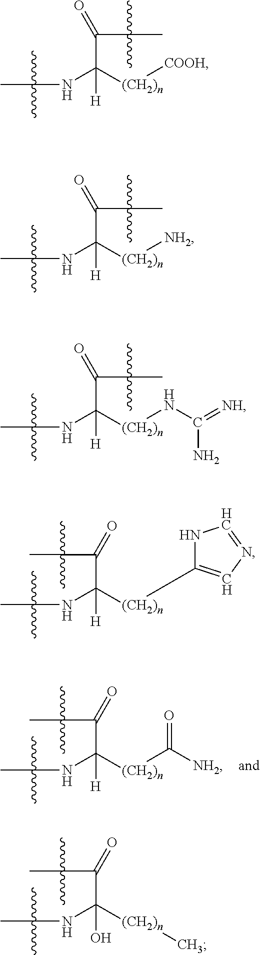

wherein n is an integer from 1 to 4. In one embodiment, the hydrophilic amino acid residue is an L-amino acid residue. In another embodiment, the hydrophilic amino acid residue is a D-amino acid residue. In another embodiment, the hydrophilic amino acid residue is an achiral amino acid residue. In another embodiment, the hydrophilic amino acid residue is an acidic L-amino acid residue, an acidic D-amino acid residue, or an acidic achiral amino acid residue. In another embodiment, the hydrophilic amino acid residue is a basic L-amino acid residue, a basic D-amino acid residue, or a basic achiral amino acid residue.

“Hydrophobic amino acid residue,” as used herein unless otherwise defined, refers to an amino acid residue exhibiting a hydrophobicity of greater than zero according to the normalized consensus hydrophobicity scale of Eisenberg, 1984, J Mol. Biol. 179:125-I42. Hydrophobic amino acid residues include, but are not limited to, Ile (I), Phe (F), Val (V), Leu (L), Trp (W), Met (M), Ala (A), Gly (G), Tyr (Y), β-Ala, Nip, t-BuA, t-BuG, MeIle, PhG, ChA, Nle, Nal, Phe(4-Cl), Phe(2-F), Phe(3-F), Phe(4-F), Tic, Thi, MeVal, hPhe, hPro, 3-aminopropionic acid, 4 aminobutryic acid, Inp, Aib, Aha, Ava, MeGly, D-Pro, D-Ile, D-Phe, D-Val, D-Leu, D-Trp, D-Met, D-Ala, D-Tyr, D-β-Ala, D-Nip, D-t-BuA, D-t-BuG, D-MeIle, D-PhG, D-ChA, D-Nle, D-Nal, D-Phe(4-Cl), D-Phe(2-F), D-Phe(3-F), D-Phe(4-F), D-Tic, D-Thi, D-MeVal, D-hPhe, and D-hPro. Other hydrophobic amino acids include, but are not limited to, C₁₋₄ lateral chain analogs having the following formulas:

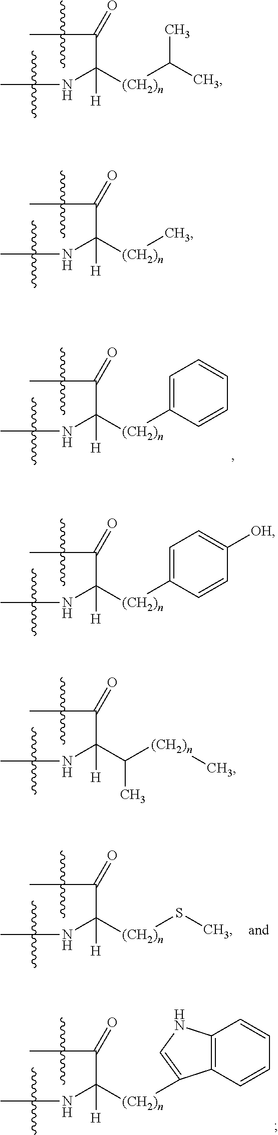

wherein n is an integer from 1 to 4. In one embodiment, the hydrophobic amino acid residue is an L-amino acid residue. In another embodiment, the hydrophobic amino acid residue is a D-amino acid residue. In another embodiment, the hydrophobic amino acid residue is an achiral amino acid residue.

“Polar amino acid residue,” as used herein unless otherwise defined, refers to a hydrophilic amino acid residue having a side chain that is uncharged at physiological pH, but which has at least one bond in which the pair of electrons shared in common by two atoms is held more closely by one of the atoms. Polar amino acid residues include, but are not limited to, Asn (N), Gln (Q), Ser (S), Thr (T), Cit, Pen, MSO, AcLys, hCys, hSer, Hyp, D-Asn, D-Gln, D-Ser, D-Thr, D-Cit, D-Pen, D-MSO, D-AcLys, D-hCys, D-hSer, and D-Hyp. Other polar amino acids include, but are not limited to, C₁₋₄ lateral chain analogs having the following formulas:

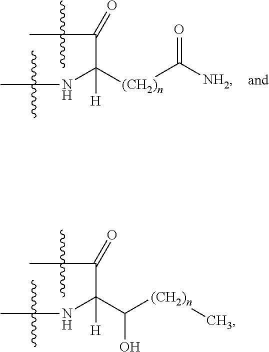

wherein n is an integer from 1 to 4. In one embodiment, the polar amino acid residue is an L-amino acid residue. In another embodiment, the polar amino acid residue is a D-amino acid residue. In another embodiment, the polar amino acid residue is an achiral amino acid residue.

“Acidic amino acid residue,” as used herein unless otherwise defined, refers to a hydrophilic amino acid residue having a side chain pK value of less than 7. Acidic amino acid residues typically have negatively charged side chains at physiological pH due to loss of a hydrogen ion. Acidic amino acid residues include, but are not limited to, Glu (E), Asp (D), D-Glu, and D-Asp. Other acidic amino acids include, but are not limited to, C₁₋₄ lateral chain analogs having the following formula:

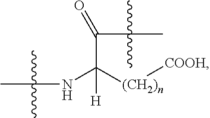

wherein n is an integer from 1 to 4. In one embodiment, the acidic amino acid residue is an L-amino acid residue. In another embodiment, the acidic amino acid residue is a D-amino acid residue. In another embodiment, the acidic amino acid residue is an achiral amino acid residue.

“Basic amino acid residue,” as used herein unless otherwise defined, refers to a hydrophilic amino acid residue having a side chain pK value of greater than 7. Basic amino acid residues typically have positively charged side chains at physiological pH due to association with a hydronium ion. Basic amino acid residues include, but are not limited to, His (H), Arg (R), Lys (K), Dpr, Orn, hArg, Dbu, Dab, Phe(p-NH₂), D-His, D-Arg, D-Lys, D-Dpr, D-Orn, D-hArg, D-Dbu, D-Dab, and D-Phe(p-NH₂). Other basic amino acid residues include, but are not limited to, C₁₋₄ lateral chain analogs having the following formulas:

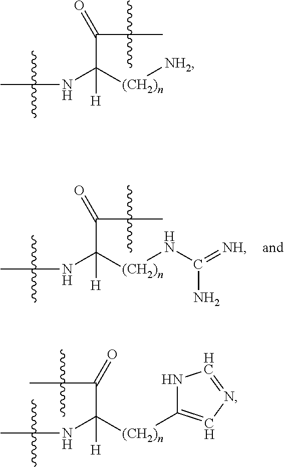

wherein n is an integer from 1 to 4. In one embodiment, the basic amino acid residue is an C-amino acid residue. In another embodiment, the basic amino acid residue is a D-amino acid residue. In another embodiment, the basic amino acid residue is an achiral amino acid residue.

“Nonpolar amino acid residue,” as used herein unless otherwise defined, refers to a hydrophobic amino acid residue having a side chain that is uncharged at physiological pH and which has bonds in which the pair of electrons shared in common by two atoms is held substantially equally by each of the two atoms (i.e., the side chain is not polar). Non-polar amino acid residues include, but are not limited to, Leu (L), Val (V), Ile (I), Met (M), Gly (G), Ala (A), Pro (P), azPro, Pip, azPip, β-Ala, Nip, t-BuG, MeIle, ChA, Nle, MeVal, hPro, 3-aminopropionic acid, 4-aminobutyric acid, Inp, Aib, Aha, Ava, MeGly, D-Leu, D-Val, D-Ile, D-Met, D-Ala, D-Pro, D-β-Ala, D-Inp, D-t-BuG, D-MeIle, D-ChA, D-Nle, D-MeVal, D-Nip, D-Pip, and D-hPro. Other non-polar amino acid residues include, but are not limited to, C₁₋₄ lateral chain analogs having the following formulas:

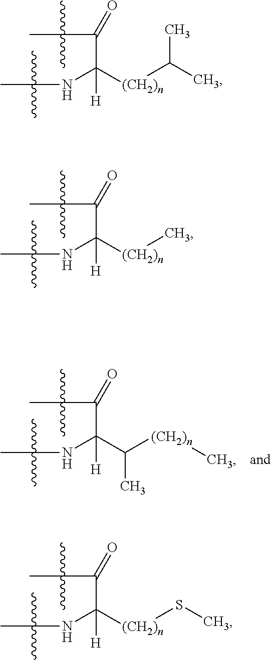

wherein n is an integer from 1 to 4. In one embodiment, the non-polar amino acid residue is an L-amino acid residue. In another embodiment, the non-polar amino acid residue is a D-amino acid residue. In another embodiment, the non-polar amino acid residue is an achiral amino acid residue.

“Aromatic amino acid residue,” as used herein unless otherwise defined, refers to a hydrophobic amino acid residue with a side chain having at least one aromatic or heteroaromatic ring. The aromatic or heteroaromatic ring can contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR where each R is independently (C₁-C₆) alkyl, substituted (C₁-C₆) alkyl, 5-26-membered aryl, and substituted 5-26-membered aryl. Aromatic amino acid residues include, but are not limited to, Phe (F), Tyr (Y), Trp (W), PhG, Nal, Phe(4-Cl), Phe(2-F), Phe(3-F), Phe(4-F), Tic, Thi, hPhe, D-Phe, D-Tyr and D-Trp, D-PhG, D-Nal, D-Phe(4-Cl), D-Phe(2-F), D-Phe(3-F), D-Phe(4-F), D-Tic, D-Thi, and D-hPhe. Other aromatic amino acid residues include, but are not limited to, C₁₋₄ lateral chain analogs having the following formulas:

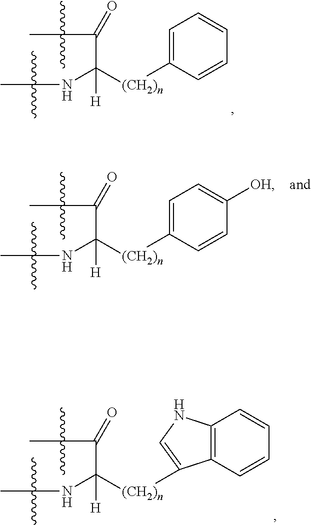

wherein n is an integer from 1 to 4. In one embodiment, the aromatic amino acid residue is an L-amino acid residue. In another embodiment, the aromatic amino acid residue is a D-amino acid residue. In another embodiment, the aromatic amino acid residue is an achiral amino acid residue.

### II. Apolipoprotein Complexes for the Treatment of Left Ventricular Diastolic Dysfunction (LVDD)

The present invention relates to pharmaceutical compositions for the treatment of left ventricular diastolic dysfunction. In one embodiment the invention provides pharmaceutical compositions comprising an apolipoprotein complex for treatment of LVDD.

Apolipoprotein complexes for use in the present invention include those described in US application publication number US2006/0217312, which discloses lipoprotein complexes having a protein fraction comprising Human preproApoA-I (SEQ ID NO. 1), (SEQ. ID. NO. 1), Human proApoA-I (SEQ ID NO. 2), (SEQ. ID. NO. 2), Human ApoA-I (SEQ ID NO. 3) (SEQ. ID. NO. 3), ApoA-I Milano (SEQ ID NO. 11), ApoA-I Paris variant (SEQ. ID. NO. 10) or a apoA-I analogue. Exemplary human ApoA-I (SEQ ID NO. 3) protein sequences and apolipoprotein complexes include but are not limited to those listed below:

SEQ ID NO. 1: preproApo A-I
MKAAVLTLAVLFLTGSQARHFWQQDEPPQSPWDRVKDLATVYVDVLKDSGRDYVSQFEGSALGKQLNLKL
LDNWDSVTSTFSKLREQLGPVTQEFWDNLEKETEGLRQEMSKDLEEVKAKVQPYLDDFQKKWQEEMELYR
QKVEPLRAELQEGARQKLHELQEKLSPLGEEMRDRARAHVDALRTHLAPYSDELRQRLAARLEALKENGG
ARLAEYHAKATEHLSTLSEKAKPALEDLRQGLLPVLESFKVSFLSALEEYTKKLNTQ
SEQ ID NO. 2: proApo A-I (cleaved signal peptide MKAAVLTLAVLFLTGSQARHFWQQ from preproapo A-I)
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNTQ
SEQ ID NO. 3: mature human Apo A-I (cleaved terminal Q from proapo A-I)
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 4: human Milano variant of preproApoA-I
MKAAVLTLAVLFLTGSQARHFWQQDEPPQSPWDRVKDLATVYVDVLKDSGRDYVSQFEGSALGKQLNLKLLDNWDS
VTSTFSKLREQLGPVTQEFWDNLEKETEGLRQEMSKDLEEVKAKVQPYLDDFQKKWQEEMELYRQKVEPLRAELQEG
ARQKLHELQEKLSPLGEEMRDRARAHVDALRTHLAPYSDELRQCLAARLEALKENGGARLAEYHAKATEHLSTLSEKAK
PALEDLRQGLLPVLESFKVSFLSALEEYTKKLNTQ
SEQ ID NO. 5: human Milano variant of proApoA-I
DEPPQSPWDRVKDLATVYVDVLKDSGRDYVSQFEGSALGKQLNLKLLDNWDSVTSTFSKLREQLGPVTQEFWDNLEK
ETEGLRQEMSKDLEEVKAKVQPYLDDFQKKWQEEMELYRQKVEPLRAELQEGARQKLHELQEKLSPLGEEMRDRARA
HVDALRTHLAPYSDELRQCLAARLEALKENGGARLAEYHAKATEHLSTLSEKAKPALEDLRQGLLPVLESFKVSFLSALEEY
TKKLNTQ
SEQ ID NO. 6: human Paris variant of preproApoA-I
MKAAVLTLAVLFLTGSQARHFWQQDEPPQSPWDRVKDLATVYVDVLKDSGRDYVSQFEGSALGKQLNLKLLDNWDS
VTSTFSKLREQLGPVTQEFWDNLEKETEGLRQEMSKDLEEVKAKVQPYLDDFQKKWQEEMELYRQKVEPLRAELQEG
ARQKLHELQEKLSPLGEEMRDCARAHVDALRTHLAPYSDELRQRLAARLEALKENGGARLAEYHAKATEHLSTLSEKAK
PALEDLRQGLLPVLESFKVSFLSALEEYTKKLNTQ
SEQ ID NO. 7: human Paris variant of proApoA-I
DEPPQSPWDRVKDLATVYVDVLKDSGRDYVSQFEGSALGKQLNLKLLDNWDSVTSTFSKLREQLGPVTQEFWDNLEK
ETEGLRQEMSKDLEEVKAKVQPYLDDFQKKWQEEMELYRQKVEPLRAELQEGARQKLHELQEKLSPLGEEMRDCARA
HVDALRTHLAPYSDELRQRLAARLEALKENGGARLAEYHAKATEHLSTLSEKAKPALEDLRQGLLPVLESFKVSFLSALEEY
TKKLNTQ
SEQ ID NO. 8: human Zaragoza variant of preproApoA-I
MKAAVLTLAVLFLTGSQARHFWQQDEPPQSPWDRVKDLATVYVDVLKDSGRDYVSQFEGSALGKQLNLKLLDNWDS
VTSTFSKLREQLGPVTQEFWDNLEKETEGLRQEMSKDLEEVKAKVQPYLDDFQKKWQEEMELYRQKVEPLRAELQEG
ARQKLHELQEKLSPRGEEMRDRARAHVDALRTHLAPYSDELRQRLAARLEALKENGGARLAEYHAKATEHLSTLSEKAK
PALEDLRQGLLPVLESFKVSFLSALEEYTKKLNTQ
SEQ ID NO. 9: human Zaragoza variant of proApoA-I
DEPPQSPWDRVKDLATVYVDVLKDSGRDYVSQFEGSALGKQLNLKLLDNWDSVTSTFSKLREQLGPVTQEFWDNLEK
ETEGLRQEMSKDLEEVKAKVQPYLDDFQKKWQEEMELYRQKVEPLRAELQEGARQKLHELQEKLSPRGEEMRDRARA
HVDALRTHLAPYSDELRQRLAARLEALKENGGARLAEYHAKATEHLSTLSEKAKPALEDLRQGLLPVLESFKVSFLSALEEY
TKKLNTQ
SEQ ID NO. 10: Natural variant 151 R to C in Paris
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDCARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 11: Natural variant 173 1 R to C in Milano; associated with decreased HDL levels and
moderate increases in triglycerides; no evidence of association with premature vascular disease.
[dbSNP:rs28931573] Ref. 39 VAR_000624
DEPPQSPWDRVKDLATVYVDVLKDSGRDYVSQFEGSALGKQLNLKLLDNWDSVTSTFSKLREQLGPVTQEFWDNLEK
ETEGLRQEMSKDLEEVKAKVQPYLDDFQKKWQEEMELYRQKVEPLRAELQEGARQKLHELQEKLSPLGEEMRDRARA
HVDALRTHLAPYSDELRQCLAARLEALKENGGARLAEYHAKATEHLSTLSEKAKPALEDLRQGLLPVLESFKVSFLSALEEY
TKKLNT
SEQ ID NO. 12: Natural variant 144 L to R in Zaragoza
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPRGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 13: human Apolipoprotein A-II (ApoA-II, which is residues 24-99 in the sequence below)
>sp|P02652|APOA2_HUMAN Apolipoprotein A-II OS = Homo sapiens GN = APOA2 PE = 1 SV = 1
MKLLAATVLLLTICSLEGALVRRQAKEPCVESLVSQYFQTVTDYGKDLMEKVKSPELQAE
AKSYFEKSKEQLTPLIKKAGTELVNFLSYFVELGTQPATQ
SEQ ID NO. 14: human Apolipoprotein A-IV (ApoA-IV, which is residues 21-396 in the sequence below)
>sp|P06727|APOA4_HUMAN Apolipoprotein A-IV OS = Homo sapiens GN = APOA4 PE = 1 SV = 3
MFLKAVVLTLALVAVAGARAEVSADQVATVMWDYFSQLSNNAKEAVEHLQKSELTQQLNALFQDKLGEVNTYAGDL
QKKLVPFATELHERLAKDSEKLKEEIGKELEELRARLLPHANEVSQKIGDNLRELQQRLEPYADQLRTQVNTQAEQLRRQ
LTPYAQRMERVLRENADSLQASLRPHADELKAKIDQNVEELKGRLTPYADEFKVKIDQTVEELRRSLAPYAQDTQEKLN
HQLEGLTFQMKKNAEELKARISASAEELRQRLAPLAEDVRGNLRGNTEGLQKSLAELGGHLDQQVEEFRRRVEPYGENF
NKALVQQMEQLRQKLGPHAGDVEGHLSFLEKDLRDKVNSFFSTFKEKESQDKTLSLPELEQQQEQQQEQQQEQVQM
LAPLES
SEQ ID NO. 15: human Apolipoprotein A-V (ApoA-V, which is residues 24-366 in the sequence below)
>sp|Q6Q788|APOA5_HUMAN Apolipoprotein A-V OS = Homo sapiens GN = APOA5 PE = 1 SV = 1
MASMAAVLTWALALLSAFSATQARKGFWDYFSQTSGDKGRVEQIHQQKMAREPATLKDSLEQDLNNMNKFLEKLRP
LSGSEAPRLPQDPVGMRRQLQEELEEVKARLQPYMAEAHELVGWNLEGLRQQLKPYTMDLMEQVALRVQELQEQLR
VVGEDTKAQLLGGVDEAWALLQGLQSRVVHHTGRFKELFHPYAESLVSGIGRHVQELHRSVAPHAPASPARLSRCVQV
LSRKLTLKAKALHARIQQNLDQLREELSRAFAGTGTEEGAGPDPQMLSEEVRQRLQAFRQDTYLQIAAFTRAIDQETEEV
QQQLAPPPPGHSAFAPEFQQTDSGKVLSKLQARLDDLWEDITHSLHDQGHSHLGDP
SEQ ID NO. 16: human Apolipoprotein B (ApoB, where ApoB-100 is residues 28-4563 and ApoB-48 is
residues 28-2179 in the sequence below)
>sp|P04114|APOB_HUMAN Apolipoprotein B-100 OS = Homo sapiens GN = APOB PE = 1 SV = 2
MDPPRPALLALLALPALLLLLLAGARAEEEMLENVSLVCPKDATRFKHLRKYTYNYEAESSSGVPGTADSRSATRINCKVE
LEVPQLCSFILKTSQCTLKEVYGFNPEGKALLKKTKNSEEFAAAMSRYELKLAIPEGKQVFLYPEKDEPTYILNIKRGIISALL
VPPETEEAKQVLFLDTVYGNCSTHFTVKTRKGNVATEISTERDLGQCDRFKPIRTGISPLALIKGMTRPLSTLISSSQSCQY
TLDAKRKHVAEAICKEQHLFLPFSYKNKYGMVAQVTQTLKLEDTPKINSRFFGEGTKKMGLAFESTKSTSPPKQAEAVLK
TLQELKKLTISEQNIQRANLFNKLVTELRGLSDEAVTSLLPQLIEVSSPITLQALVQCGQPQCSTHILQWLKRVHANPLLID
VVTYLVALIPEPSAQQLREIFNMARDQRSRATLYALSHAVNNYHKTNPTGTQELLDIANYLMEQIQDDCTGDEDYTYLIL
RVIGNMGQTMEQLTPELKSSILKCVQSTKPSLMIQKAAIQALRKMEPKDKDQEVLLQTFLDDASPGDKRLAAYLMLMR
SPSQADINKIVQILPWEQNEQVKNFVASHIANILNSEELDIQDLKKLVKEALKESQLPTVMDFRKFSRNYQLYKSVSLPSL
DPASAKIEGNLIFDPNNYLPKESMLKTTLTAFGFASADLIEIGLEGKGFEPTLEALFGKQGFFPDSVNKALYWVNGQVPD
GVSKVLVDHFGYTKDDKHEQDMVNGIMLSVEKLIKDLKSKEVPEARAYLRILGEELGFASLHDLQLLGKLLLMGARTLQ
GIPQMIGEVIRKGSKNDFFLHYIFMENAFELPTGAGLQLQISSSGVIAPGAKAGVKLEVANMQAELVAKPSVSVEFVTN
MGIIIPDFARSGVQMNTNFFHESGLEAHVALKAGKLKFIIPSPKRPVKLLSGGNTLHLVSTTKTEVIPPLIENRQSWSVCK
QVFPGLNYCTSGAYSNASSTDSASYYPLTGDTRLELELRPTGEIEQYSVSATYELQREDRALVDTLKFVTQAEGAKQTEAT
MTFKYNRQSMTLSSEVQIPDFDVDLGTILRVNDESTEGKTSYRLTLDIQNKKITEVALMGHLSCDTKEERKIKGVISIPRLQ
AEARSEILAHWSPAKLLLQMDSSATAYGSTVSKRVAWHYDEEKIEFEWNTGTNVDTKKMTSNFPVDLSDYPKSLHMY
ANRLLDHRVPQTDMTFRHVGSKLIVAMSSWLQKASGSLPYTQTLQDHLNSLKEFNLQNMGLPDFHIPENLFLKSDGRV
KYTLNKNSLKIEIPLPFGGKSSRDLKMLETVRTPALHFKSVGFHLPSREFQVPTFTIPKLYQLQVPLLGVLDLSTNVYSNLYN
WSASYSGGNTSTDHFSLRARYHMKADSVVDLLSYNVQGSGETTYDHKNTFTLSYDGSLRHKFLDSNIKFSHVEKLGNNP
VSKGLLIFDASSSWGPQMSASVHLDSKKKQHLFVKEVKIDGQFRVSSFYAKGTYGLSCQRDPNTGRLNGESNLRFNSSY
LQGTNQITGRYEDGTLSLTSTSDLQSGIIKNTASLKYENYELTLKSDTNGKYKNFATSNKMDMTFSKQNALLRSEYQADY
ESLRFFSLLSGSLNSHGLELNADILGTDKINSGAHKATLRIGQDGISTSATTNLKCSLLVLENELNAELGLSGASMKLTTNG
RFREHNAKFSLDGKAALTELSLGSAYQAMILGVDSKNIFNFKVSQEGLKLSNDMMGSYAEMKFDHTNSLNIAGLSLDFS
SKLDNIYSSDKFYKQTVNLQLQPYSLVTTLNSDLKYNALDLTNNGKLRLEPLKLHVAGNLKGAYQNNEIKHIYAISSAALSA
SYKADTVAKVQGVEFSHRLNTDIAGLASAIDMSTNYNSDSLHFSNVFRSVMAPFTMTIDAHTNGNGKLALWGEHTGQ
LYSKFLLKAEPLAFTFSHDYKGSTSHHLVSRKSISAALEHKVSALLTPAEQTGTWKLKTQFNNNEYSQDLDAYNTKDKIGV
ELTGRTLADLTLLDSPIKVPLLLSEPINIIDALEMRDAVEKPQEFTIVAFVKYDKNQDVHSINLPFFETLQEYFERNRQTIIVV
LENVQRNLKHINIDQFVRKYRAALGKLPQQANDYLNSFNWERQVSHAKEKLTALTKKYRITENDIQIALDDAKINFNEKL
SQLQTYMIQFDQYIKDSYDLHDLKIAIANIIDEIIEKLKSLDEHYHIRVNLVKTIHDLHLFIENIDFNKSGSSTASWIQNVDTK
YQIRIQIQEKLQQLKRHIQNIDIQHLAGKLKQHIEAIDVRVLLDQLGTTISFERINDILEHVKHFVINLIGDFEVAEKINAFRA
KVHELIERYEVDQQIQVLMDKLVELAHQYKLKETIQKLSNVLQQVKIKDYFEKLVGFIDDAVKKLNELSFKTFIEDVNKFLD
MLIKKLKSFDYHQFVDETNDKIREVTQRLNGEIQALELPQKAEALKLFLEETKATVAVYLESLQDTKITLIINWLQEALSSAS
LAHMKAKFRETLEDTRDRMYQMDIQQELQRYLSLVGQVYSTLVTYISDWWTLAAKNLTDFAEQYSIQDWAKRMKAL
VEQGFTVPEIKTILGTMPAFEVSLQALQKATFQTPDFIVPLTDLRIPSVQINFKDLKNIKIPSRFSTPEFTILNTFHIPSFTIDF
VEMKVKIIRTIDQMLNSELQWPVPDIYLRDLKVEDIPLARITLPDFRLPEIAIPEFIIPTLNLNDFQVPDLHIPEFQLPHISHTI
EVPTFGKLYSILKIQSPLFTLDANADIGNGTTSANEAGIAASITAKGESKLEVLNFDFQANAQLSNPKINPLALKESVKFSSK
YLRTEHGSEMLFFGNAIEGKSNTVASLHTEKNTLELSNGVIVKINNQLTLDSNTKYFHKLNIPKLDFSSQADLRNEIKTLLK
AGHIAWTSSGKGSWKWACPRFSDEGTHESQISFTIEGPLTSFGLSNKINSKHLRVNQNLVYESGSLNFSKLEIQSQVDSQ
HVGHSVLTAKGMALFGEGKAEFTGRHDAHLNGKVIGTLKNSLFFSAQPFEITASTNNEGNLKVRFPLRLTGKIDFLNNYA
LFLSPSAQQASWQVSARFNQYKYNQNFSAGNNENIMEAHVGINGEANLDFLNIPLTIPEMRLPYTIITTPPLKDFSLWEK
TGLKEFLKTTKQSFDLSVKAQYKKNKHRHSITNPLAVLCEFISQSIKSFDRHFEKNRNNALDFVTKSYNETKIKFDKYKAEK
SHDELPRTFQIPGYTVPVVNVEVSPFTIEMSAFGYVFPKAVSMPSFSILGSDVRVPSYTLILPSLELPVLHVPRNLKLSLPDF
KELCTISHIFIPAMGNITYDFSFKSSVITLNTNAELFNQSDIVAHLLSSSSSVIDALQYKLEGTTRLTRKRGLKLATALSLSNKF
VEGSHNSTVSLTTKNMEVSVATTTKAQIPILRMNFKQELNGNTKSKPTVSSSMEFKYDFNSSMLYSTAKGAVDHKLSLE
SLTSYFSIESSTKGDVKGSVLSREYSGTIASEANTYLNSKSTRSSVKLQGTSKIDDIWNLEVKENFAGEATLQRIYSLWEHST
KNHLQLEGLFFTNGEHTSKATLELSPWQMSALVQVHASQPSSFHDFPDLGQEVALNANTKNQKIRWKNEVRIHSGSF
QSQVELSNDQEKAHLDIAGSLEGHLRFLKNIILPVYDKSLWDFLKLDVTTSIGRRQHLRVSTAFVYTKNPNGYSFSIPVKVL
ADKFIIPGLKLNDLNSVLVMPTFHVPFTDLQVPSCKLDFREIQIYKKLRTSSFALNLPTLPEVKFPEVDVLTKYSQPEDSLIPF
FEITVPESQLTVSQFTLPKSVSDGIAALDLNAVANKIADFELPTIIVPEQTIEIPSIKFSVPAGIVIPSFQALTARFEVDSPVYN
ATWSASLKNKADYVETVLDSTCSSTVQFLEYELNVLGTHKIEDGTLASKTKGTFAHRDFSAEYEEDGKYEGLQEWEGKA
HLNIKSPAFTDLHLRYQKDKKGISTSAASPAVGTVGMDMDEDDDFSKWNFYYSPQSSPDKKLTIFKTELRVRESDEETQI
KVNWEEEAASGLLTSLKDNVPKATGVLYDYVNKYHWEHTGLTLREVSSKLRRNLQNNAEWVYQGAIRQIDDIDVRFQK
AASGTTGTYQEWKDKAQNLYQELLTQEGQASFQGLKDNVFDGLVRVTQEFHMKVKHLIDSLIDFLNFPRFQFPGKPGI
YTREELCTMFIREVGTVLSQVYSKVHNGSEILFSYFQDLVITLPFELRKHKLIDVISMYRELLKDLSKEAQEVFKAIQSLKTTE
VLRNLQDLLQFIFQLIEDNIKQLKEMKFTYLINYIQDEINTIFSDYIPYVFKLLKENLCLNLHKFNEFIQNELQEASQELQQIH
QYIMALREEYFDPSIVGWTVKYYELEEKIVSLIKNLLVALKDFHSEYIVSASNFTSQLSSQVEQFLHRNIQEYLSILTDPDGK
GKEKIAELSATAQEIIKSQAIATKKIISDYHQQFRYKLQDFSDQLSDYYEKFIAESKRLIDLSIQNYHTFLIYITELLKKLQSTTV
MNPYMKLAPGELTIIL
SEQ ID NO. 17: human Apolipoprotein C-I (ApoC-I, where Apo C-I is residues 27-83 and
truncated Apo C-I residues 29-83 in the sequence below)
>sp|P02654|APOC1_HUMAN Apolipoprotein C-I OS = Homo sapiens GN = APOC1 PE = 1 SV = 1
MRLFLSLPVLVVVLSIVLEGPAPAQGTPDVSSALDKLKEFGNTLEDKARELISRIKQSELSAKMREWFSETFQKVKEKLKIDS
SEQ ID NO. 18: human Apolipoprotein C-II (ApoC-II, which is residues 23-101 in the sequence below)
>sp|P02655|APOC2_HUMAN Apolipoprotein C-II OS = Homo sapiens GN = APOC2 PE = 1 SV = 1
MGTRLLPALFLVLLVLGFEVQGTQQPQQDEMPSPTFLTQVKESLSSYWESAKTAAQNLYEKTYLPAVDEKLRDLYSKST
AAMSTYTGIFTDQVLSVLKGEE
SEQ ID NO. 19: human Apolipoprotein C-III (ApoC-III, which is residues 21-99 in the sequence below)
>sp|P02656|APOC3_HUMAN Apolipoprotein C-III OS = Homo sapiens GN = APOC3 PE = 1 SV = 1
MQPRVLLVVALLALLASARASEAEDASLLSFMQGYMKHATKTAKDALSSVQESQVAQQARGWVTDGFSSLKDYWST
VKDKFSEFWDLDPEVRPTSAVAA
SEQ ID NO. 20: human Apolipoprotein D (ApoD, which is residues 21-189 in the sequence below)
>sp|P05090|APOD_HUMAN Apolipoprotein D OS = Homo sapiens GN = APOD PE = 1 SV = 1
MVMLLLLLSALAGLFGAAEGQAFHLGKCPNPPVQENFDVNKYLGRWYEIEKIPTTFENGRCIQANYSLMENGKIKVLN
QELRADGTVNQIEGEATPVNLTEPAKLEVKFSWFMPSAPYWILATDYENYALVYSCTCIIQLFHVDFAWILARNPNLPPE
TVDSLKNILTSNNIDVKKMTVTDQVNCPKLS
SEQ ID NO. 21: human Apolipoprotein E (ApoE, which is residues 19-317 in the sequence below)
>sp|P02649|APOE_HUMAN Apolipoprotein E OS = Homo sapiens GN = APOE PE = 1 SV = 1
MKVLWAALLVTFLAGCQAKVEQAVETEPEPELRQQTEWQSGQRWELALGRFWDYLRWVQTLSEQVQEELLSSQVT
QELRALMDETMKELKAYKSELEEQLTPVAEETRARLSKELQAAQARLGADMEDVCGRLVQYRGEVQAMLGQSTEELR
VRLASHLRKLRKRLLRDADDLQKRLAVYQAGAREGAERGLSAIRERLGPLVEQGRVRAATVGSLAGQPLQERAQAWGE
RLRARMEEMGSRTRDRLDEVKEQVAEVRAKLEEQAQQIRLQAEAFQARLKSWFEPLVEDMQRQWAGLVEKVQAAV
GTSAAPVPSDNH
SEQ ID NO. 22: human Apolipoprotein J (ApoJ isoform 1, which is residues 23-499 in the sequence
below, and where isoforms 2-5 are also available in UniProt entry P10909)
>sp|P10909|CLUS_HUMAN Clusterin OS = Homo sapiens GN = CLU PE = 1 SV = 1 (isoform 1)
MMKTLLLFVGLLLTWESGQVLGDQTVSDNELQEMSNQGSKYVNKEIQNAVNGVKQIKTLIEKTNEERKTLLSNLEEAKK
KKEDALNETRESETKLKELPGVCNETMMALWEECKPCLKQTCMKFYARVCRSGSGLVGRQLEEFLNQSSPFYFWMNG
DRIDSLLENDRQQTHMLDVMQDHFSRASSIIDELFQDRFFTREPQDTYHYLPFSLPHRRPHFFFPKSRIVRSLMPFSPYEP
LNFHAMFQPFLEMIHEAQQAMDIHFHSPAFQHPPTEFIREGDDDRTVCREIRHNSTGCLRMKDQCDKCREILSVDCST
NNPSQAKLRRELDESLQVAERLTRKYNELLKSYQWKMLNTSSLLEQLNEQFNWVSRLANLTQGEDQYYLRVTTVASHT
SDSDVPSGVTEVVVKLFDSDPITVTVPVEVSRKNPKFMETVAEKALQEYRKKHREE
SEQ ID NO. 23: human Apolipoprotein H (ApoH, which is residues 20-345 in the sequence below)
>sp|P02749|APOH_HUMAN Beta-2-glycoprotein 1 OS = Homo sapiens GN = APOH PE = 1 SV = 3
MISPVLILFSSFLCHVAIAGRTCPKPDDLPFSTVVPLKTFYEPGEEITYSCKPGYVSRGGMRKFICPLTGLWPINTLKCTPRV
CPFAGILENGAVRYTTFEYPNTISFSCNTGFYLNGADSAKCTEEGKWSPELPVCAPIICPPPSIPTFATLRVYKPSAGNNSL
YRDTAVFECLPQHAMFGNDTITCTTHGNWTKLPECREVKCPFPSRPDNGFVNYPAKPTLYYKDKATFGCHDGYSLDGP
EEIECTKLGNWSAMPSCKASCKVPVKKATVVYQGERVKIQEKFKNGMLHGDKVSFFCKNKEKKCSYTEDAQCIDGTIEV
PKCFKEHSSLAFWKTDASDVKPC
SEQ ID NO. 24: LCAT (lecithin: cholesterol acyltransferase)
MGPPGSPWQWVTLLLGLLLPPAAPFWLLNVLFPPHTTPKAELSNHTRPVILVPGCLGNQLEAKLDKPDVVNWMCYRK
TEDFFTIWLDLNMFLPLGVDCWIDNTRVVYNRSSGLVSNAPGVQIRVPGFGKTYSVEYLDSSKLAGYLHTLVQNLVNNG
YVRDETVRAAPYDWRLEPGQQEEYYRKLAGLVEEMHAAYGKPVFLIGHSLGCLHLLYELLRQPQAWKDRFIDGFISLGA
PWGGSIKPMLVLASGDNQGIPIMSSIKLKEEQRITTTSPWMFPSRMAWPEDHVFISTPSFNYTGRDFQRFFADLHFEE
GWYMWLQSRDLLAGLPAPGVEVYCLYGVGLPTPRTYIYDHGFPYTDPVGVLYEDGDDTVATRSTELCGLWQGRQPQ
PVHLLPLHGIQHLNMVFSNLTLEHINAILLGAYRQGPPASPTASPEPPPPE
SEQ ID NO. 25: CETP (cholesteryl ester transfer protein)
MLAATVLTLALLGNAHACSKGTSHEAGIVCRITKPALLVLNHETAKVIQTAFQRASYPDITGEKAMMLLGQVKYGLHNI
QISHLSIASSQVELVEAKSIDVSIQNVSVVFKGTLKYGYTTAWWLGIDOSIDFEIDSAIDLQINTQLTCDSGRVRTDAPDCY
LSFHKLLLHLQGEREPGWIKQLFTNFISFTLKLVLKGQICKEINVISNIMADFVQTRAASILSDGDIGVDISLTGDPVITASYL
ESHHKGHFIYKNVSEDLPLPTFSPTLLGDSRMLYFWFSERVFHSLAKVAFQDGRLMLSLMGDEFKAVLETWGENTNQEI
FQEVVGGFPSQAQVTVHCLKMPKISCQNKGVVVNSSVMVKFLFPRPDQQHSVAYTFEEDIVTTVQASYSKKKLFLSLLD
FQITPKTVSNLTESSSESVQSFLQSMITAVGIPEVMSRLEVVFTALMNSKGVSLFDIINPEIITRDGFLLLQMDFGFPEHLL
VDFLQSLS
SEQ ID NO. 26: PLTP (phospholipid transfer protein, variant a)
MALFGALFLALLAGAHAEFPGCKIRVTSKALELVKQEGLRFLEQELETITIPDLRGKEGHFYYNISEVKVTELQLTSSELDFQ
PQQELMLQITNASLGLRFRRQLLYWFFYDGGYINASAEGVSIRTGLELSRDPAGRMKVSNVSCQASVSRMHAAFGGTF
KKVYDFLSTFITSGMRFLLNQQICPVLYHAGTVLLNSLLDTVPVRSSVDELVGIDYSLMKDPVASTSNLDMDFRGAFFPLT
ERNWSLPNRAVEPQLQEEERMVYVAFSEFFFDSAMESYFRAGALQLLLVGDKVPHDLDMLLRATYFGSIVLLSPAVIDS
PLKLELRVLAPPRCTIKPSGTTISVTASVTIALVPPDQPEVQLSSMTMDARLSAKMALRGKALRTQLDLRRFRIYSNHSAL
ESLALIPLQAPLKTMLQIGVMPMLNERTWRGVQIPLPEGINFVHEVVTNHAGFLTIGADLHFAKGLREVIEKNRPADVR
ASTAPTPSTAAV
SEQ ID NO. 27: PON (paraoxonase) (SEQ ID NO. 27)
MAKLIALTLLGMGLALFRNHQSSYQTRLNALREVQPVELPNCNLVKGIETGSEDLEILPNGLAFISSGLKYPGIKSFNPNSP
GKILLMDLNEEDPTVLELGITGSKFDVSSFNPHGISTFTDEDNAMYLLVVNHPDAKSTVELFKFQEEEKSLLHLKTIRHKLL
PNLNDIVAVGPEHFYGTNDHYFLDPYLQSWEMYLGLAWSYVVYYSPSEVRVVAEGFDFANGINISPDGKYVYIAELLAH
KIHVYEKHANWTLTPLKSLDFNTLVDNISVDPETGDLWVGCHPNGMKIFFYDSENPPASEVLRIQNILTEEPKVTQVYAE
NGTVLQGSTVASVYKGKLLIGTVFHKALYCEL
SEQ ID NO. 28: Natural variant 3 P to H in Munster-3C. VAR_000605
DEHPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 29: Natural variant 3 P to R VAR_000606
DERPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 30: Natural variant 4 P to R in Munster-3B. Ref. 48 VAR_000607
DEPRQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 31: Natural variant 10 R to L in Baltimore. [dbSNP:rs28929476]
Ref. 47 VAR_000608
DEPPQSPWDLVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 32: Natural variant 26 1 G to R in AMYLIOWA. [dbSNP:rs28931574]
Ref. 43 Ref. 44 VAR_000609
DEPPQSPWDRVKDLATVYVDVLKDSRRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 33: Natural variant 37 1 A to T VAR_025445
DEPPQSPWDRVKDLATVYVDVLKDSGRDYVSQFEGSTLGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 34: Natural variant 60 1 L to R in AMYL8. Ref. 46 VAR_000610
DEPPQSPWDRVKDLATVYVDVLKDSGRDYVSQFEGSALGKQLNLKLLDNWDSVTSTFSKRREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 35: Natural variant 68 1 T to I VAR_017017
DEPPQSPWDRVKDLATVYVDVLKDSGRDYVSQFEGSALGKQLNLKLLDNWDSVTSTFSKLREQLGPVIQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 36: Natural variant 89 1 D to E VAR_000611
DEPPQSPWDRVKDLATVYVDVLKDSGRDYVSQFEGSALGKQLNLKLLDNWDSVTSTFSKLREQLGPVTQE
FWDNLEKETEGLRQEMSKELEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 37: Natural variant 95 1 A to D in Hita. VAR_000612
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKDKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 38: Natural variant 102 1 D to H. [dbSNP:rs5077] VAR_016189
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLHDFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 39: Natural variant 103 1 D to N in Munster-3A. VAR_000613
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDNFQKKWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 40: Natural variant 107 1 K to M. [dbSNP:rs4882]
Ref. 49 VAR_000615
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKMWQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 41: Natural variant 107 1 (Lys107d) Missing in Marburg/Munster-2
(Helsinki). VAR_000614
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKWQEEMELYRQKVEPLRAELQEGARQKLHELQEKL
SPLGEEMRDRARAHVDALRTHLAPYSDELRQRLAARLEALKENGGARLAEYHAKATEHLSTLSEKAKPAL
EDLRQGLLPVLESFKVSFLSALEEYTKKLNT
SEQ ID NO. 42: Natural variant 108 1 W to R in Tsushima. VAR_000616
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKRQEEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 43: Natural variant 110 1 E to K in Fukuoka. Ref. 45 VAR_000617
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQKEMELYRQKVEPLRAELQEGARQKLHELQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 44: Natural variant 126 1 E to K in Norway. Ref. 42 VAR_000618
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHKLQEK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 45: Natural variant 139 1 E to G VAR_000619
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQGK
LSPLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 46: Natural variant 143 1 P to R in Giessen. Ref. 41 VAR_000620
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSRLGEEMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 48: Natural variant 147 1 E to V VAR_000622
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEVMRDRARAHVDALRTHLAPYSDELRQRLAARLEALKENGGARLAEYHAKATEHLSTLSEKAKPA
LEDLRQGLLPVLESFKVSFLSALEEYTKKLNT
SEQ ID NO. 49: Natural variant 156 1 V to E in Oita; 60% of normal apoA-I and normal
HDL cholesterol levels. Rapidly cleared from plasma. Ref. 51 VAR_021362
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHEDALRTHLAPYSDELRQRLAARLEALKENGGARLAEYHAKATEHLSTLSEKAKPA
LEDLRQGLLPVLESFKVSFLSALEEYTKKLNT
SEQ ID NO. 50: Natural variant 159 L to P in Zavalla
DEPPQSPWDRVKDLATVYVDVLKDSGRDYVSQFEGSALGKQLNLKLLDNWDSVTSTESKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDAPRTHLAPYSDELRQRLAARLEALKENGGARLAEYHAKATEHLSTLSEKAKPA
LEDLRQGLLPVLESFKVSFLSALEEYTKKLNT
SEQ ID NO. 51: Natural variant 160 1 R to P. [dbSNP:rs5078] VAR_014609
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALPTHLAPYSDELRQRLAARLEALKENGGARLAEYHAKATEHLSTLSEKAKPA
LEDLRQGLLPVLESFKVSFLSALEEYTKKLNT
SEQ ID NO. 52: Natural variant 165 1 P to R VAR_000623
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLARYSDELRQRLAARLEALKENGGARLAEYHAKATEHLSTLSEKAKPA
LEDLRQGLLPVLESFKVSFLSALEEYTKKLNT
SEQ ID NO. 53: Natural variant 198 1 E to K in Munster-4. Ref. 49 VAR_000625
DEPPQSPWDRVKDLATVYVDVLKDSGRDYVSQFEGSALGKQLNLKLLDNWDSVTSTFSKLREQLGPVTQE
FWDNLEKETEGLRQEMSKDLEEVKAKVQPYLDDFQKKWQEEMELYRQKVEPLRAELQEGARQKLHELQEK
LSPLGEEMRDRARAHVDALRTHLAPYSDELRQRLAARLEALKENGGARLAEYHAKATKHLSTLSEKAKPA
LEDLRQGLLPVLESFKVSFLSALEEYTKKLNT

Lipoprotein complexes for use in the present invention comprise a lipid fraction containing neutral and charged phospholipids and have the following features: contain neutral phospholipids selected from lecithin and spingomyelin or a combination thereof, at a ratio of about 0.2 to 3 wt % of the charged phospholipid, contain a combination of lecithin and spingomylin at ratio of lecithin:spingomyelin of 100:5 to 5:100; contain charged phospholipids selected from phosphatidylinositol, phosphatidylserine and phosphatidylglycerol, phosphitic acid or a combination thereof having an acyl chain length of between 6 to 24 carbons; contain lipid and apolipoprotein at a ratio of 20:1 to 60:1 and preferably 50:1; contain 2-4 protein molecules per 200-400 molecules of neutral phospholipid and per 1 molecule of charged phospholipid. Where spingomyelin is included in the lipid fraction D-erythrose-sphingomyelin, D-erythrose-dihydrosphingomyclin or mixtures thereof can be used. Lecithin is selected from POPC DPPC or a mixture thereof. In one embodiment the apolipoprotein complex contains charged and neutral lipids as specified above and Human Apo A-I (SEQ ID NO. 3), Apo A-I Milano (SEQ ID No. 11) or a peptide analogue of Apo A-I (i.e., SEQ ID NO. 54-165) at a ratio of 2-4 protein molecules per 200-400 molecules of neutral phospholipid and at a ratio of 2-4 protein molecules per molecule of charged phospolipid. US application US 2006/0217312 is hereby incorporated by reference.

Apolipoprotein complexes, comprising a ApoA-I apolipoprotein selected from mature human ApoA-I (SEQ ID NO. 3) apolipoprotein, mature ApoA-I Milano (SEQ ID NO. 11), mature ApoA-I Paris (SEQ ID NO. 10), and mixtures thereof may contain multiple types of phospholipids in the lipid fraction of the apolipoprotein complex including but not limited to one of more phospholipids selected from, sphingomyelin (SPH), 1,2-dipalmitoyl-sn-glycero-3-phosphocholine (DPPC) and 1,2-dipalmitoyl-sn-glycero-3-[phospho-rac-(1-glycerol)] (DPPG). Preferably the lipid composition of the apolipoprotein complex is 48.5% SPH/48.5% DPPC/3% DPPG (w/w/w).

Apolipoprotein complexes comprising a ApoA-I apolipoprotein selected from mature human ApoA-I (SEQ ID NO. 3) apolipoprotein, mature ApoA-I Milano (SEQ ID NO. 11), mature ApoA-I Paris (SEQ ID NO. 10), and mixtures thereof may contain essentially sphingomyelin in the lipid fraction in combination with about 3% wt/wt of a negatively charged phospholipid selected from phosphatidylinositol, phosphatidylserine, phosphatidylglycerol, phosphatidic acid, and mixtures thereof. Either D-erythrose-sphingomyelin and/or D-erythrose dihydrosphingomyelin or any combination thereof can be used as the neutral amino acid. The acyl chains of the sphingomyelin or other negatively charged phospholipids in the lipid phase are selected from a saturated, a mono-unsaturated and a polyunsaturated hydrocarbon containing from 6 to 24 carbon atoms and may differ in the degree of saturation.

Apolipoprotein complexes comprising a ApoA-I apolipoprotein selected from mature human ApoA-I (SEQ ID NO. 3) apolipoprotein, mature ApoA-I Milano (SEQ ID NO. 11), mature ApoA-I Paris (SEQ ID NO. 10) and mixtures thereof with an apolipoprotein and lipid at a ratio in the range of about 1:100 to 1:200 and preferably 1:30 to 1:100.

Apolipoprotein complexes for use in the present invention include those where the protein fraction comprises an apolipoprotein A-I analogue (Apo A-I analogue). In one embodiment the Apo A-I analogue is a peptide of 15 to 29-amino acid residues, according to formula 1 below, which forms an amphipathic α-helix in the presence of lipids. Apo A-I analogue peptides for use in the present invention include peptides of 15 to 29 amino acid residues according to the Formula 1 wherein,

Z₁-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-Z₂₄ Formula 1

X₁ is Pro (P), Ala (A), Gly (G), Gln (Q), Asn (N), Asp (D) or D-Pro (p); X₂ is an aliphatic residue; X₃ is Leu (L) or Phe (F); X₄ is an acidic residue; X₅ is Leu (L) or Phe (F); X₆ is Leu (L) or Phe (F); X₇ is a hydrophilic residue; X₈ is an acidic or a basic residue; X₉ is Leu (L) or Gly (G); X₁₀ is Leu (L), Trp (W) or Gly (G); X₁₁ is a hydrophilic residue; X₁₂ is a hydrophilic residue; X₁₃ is Gly (G) or an aliphatic residue; X₁₄ is Leu (L), Trp (W), Gly (G) or Nal; X₁₅ is a hydrophilic residue; X₁₆ is a hydrophobic residue; X₁₇ is a hydrophobic residue; X₁₈ is Gln (Q), Asn (N) or a basic residue; X₁₉ is Gln (Q), Asn (N) or a basic residue; X₂₀ is a basic residue; X₂₁ is an aliphatic residue; X₂₂ is a basic residue; X₂₃ is absent or a basic residue; Z₁ is H₂N— or RC(O)NH—; and Z₂ is —C(O)NRR, —C(O)OR or —C(O)OH or a salt thereof;
R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and
each “-” between residues X₁ through X₂₃ designates an amide linkage, a substituted amide linkage, an isostere of an amide or an amide mimetic.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₁ is Pro (P), D-Pro (p), Gly (G) or Ala (A); X₂ is Ala (A), Leu (L) or Val (V); X₃ is Leu (L) or Phe (F); X₅ is Leu (L) or Phe (F); X₆ is Leu (L) or Phe (F); X₉ is Leu (L) or Gly (G); X₁₀ is Leu (L), Trp (W) or Gly (G); X₁₃ is Leu (L), Gly (G) or Aib; X₁₄ is Leu, Nal, Trp (W) or Gly (G); X₁₆ is Ala (A), Nal, Trp (W), Gly (G), Leu (L) or Phe (F); X₁₇ is Leu (L), Gly (G) or Nal; X₂₁ is Leu (L); X₄ is an acidic residue; X₇ is a hydrophilic residue; X₈ is an acidic or a basic residue; X₁₁ is a hydrophilic residue; X₁₂ is a hydrophilic residue; X₁₅ is a hydrophilic residue; X₁₈ is Gln (Q), Asn (N) or a basic residue; X₁₉ is Gln (Q), Asn (N) or a basic residue; X₂₀ is a basic residue; X₂₂ is a basic residue; X₂₃ is absent or a basic residue; Z₁ is H₂N— or RC(O)NH—; and Z₂ is —C(O)NRR, —C(O)OR or —C(O)OH or a salt thereof;
R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₆-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and
wherein each “-” between residues X₁ through X₂₃ designates an amide linkage, a substituted amide linkage, an isostere of an amide or an amide mimetic.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₃ is Leu (L) or Phe (F); X₄ is Asp (D) or Glu (E); X₆ is Phe (F); X₇ is Lys (K), Arg (R) or Orn; X₈ is Asp (D) or Glu (E); X₉ is Leu (L) or Gly (G); X₁₀ is Leu (L) or Trp (W) or Gly (G); X₁₁ is Asn (N) or Gln (Q); X₁₂ is Glu (E) or Asp (D); X₁₅ is Asp (D) or Glu (E); Xᵢₛ is Gln (Q), Asn (N), Lys (K) or Orn; X₁₉ is Gln (Q), Asn (N), Lys (K) or Orn; X₂₀ is Lys (K) or Orn; X₂₂ is Lys (K) or Orn; X₂₃ is absent or Lys (K); X₁ is Pro (P), Ala (A), Gly (G), Gln (Q), Asn (N), Asp (D) or D-Pro (p); X₂ is an aliphatic residue; X₃ is Leu (L) or Phe (F); X₅ is Leu (L) or Phe (F); X₆ is Leu (L) or Phe (F); X₉ is Leu (L) or Gly (G); X₁₀ is Leu (L), Trp (W) or Gly (G); X₁₃ is Gly (G) or an aliphatic residue; X₁₄ is Leu (L), Trp (W), Gly (G) or Nal; X₁₆ is a hydrophobic residue; X₁₇ is a hydrophobic residue; X₂₁ is an aliphatic residue; Z₁ is H₂N— or RC(O)NH—; and Z₂ is —C(O)NRR, —C(O)OR or —C(O)OH or a salt thereof;

R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and
each “-” between residues X₁ through X₂₃ designates an amide linkage, a substituted amide linkage, an isostere of an amide or an amide mimetic.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₁ is Pro (P), Ala (A), Gly (Q), Gln (Q), Asn (N), Asp (D) or D-Pro (p); X₂ is an aliphatic residue; X₃ is Leu (L) or Phe (F); X₄ is an acidic residue; X₅ is Leu (L) or Phe (F); X₆ is Leu (L) or Phe (F); X₇ is a hydrophilic residue; X₈ is an acidic or a basic residue; X₉ is Leu (L) or Gly (G); X₁₀ is Leu (L), Trp (W) or Gly (G); X₁₁ is a hydrophilic residue; X₁₂ is a hydrophilic residue; X₁₃ is Gly (G) or an aliphatic residue; X₁₄ is Leu (L), Trp (W), Gly (G) or Nal; X₁₅ is a hydrophilic residue; X₁₆ is a hydrophobic residue; X₁₇ is a hydrophobic residue; X₁₈ is Gln (Q), Asn (N) or a basic residue; X₁₉ is Gln (Q), Asn (N) or a basic residue; X₂₀ is a basic residue; X₂₁ is an aliphatic residue; X₂₂ is a basic residue; X₂₃ is absent or a basic residue; Z₁ is H₂N— or RC(O)NH—; Z₂ is —C(O)NRR, —C(O)OR or —C(O)OH or a salt thereof;

R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and

each “-” between residues X₁ through X₂₃ designates an amide linkage, a substituted amide linkage, an isostere of an amide or an amide mimetic.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₁ is Pro (P), Ala (A), Gly (G), Gin (Q), Asn (N), Asp (D) or D-Pro (p); X₂ is an aliphatic residue; X₃ is Leu (L) or Phe (F); X₄ is an acidic residue; X₅ is Leu (L) or Phe (F); X₆ is Leu (L) or Phe (F); X₇ is a hydrophilic residue; X₈ is an acidic or a basic residue; X₉ is Leu (L) or Gly (G); X₁₀ is Leu (L), Trp (W) or Gly (G); X₁₁ is a hydrophilic residue; X₁₂ is a hydrophilic residue; X₁₃ is Gly (G) or an aliphatic residue; X₁₄ is Leu (L), Trp (W), Gly (G) or Nal; X₁₅ is a hydrophilic residue; X₁₆ is a hydrophobic residue; X₁₇ is a hydrophobic residue; X₁₈ is Gln (Q), Asn (N) or a basic residue; X₁₉ is Gln (Q), Asn (N) or a basic residue; X₂₀ is a basic residue; X₂₁ is an aliphatic residue; X₂₂ is a basic residue; X₂₃ is absent or a basic residue; Z₁ is H₂N—; Z₂ is —C(O)OR or a salt thereof;
R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and each “-” between residues X₁ through X₂₃ designates —C(O)NH—.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₁ is Pro (P), Ala (A), Gly (G), Asn (N), Gln (Q), Asp (D) or D-Pro (p); X₂ is Ala (A), Val (V) or Leu (L); X₃ is Leu (L) or Phe (F); X₄ is Asp (D) or Glu (E); X₅ is Leu (L) or Phe (F); X₆ is Leu (L) or Phe (F); X₇ is Lys (K), Arg (R) or Orn; X₈ is Asp (D) or Glu (E); X₉ is Leu (L) or Gly (G); X₁₀ is Leu (L), Trp (W) or Gly (G); X₁₁ is Asn (N) or Gln (Q); X₁₂ is Glu (E) or Asp (D); X₁₃ is Gly (G), Leu (L) or Aib; X₁₄ is Leu (L), Nal, Trp (W) or Gly (G); X₁₅ is Asp (D) or Glu (E); X₁₆ is Ala (A), Nal, Trp (W), Leu (L), Phe (F) or Gly (G); X₁₇ is Gly (G), Leu (L) or Nal; X₁₈ is Gln (Q), Asn (N), Lys (K) or Orn; X₁₉ is Gln (Q), Asn (N), Lys (K) or Orn; X₂₀ is Lys (K) or Orn; X₂₁ is Leu (L); X₂₂ is Lys (K) or Orn; and X₂₃ is absent or Lys (K).; Z₁ is H₂N—; Z₂ is —C(O)OR or a salt thereof;

R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and each “-” between residues X₁ through X₂₃ designates —C(O)NH—.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₁ is Pro (P), Ala (A), Gly (G), Asn (N), Gln (Q), Asp (D) or D-Pro (p); X₂ is Ala (A), Val (V) or Leu (L); X₃ is Leu (L) or Phe (F); X₄ is Asp (D) or Glu (E); X₅ is Leu (L) or Phe (F); X₆ is Leu (L) or Phe (F); X₇ is Lys (K), Arg (R) or Orn; X₈ is Asp (D) or Glu (E); X₉ is Leu (L) or Gly (G); X₁₀ is Leu (L), Trp (W) or Gly (G); X₁₁ is Asn (N) or Gln (Q); X₁₂ is Glu (E) or Asp (D); X₁₃ is Gly (G), Leu (L) or Aib; X₁₄ is Leu (L), Nal, Trp (W) or Gly (G); X₁₅ is Asp (D) or Glu (E); X₁₆ is Ala (A), Nal, Trp (W), Leu (L), Phe (F) or Gly (G); X₁₇ is Gly (G), Leu (L) or Nal; X₁₈ is Gln (Q), Asn (N), Lys (K) or Orn; X₁₉ is Gln (Q), Asn (N), Lys (K) or Orn; X₂₀ is Lys (K) or Orn; X₂₁ is Leu (L); X₂₂ is Lys (K) or Orn; and X₂₃ is absent; Z₁ is H₂N—; Z₂ is —C(O)OR or a salt thereof;
R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and each “-” between residues X₁ through X₂₂ designates —C(O)NH—.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₁ is Pro (P), Ala (A), Gly (G), Asn (N), Gln (Q), Asp (D) or D-Pro (p); X₂ is Ala (A), Val (V) or Leu (L); X₃ is Leu (L) or Phe (F); X₄ is Asp (D) or Glu (E); X₅ is Leu (L) or Phe (F); X₆ is Leu (L) or Phe (F); X₇ is Lys (K), Arg (R) or Orn; X₈ is Asp (D) or Glu (E); X₉ is Leu (L) or Gly (G); X₁₀ is Leu (L), Trp (W) or Gly (G); X₁₁ is Asn (N) or Gln (Q); X₁₂ is Glu (E) or Asp (D); X₁₃ is Gly (G), Leu (L) or Aib; X₁₄ is Leu (L), Nal, Trp (W) or Gly (G); X₁₅ is Asp (D) or Glu (E); X₁₆ is Ala (A), Nal, Trp (W), Leu (L), Phe (F) or Gly (G); X₁₇ is Gly (G), Leu (L) or Nal; X₁₈ is Gln (Q), Asn (N); X₁₉ is Gln (Q), Asn (N); X₂₀ is Lys (K) or Orn; X₂₁ is Leu (L); X₂₂ is Lys (K) or Orn; and X₂₃ is absent or Lys (K).; Z₁ is H₂N—; Z₂ is —C(O)OR or a salt thereof;
R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and each “-” between residues X₁ through X₂₃ designates —C(O)NH—.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₁ is Pro (P), Ala (A), Gly (G), Asn (N), Gln (Q), Asp (D) or D-Pro (p); X₂ is Ala (A), Val (V) or Leu (L); X₃ is Leu (L) or Phe (F); X₄ is Asp (D) or Glu (E); X₅ is Leu (L) or Phe (F); X₆ is Leu (L) or Phe (F); X₇ is Lys (K), Arg (R) or Orn; X₈ is Asp (D) or Glu (E); X₉ is Leu (L); X₁₀ is Leu (L), Trp (W); X₁₁ is Asn (N) or Gin (Q); X₁₂ is Glu (E) or Asp (D); X₁₃ is Gly (G), Leu (L) or Aib; X₁₄ is Leu (L), Nal, or Trp (W); X₁₅ is Asp (D) or Glu (E); X₁₆ is Ala (A), Nal, Trp (W), Leu (L), or Phe (F); X₁₇ is Leu (L) or Nal; X₁₈ is Gln (Q), Asn (N), Lys (K) or Orn; X₁₉ is Gln (Q), Asn (N), Lys (K) or Orn; X₂₀ is Lys (K) or Orn; X₂₁ is Leu (L); X₂₂ is Lys (K) or Orn; and X₂₃ is absent; Z₁ is H₂N—; Z₂ is —C(O)OR or a salt thereof;

R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and each “-” between residues X₁ through X₂₂ designates —C(O)NH—.

Further Apo A-I analogues for use in the present invention, as part of a apolipoprotein complex for treating LVDD, include a 15 to 29-residue peptide, which forms an amphipathic α-helix in the presence of lipids, wherein the peptide includes a peptide of Formula 1 wherein:

X₁ is Pro (P), Ala (A), Gly (G), Asn (N), Gln (Q), Asp (D) or D-Pro (p); X₂ is Ala (A), Val (V) or Leu (L); X₃ is Leu (L) or Phe (F); X₄ is Asp (D) or Glu (E); X₅ is Leu (L) or Phe (F); X₆ is Leu (L) or Phe (F); X₇ is Lys (K), Arg (R) or Orn; X₈ is Asp (D) or Glu (E); X₉ is Gly (G); X₁₀ is Gly (G); X₁₁ is Asn (N) or Gln (Q); X₁₂ is Glu (E) or Asp (D); X₁₃ is Gly (G); X₁₄ is Gly (G); X₁₅ is Asp (D) or Glu (E); X₁₆ is Gly (G); X₁₇ is Gly (G); X₁₈ is Gln (Q), Asn (N), Lys (K) or Orn; X₁₉ is Gln (Q), Asn (N), Lys (K) or Orn; X₂₀ is Lys (K) or Orn; X₂₁ is Leu (L); X₂₂ is Lys (K) or Orn; and X₂₃ is absent; Z₁ is H₂N—; Z₂ is —C(O)OR or a salt thereof;

R is selected from the group consisting of H, (C₁-C₆) alkyl, (C₂-C₆) alkenyl, (C₂-C₆) alkynyl, (C₅-C₂₀) aryl, (C₆-C₂₆) alkaryl, (C₅-C₂₀) heteroaryl, (C₆-C₂₆) alkheteroaryl, and a 1 to 7-residue peptide wherein one or more bonds between residues 1-7 is a substituted amide, an isostere of an amide or an amide mimetic; and each “-” between residues X₁ through X₂₂ designates —C(O)NH—.

Further Apo A-I analogues for use in the present invention, as part of a apolipoprotein complex for treating LVDD, include a 15 to 29-residue peptide, which forms an amphipathic α-helix in the presence of lipids, selected from the group consisting of:

(SEQ ID NO. 54)
GVLDLFRELLNELLEALKQKLKK
(SEQ ID NO. 55)
PVLDLFRELLNELLEWLKQKLK
(SEQ ID NO. 56)
PVLDLFRELLNELLEALKQKLK
(SEQ ID NO. 57)
PVLDLFRELLNELLEALKQKLKK
(SEQ ID NO. 58)
PVLDLFRELLNEXLEALKQKLK
(SEQ ID NO. 59)
PVLDLFKELLNELLEALKQKLK
(SEQ ID NO. 60)
PVLDLFRELLNEGLEALKQKLK
(SEQ ID NO. 61)
PVLDLFRELGNELLEALKQKLK
(SEQ ID NO. 62)
PVLDLFRELLNELLEAZKQKLK
(SEQ ID NO. 63)
PVLDLFKELLQELLEALKQKLK
(SEQ ID NO. 64)
PVLDLFRELLNELLEAGKQKLK
(SEQ ID NO. 65)
GVLDLFRELLNEGLEALKQKLK
(SEQ ID NO. 66)
PVLDLFRELLNELLEALOQOLO
(SEQ ID NO. 67)
PVLDLFRELWNELLEALKQKLK
(SEQ ID NO. 68)
PVLDLLRELLNELLEALKQKLK
(SEQ ID NO. 69)
PVLELFKELLQELLEALKQKLK
(SEQ ID NO. 70)
GVLDLFRELLNELLEALKQKLK
(SEQ ID NO. 71)
PVLDLFRELLNEGLEALKQKLK
(SEQ ID NO. 72)
PVLDLFREGLNELLEALKQKLK
(SEQ ID NO. 73)
PVLDLFRELLNELLEALKQKLK
(SEQ ID NO. 74)
PVLDLFRELLNELLEGLKQKLK
(SEQ ID NO. 75)
PLLELFKELLQELLEALKQKLK
(SEQ ID NO. 76)
PVLDLFRELLNELLEALQKKLK
(SEQ ID NO. 77)
PVLDFFRELLNEXLEALKQKLK
(SEQ ID NO. 78)
PVLDLFRELLNELLELLKQKLK
(SEQ ID NO. 79)
PVLDLFRELLNELZEALKQKLK
(SEQ ID NO. 80)
PVLDLFRELLNELWEALKQKLK
(SEQ ID NO. 81)
AVLDLFRELLNELLEALKQKLK
(SEQ ID NO. 82)
QVLDLFRELLNELLEALKQKLK
(SEQ ID NO. 83)
PVLDLFOELLNELLEALOQOLO
(SEQ ID NO. 84)
NVLDLFRELLNELLEALKQKLK
(SEQ ID NO. 85)
PVLDLFRELLNELGEALKQKLK
(SEQ ID NO. 86)
PVLDLFRELLNELLELLKQKLK
(SEQ ID NO. 87)
PVLDLFRELLNELLEFLKQKLK
(SEQ ID NO. 88)
PVLELFNDLLRELLEALQKKLK
(SEQ ID NO. 89)
PVLELFNDLLRELLEALKQKLK
(SEQ ID NO. 90)
PVLELFKELLNELLDALRQKLK
(SEQ ID NO. 91)
PVLDLFRELLENLLEALQKKLK
(SEQ ID NO. 92)
PVLELFERLLEDLLQALNKKLK
(SEQ ID NO. 93)
PVLELFERLLEDLLKALNQKLK
(SEQ ID NO. 94)
DVLDLFRELLNELLEALKQKLK
(SEQ ID NO. 95)
PALELFKDLLQELLEALKQKLK
(SEQ ID NO. 96)
PVLDLFRELLNEGLEAZKQKLK
(SEQ ID NO. 97)
PVLDLFRELLNEGLEWLKQKLK
(SEQ ID NO. 98)
PVLDLFRELWNEGLEALKQKLK
(SEQ ID NO. 99)
PVLDLFRELLNEGLEALOQOLO
(SEQ ID NO. 100)
PVLDFFRELLNEGLEALQKKLK
and
(SEQ ID NO. 101)
PVLELFRELLNEGLEALKQKLK;

including N-terminal acylated, C-terminal amidated and esterified forms thereof.

Other Apo A-I analogues for use in the present invention, as part of a apolipoprotein complex for treating diastolic dysfunction, include a 15 to 29-residue peptide, which forms an amphipathic α-helix in the presence of lipids and comprises SEQ ID NO. 56.

One example of an Apo A-I analogue for use in the present invention, as part of a apolipoprotein complex for treating diastolic dysfunction, includes a peptide consisting of SEQ ID NO. 56.

Other Apo A-I analogues for use in the present invention include a 22 to 29 residue peptide according to Formula 2 wherein:

R¹-Y¹-X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-Y²-R² (Formula 2), wherein

X¹ is absent or a basic achiral amino acid residue, a basic D-amino acid residue, or a basic L-amino acid residue; X² is a basic achiral amino acid residue, a basic D-amino acid residue, or a basic L-amino acid residue; X³ is an aliphatic achiral amino acid residue, an aliphatic D-amino acid residue, or an aliphatic L-amino acid residue; X⁴ is a basic achiral amino acid residue, a basic D-amino acid residue, or a basic L-amino acid residue; X⁵ is Gln, Asn, D-Gln, D-Asn, or a basic achiral amino acid residue, a basic D-amino acid residue, or a basic L-amino acid residue; X⁶ is a basic achiral amino acid residue, a basic D-amino acid residue, or a basic L-amino acid residue; X⁷ is a hydrophobic achiral amino acid residue, a hydrophobic D-amino acid residue, or a hydrophobic L-amino acid residue; X⁸ is a hydrophobic achiral amino acid residue, a hydrophobic D-amino acid residue, or a hydrophobic L-amino acid residue; X⁹ is a hydrophilic achiral amino acid residue, a hydrophilic D-amino acid residue, or a hydrophilic L-amino acid residue; X¹⁰ is Leu, Trp, Gly, Nal, D-Leu, D-Trp, or D-Nal; X¹¹ is Gly or an aliphatic achiral amino acid residue, an aliphatic D-amino acid residue, or an aliphatic L-amino acid residue; X¹² is a hydrophilic achiral amino acid residue, a hydrophilic D-amino acid residue, or a hydrophilic L-amino acid residue; X¹³ is a hydrophilic achiral amino acid residue, a hydrophilic D-amino acid residue, or a hydrophilic L-amino acid residue; X¹⁴ is Leu, Trp, Gly, D-Leu, or D-Trp; X¹⁵ is Leu, Gly, or D-Leu; X¹⁶ is an acidic achiral amino acid residue, an acidic D-amino acid residue, or an acidic L-amino acid residue; X¹⁷ is a hydrophilic achiral amino acid residue, a hydrophilic D-amino acid residue, or a hydrophilic L-amino acid residue; X¹⁸ is Leu, Phe, D-Leu, or D-Phe; X¹⁹ is Leu, Phe, D-Leu, or D-Phe; X²⁰ is an acidic achiral amino acid residue, an acidic D-amino acid residue, or an acidic L-amino acid residue; X²¹ is Leu, Phe, D-Leu, or D-Phe; X²² is an aliphatic achiral amino acid residue, an aliphatic D-amino acid residue, or an aliphatic L-amino acid residue; and X²³ is Inp, Nip, azPro, Pip, azPip, D-Nip, or D-Pip;
Y¹ is absent or a sequence of 1 to 7 amino acid residues, wherein each residue of the sequence is independently an achiral, D-, or L-amino acid residue;

Y² is absent or a sequence of 1 to 7 amino acid residues, wherein each residue of the sequence is independently an achiral, D-, or L-amino acid residue;

R¹ is H or an amino protecting group; and R² is OH or a carboxyl protecting group; and wherein: (a) all amino acid residues, other than the terminal amino acid residues and residues immediately adjacent to the terminal amino acid residues, are achiral or L-amino acid residues; or (b) all amino acid residues, other than the terminal amino acid residues and residues immediately adjacent to the terminal amino acid residues, are achiral or D-amino acid residues.

Other Apo A-I analogues for use in the present invention a 22- or 23-residue peptide according to Formula 2 as described in paragraph [00137] above wherein:

X³ is Leu or D-Leu; X⁷ is Leu, Gly, Nal, D-Leu, or D-Nal; X⁸ is Ala, Nal, Trp, Gly, Leu, Phe, D-Ala, D-Nal, D-Trp, D-Leu, or D-Phe; X¹¹ is Leu, Gly, Aib, or D-Leu; and X²² is Ala, Leu, Val, D-Ala, D-Leu, or D-Val.

Other Apo A-I analogues for use in the present invention a 22- or 23-residue peptide according to Formula 2 as described in the paragraph [00137] above wherein:

X¹ is absent, Lys, or D-Lys; X² is Lys, Orn, D-Lys, or D-Orn; X⁴ is Lys, Orn, D-Lys, or D-Orn; X⁵ is Gln, Asn, Lys, Orn, D-Gln, D-Asn, D-Lys, or D-Orn; X⁶ is Gln, Asn, Lys, Orn, D-Gln, D-Asn, D-Lys, or D-Orn; X⁹ is Asp, Glu, D-Asp, or D-Glu; X¹² is Glu, Asp, D-Asp, or D-Glu; X¹³ is Asn, Gln, D-Asn or D-Gln; X¹⁶ is Asp, Glu, D-Asp, or D-Glu; X¹⁷ is Lys, Arg, Orn, D-Lys, D-Arg, or D-Orn; X²⁰ is Asp, Glu, D-Asp, or D-Glu; X¹⁸ is Phe or D-Phe; and R¹ is H and R² is OH.

Other Apo A-I analogues for use in the present invention a 22- or 23-residue peptide according to Formula 2 as described in the paragraph [00137] above wherein:

X¹ is absent, Lys or D-Lys; X² is Lys, Orn, D-Lys, or D-Orn; X³ is Leu or D-Leu; X⁴ is Lys, Urn, D-Lys, or D-Orn; X⁵ is Gln, Asn, Lys, Orn, D-Gln, D-Asn, D-Lys, or D-Orn; X⁶ is Lys, Orn, D-Lys, or D-Orn; X⁷ is Gly, Leu, Nal, D-Leu, or D-Nal; X⁸ is Ala, Nal, Trp, Leu, Phe, Gly, D-Ala, D-Nal, D-Trp, D-Leu, or D-Phe; X⁹ is Asp, Glu, D-Asp, or D-Glu; X¹¹ is Gly, Leu, Aib, or D-Leu; X¹² is Glu, Asp, D-Glu, or D-Asp; X¹³ is Asn, Gln, D-Asn, or D-Gln; X¹⁶ is Asp, Glu, D-Asp, or D-Glu; X¹⁷ is Lys, Arg, Orn, D-Lys, D-Arg, or D-Orn; X²⁰ is Asp, Glu, D-Asp, or D-Glu; X²² is Ala, Val, Leu, D-Ala, D-Val, or D-Leu; and R¹ is H and R² is OH.

Other Apo A-I analogues for use in the present invention include a 22-residue peptide according to Formula 2 as described in the paragraph [00137] above wherein:

X¹ is absent; X² and X⁴ are both Lys, Orn, D-Lys, or D-Orn; X⁵ is Gln, Lys, D-Gln, or D-Lys; X⁶ is Lys, Orn, D-Lys, or D-Orn; X⁷ is Gly, Leu, Nal, D-Leu, or D-Nal; X⁸ is Ala, Nal, Trp, Leu, Phe, Gly, D-Ala, D-Nal, D-Trp, D-Leu, or D-Phe; X⁹ is an acidic achiral amino acid residue, an acidic D-amino acid residue, or an acidic L-amino acid residue; X¹⁰ is Leu, Trp, Gly, Nal, D-Leu, D-Trp, or D-Nal; X¹¹ is Gly, Leu, Aib, or D-Leu; X¹² is Glu, Asn, Gln, Arg, D-Glu, D-Asn, D-Gln, or D-Arg; X¹³ is Glu, Asn, Gln, Arg, D-Glu, D-Asn, D-Gln, or D-Arg; X¹⁴ is Leu, Trp, Gly, D-Leu, or D-Trp; X¹⁵ is Leu, Gly, or D-Leu; X¹⁶ is an acidic achiral amino acid residue, an acidic D-amino acid residue, or an acidic L-amino acid residue; X¹⁷ is Arg, Lys, Orn, D-Arg, D-Lys, or D-Orn; X¹⁸ is Phe or D-Phe; X¹⁹ is Leu, Phe, D-Leu, or D-Phe; X²⁰ is Asp, Glu, D-Asp, or D-Glu; X²¹ is Leu or D-Leu; X²² is Ala, Val, Leu, D-Ala, D-Val, or D-Leu; and R¹ is H and R² is OH.

Other Apo A-I analogues for use in the present invention include a 22-residue peptide according to Formula 2 as described in the paragraph [00137] above wherein:

X¹ is absent; X² and X⁴ are both Lys, Orn, D-Lys, or D-Orn; X³ is Leu or D-Leu; X⁵ is Gln, Lys, D-Gln, or D-Lys; X⁶ is Lys, Orn, D-Lys, or D-Orn; X⁷ is Gly, Leu, Nal, D-Leu, or D-Nal; X⁸ is Ala, Nal, Trp, Leu, Phe, Gly, D-Ala, D-Nal, D-Trp, D-Leu, or D-Phe; X⁹ is an acidic achiral amino acid residue, an acidic D-amino acid residue, or an acidic L-amino acid residue; X¹⁰ is Leu, Trp, Gly, Nal, D-Leu, D-Trp, or D-Nal; X¹¹ is Gly, Leu, Aib, or D-Leu; X¹² is Glu, Asn, Gln, Arg, D-Glu, D-Asn, D-Gln, or D-Arg; X¹³ is Glu, Asn, Gln, Arg, D-Glu, D-Asn, D-Gln, or D-Arg; X¹⁴ is Leu, Trp, Gly, D-Leu, or D-Trp; X¹⁶ is an acidic achiral amino acid residue, an acidic D-amino acid residue, or an acidic L-amino acid residue; X¹⁷ is a hydrophilic achiral amino acid residue, a hydrophilic D-amino acid residue, or a hydrophilic L-amino acid residue; X¹⁸ is Leu, Phe, D-Leu, or D-Phe; X¹⁹ is Leu, Phe, D-Leu, or D-Phe; X²⁰ is an acidic achiral amino acid residue, an acidic D-amino acid residue, or an acidic L-amino acid residue; X²¹ is Leu, Phe, D-Leu, or D-Phe; X²² is an aliphatic achiral amino acid residue, an aliphatic D-amino acid residue, or an aliphatic L-amino acid residue; and X²³ is Inp, Nip, azPro, Pip, azPip, D-Nip, or D-Pip;

Other Apo A-I analogues for use in the present invention include a peptide selected from the group consisting of:

(SEQ ID NO. 102)
Lys-Leu-Lys-Gln-Lys-Leu-Trp-Glu-Leu-Leu-Glu-Asn-Leu-Leu-Glu-Arg-Phe-Leu-Asp-Leu-
Val-Inp
(SEQ ID NO. 103)
Lys-Leu-Lys-Gln-Lys-Leu-Ala-Glu-Leu-Gly-Glu-Asn-Leu-Leu-Glu-Arg-Phe-Leu-Asp-Leu-
Val-Inp
(SEQ ID NO. 104)
Lys-Leu-Lys-Gln-Lys-Nal-Ala-Glu-Leu-Gly-Glu-Asn-Leu-Leu-Glu-Arg-Phe-Leu-Asp-Leu-
Val-Inp
(SEQ ID NO. 105)
Lys-Leu-Lys-Gln-Lys-Leu-Trp-Glu-Leu-Gly-Glu-Asn-Leu-Leu-Glu-Arg-Phe-Leu-Asp-Leu-
Val-Inp
(SEQ ID NO. 106)
Lys-Leu-Lys-Gln-Lys-Leu-Ala-Glu-Leu-Gly-Glu-Asn-Trp-Leu-Glu-Arg-Phe-Leu-Asp-Leu-
Val-Inp
(SEQ ID NO. 107)
Orn-Leu-Orn-Gln-Orn-Leu-Ala-Glu-Leu-Gly-Glu-Asn-Leu-Leu-Glu-Arg-Phe-Leu-Asp-Leu-
Val-Inp
(SEQ ID NO. 108)
Lys-Leu-Lys-Gln-Lys-Leu-Ala-Glu-Leu-Gly-Glu-Asn-Leu-Leu-Glu-Arg-Phe-Phe-Asp-Leu-
Val-Inp
(SEQ ID NO. 109)
Lys-Leu-Lys-Gln-Lys-Leu-Ala-Glu-Leu-Gly-Glu-Asn-Leu-Leu-Glu-Arg-Phe-Leu-Glu-Leu-
Val-Inp
(SEQ ID NO. 110)
Lys-Leu-Lys-Gln-Lys-Leu-Ala-Glu-Leu-Leu-Glu-Asn-Leu-Gly-Glu-Arg-Phe-Leu-Asp-Leu-
Val-Inp
(SEQ ID NO. 111)
Lys-Leu-Lys-Gln-Lys-Leu-Ala-Glu-Leu-Leu-Glu-Asn-Gly-Leu-Glu-Arg-Phe-Leu-Asp-Leu-
Val-Inp
(SEQ ID NO. 112)
Lys-Leu-Lys-Gln-Lys-Leu-Ala-Glu-Gly-Leu-Glu-Asn-Leu-Leu-Glu-Arg-Phe-Leu-Asp-Leu-
Val-Inp
(SEQ ID NO. 113)
Lys-Leu-Lys-Gln-Lys-Leu-Gly-Glu-Leu-Leu-Glu-Asn-Leu-Leu-Glu-Arg-Phe-Leu-Asp-Leu-
Val-Inp
(SEQ ID NO. 114)
Lys-Leu-Lys-Gln-Lys-Gly-Ala-Glu-Leu-Leu-Glu-Asn-Leu-Leu-Glu-Arg-Phe-Leu-Asp-Leu-
Val-Inp
(SEQ ID NO. 115)
Lys-Leu-Lys-Gln-Lys-Leu-Nal-Glu-Leu-Leu-Glu-Asn-Leu-Leu-Glu-Arg-Phe-Leu-Asp-Leu-
Val-Inp
(SEQ ID NO. 116)
Lys-Leu-Lys-Gln-Lys-Leu-Ala-Glu-Leu-Leu-Glu-Asn-Leu-Leu-Glu-Arg-Phe-Leu-Asp-Leu-
Val-Inp
(SEQ ID NO. 117)
Lys-Leu-Lys-Gln-Lys-Leu-Ala-Glu-Leu-Aib-Glu-Asn-Leu-Leu-Glu-Arg-Phe-Leu-Asp-Leu-
Val-Inp
(SEQ ID NO. 118)
Lys-Leu-Lys-Gln-Lys-Leu-Ala-Glu-Leu-Leu-Glu-Asn-Leu-Leu-Glu-Lys-Phe-Leu-Asp-Leu-
Val-Inp
(SEQ ID NO. 119)
Lys-Leu-Lys-Gln-Lys-Nal-Ala-Glu-Leu-Leu-Glu-Asn-Leu-Leu-Glu-Arg-Phe-Leu-Asp-Leu-
Val-Inp
(SEQ ID NO. 120)
Lys-Leu-Lys-Gln-Lys-Leu-Ala-Glu-Leu-Leu-Glu-Gln-Leu-Leu-Glu-Lys-Phe-Leu-Asp-Leu-
Val-Inp
(SEQ ID NO. 121)
Orn-Leu-Orn-Gln-Orn-Leu-Ala-Glu-Leu-Leu-Glu-Asn-Leu-Leu-Glu-Arg-Phe-Leu-Asp-Leu-
Val-Inp
(SEQ ID NO. 122)
Lys-Leu-Lys-Gln-Lys-Leu-Ala-Glu-Leu-Leu-Glu-Asn-Trp-Leu-Glu-Arg-Phe-Leu-Asp-Leu-
Val-Inp
(SEQ ID NO. 123)
Lys-Leu-Lys-Gln-Lys-Leu-Ala-Glu-Leu-Leu-Glu-Asn-Leu-Leu-Glu-Arg-Leu-Leu-Asp-Leu-
Val-Inp
(SEQ ID NO. 124)
Lys-Leu-Lys-Gln-Lys-Leu-Ala-Glu-Leu-Leu-Glu-Gln-Leu-Leu-Glu-Lys-Phe-Leu-Glu-Leu-
Val-Inp
(SEQ ID NO. 125)
Lys-Leu-Lys-Gln-Lys-Leu-Ala-Glu-Leu-Leu-Glu-Gln-Leu-Leu-Glu-Lys-Phe-Leu-Glu-Leu-
Leu-Inp
(SEQ ID NO. 126)
Lys-Leu-Lys-Gln-Lys-Leu-Ala-Glu-Leu-Aib-Glu-Asn-Leu-Leu-Glu-Arg-Phe-Phe-Asp-Leu-
Val-Inp
(SEQ ID NO. 127)
Lys-Leu-Lys-Gln-Lys-Leu-Leu-Glu-Leu-Leu-Glu-Asn-Leu-Leu-Glu-Arg-Phe-Leu-Asp-Leu-
Val-Inp
(SEQ ID NO. 128)
Lys-Leu-Lys-Gln-Lys-Leu-Ala-Glu-Nal-Leu-Glu-Asn-Leu-Leu-Glu-Arg-Phe-Leu-Asp-Leu-
Val-Inp
(SEQ ID NO. 129)
Lys-Leu-Lys-Gln-Lys-Leu-Ala-Glu-Trp-Leu-Glu-Asn-Leu-Leu-Glu-Arg-Phe-Leu-Asp-Leu-
Val-Inp
(SEQ ID NO. 130)
Orn-Leu-Orn-Gln-Orn-Leu-Ala-Glu-Leu-Leu-Glu-Asn-Leu-Leu-Glu-Orn-Phe-Leu-Asp-Leu-
Val-Inp
(SEQ ID NO. 131)
Lys-Leu-Lys-Gln-Lys-Leu-Phe-Glu-Leu-Leu-Glu-Asn-Leu-Leu-Glu-Arg-Phe-Leu-Asp-Leu-
Val-Inp
(SEQ ID NO. 132)
Lys-Leu-Lys-Gln-Arg-Leu-Ala-Asp-Leu-Leu-Glu-Asn-Leu-Leu-Glu-Lys-Phe-Leu-Glu-Leu-
Val-Inp
(SEQ ID NO. 133)
Lys-Leu-Lys-Gln-Lys-Leu-Ala-Glu-Leu-Leu-Glu-Gln-Leu-Leu-Asp-Lys-Phe-Leu-Glu-Leu-
Ala-Inp
(SEQ ID NO. 134)
Lys-Leu-Lys-Gln-Lys-Leu-Trp-Glu-Leu-Leu-Glu-Asn-Leu-Leu-Glu-Arg-Phe-Leu-Asp-Leu-
Val-Nip
(SEQ ID NO. 135)
Lys-Leu-Lys-Gln-Lys-Leu-Ala-Glu-Leu-Gly-Glu-Asn-Leu-Leu-Glu-Arg-Phe-Leu-Asp-Leu-
Val-Nip
(SEQ ID NO. 136)
Lys-Leu-Lys-Gln-Lys-Nal-Ala-Glu-Leu-Gly-Glu-Asn-Leu-Leu-Glu-Arg-Phe-Leu-Asp-Leu-
Val-Nip
(SEQ ID NO. 137)
Lys-Leu-Lys-Gln-Lys-Leu-Trp-Glu-Leu-Gly-Glu-Asn-Leu-Leu-Glu-Arg-Phe-Leu-Asp-Leu-
Val-Nip
(SEQ ID NO. 138)
Lys-Leu-Lys-Gln-Lys-Leu-Ala-Glu-Leu-Gly-Glu-Asn-Trp-Leu-Glu-Arg-Phe-Leu-Asp-Leu-
Val-Nip
(SEQ ID NO. 139)
Orn-Leu-Orn-Gln-Orn-Leu-Ala-Glu-Leu-Gly-Glu-Asn-Leu-Leu-Glu-Arg-Phe-Leu-Asp-Leu-
Val-Nip
(SEQ ID NO. 140)
Lys-Leu-Lys-Gln-Lys-Leu-Ala-Glu-Leu-Gly-Glu-Asn-Leu-Leu-Glu-Arg-Phe-Phe-Asp-Leu-
Val-Nip
(SEQ ID NO. 141)
Lys-Leu-Lys-Gln-Lys-Leu-Ala-Glu-Leu-Gly-Glu-Asn-Leu-Leu-Glu-Arg-Phe-Leu-Glu-Leu-
Val-Nip
(SEQ ID NO. 142)
Lys-Leu-Lys-Gln-Lys-Leu-Ala-Glu-Leu-Leu-Glu-Asn-Leu-Gly-Glu-Arg-Phe-Leu-Asp-Leu-
Val-Nip
(SEQ ID NO. 143)
Lys-Leu-Lys-Gln-Lys-Leu-Ala-Glu-Leu-Leu-Glu-Asn-Gly-Leu-Glu-Arg-Phe-Leu-Asp-Leu-
Val-Nip
(SEQ ID NO. 144)
Lys-Leu-Lys-Gln-Lys-Leu-Ala-Glu-Gly-Leu-Glu-Asn-Leu-Leu-Glu-Arg-Phe-Leu-Asp-Leu-
Val-Nip
(SEQ ID NO. 145)
Lys-Leu-Lys-Gln-Lys-Leu-Gly-Glu-Leu-Leu-Glu-Asn-Leu-Leu-Glu-Arg-Phe-Leu-Asp-Leu-
Val-Nip
(SEQ ID NO. 146)
Lys-Leu-Lys-Gln-Lys-Gly-Ala-Glu-Leu-Leu-Glu-Asn-Leu-Leu-Glu-Arg-Phe-Leu-Asp-Leu-
Val-Nip
(SEQ ID NO. 147)
Lys-Leu-Lys-Gln-Lys-Leu-Nal-Glu-Leu-Leu-Glu-Asn-Leu-Leu-Glu-Arg-Phe-Leu-Asp-Leu-
Val-Nip
(SEQ ID NO. 148)
Lys-Leu-Lys-Gln-Lys-Leu-Ala-Glu-Leu-Leu-Glu-Asn-Leu-Leu-Glu-Arg-Phe-Leu-Asp-Leu-
Val-Nip
(SEQ ID NO. 149)
Lys-Leu-Lys-Gln-Lys-Leu-Ala-Glu-Leu-Aib-Glu-Asn-Leu-Leu-Glu-Arg-Phe-Leu-Asp-Leu-
Val-Nip
(SEQ ID NO. 150)
Lys-Leu-Lys-Gln-Lys-Leu-Ala-Glu-Leu-Leu-Glu-Asn-Leu-Leu-Glu-Lys-Phe-Leu-Asp-Leu-
Val-Nip
(SEQ ID NO. 151)
Lys-Leu-Lys-Gln-Lys-Nal-Ala-Glu-Leu-Leu-Glu-Asn-Leu-Leu-Glu-Arg-Phe-Leu-Asp-Leu-
Val-Nip
(SEQ ID NO. 152)
Lys-Leu-Lys-Gln-Lys-Leu-Ala-Glu-Leu-Leu-Glu-Gln-Leu-Leu-Glu-Lys-Phe-Leu-Asp-Leu-
Val-Nip
(SEQ ID NO. 153)
Orn-Leu-Orn-Gln-Orn-Leu-Ala-Glu-Leu-Leu-Glu-Asn-Leu-Leu-Glu-Arg-Phe-Leu-Asp-Leu-
Val-Nip
(SEQ ID NO. 154)
Lys-Leu-Lys-Gln-Lys-Leu-Ala-Glu-Leu-Leu-Glu-Asn-Trp-Leu-Glu-Arg-Phe-Leu-Asp-Leu-
Val-Nip
(SEQ ID NO. 155)
Lys-Leu-Lys-Gln-Lys-Leu-Ala-Glu-Leu-Leu-Glu-Asn-Leu-Leu-Glu-Arg-Leu-Leu-Asp-Leu-
Val-Nip
(SEQ ID NO. 156)
Lys-Leu-Lys-Gln-Lys-Leu-Ala-Glu-Leu-Leu-Glu-Gln-Leu-Leu-Glu-Lys-Phe-Leu-Glu-Leu-
Val-Nip
(SEQ ID NO. 157)
Lys-Leu-Lys-Gln-Lys-Leu-Ala-Glu-Leu-Leu-Glu-Gln-Leu-Leu-Glu-Lys-Phe-Leu-Glu-Leu-
Leu-Nip
(SEQ ID NO. 158)
Lys-Leu-Lys-Gln-Lys-Leu-Ala-Glu-Leu-Aib-Glu-Asn-Leu-Leu-Glu-Arg-Phe-Phe-Asp-Leu-
Val-Nip
(SEQ ID NO. 159)
Lys-Leu-Lys-Gln-Lys-Leu-Leu-Glu-Leu-Leu-Glu-Asn-Leu-Leu-Glu-Arg-Phe-Leu-Asp-Leu-
Val-Nip
(SEQ ID NO. 160)
Lys-Leu-Lys-Gln-Lys-Leu-Ala-Glu-Nal-Leu-Glu-Asn-Leu-Leu-Glu-Arg-Phe-Leu-Asp-Leu-
Val-Nip
(SEQ ID NO. 161)
Lys-Leu-Lys-Gln-Lys-Leu-Ala-Glu-Trp-Leu-Glu-Asn-Leu-Leu-Glu-Arg-Phe-Leu-Asp-Leu-
Val-Nip
(SEQ ID NO. 162)
Orn-Leu-Orn-Gln-Orn-Leu-Ala-Glu-Leu-Leu-Glu-Asn-Leu-Leu-Glu-Orn-Phe-Leu-Asp-Leu-
Val-Nip
(SEQ ID NO. 163)
Lys-Leu-Lys-Gln-Lys-Leu-Phe-Glu-Leu-Leu-Glu-Asn-Leu-Leu-Glu-Arg-Phe-Leu-Asp-Leu-
Val-Nip
(SEQ ID NO. 164)
Lys-Leu-Lys-Gln-Arg-Leu-Ala-Asp-Leu-Leu-Glu-Asn-Leu-Leu-Glu-Lys-Phe-Leu-Glu-Leu-
Val-Nip
(SEQ ID NO. 165)
Lys-Leu-Lys-Gln-Lys-Leu-Ala-Glu-Leu-Leu-Glu-Gln-Leu-Leu-Asp-Lys-Phe-Leu-Glu-Leu-
Ala-Nip

Other Apo A-I analogues for use in the present invention include a 23 to 29 residue peptide comprising any one of SEQ ID NO. 102-SEQ ID NO. 165.

Apolipoprotein complexes, comprising the Apo A-I analogues according to Formula 2 and described herein, may contain multiple types of phospholipids in the lipid fraction of the apolipoprotein complex including but not limited to one of more phospholipids selected from, sphingomyelin (SPH), 1,2-dipalmitoyl-sn-glycero-3-phosphocholine (DPPC) and 1,2-dipalmitoyl-sn-glycero-3-[phospho-rac-(1-glycerol)] (DPPG). Preferably the lipid composition of the apolipoprotein complex is 48.5% SPH/48.5% DPPC/3% DPPG (w/w/w).

Apolipoprotein complexes, comprising the Apo A-I analogues according to Formula 2 and described herein, may contain essentially sphingomyelin in the lipid fraction in combination with about 3% wt/wt of a negatively charged phospholipid selected from phosphatidylinositol, phosphatidylserine, phosphatidylglycerol, phosphatidic acid, and mixtures thereof. Either D-erythrose-sphingomyelin and/or D-erythrose dihydrosphingomyelin or any combination thereof can be used as the neutral amino acid. The acyl chains of the sphingomyelin or other negatively charged phospholipids in the lipid phase are selected from a saturated, a mono-unsaturated and a polyunsaturated hydrocarbon containing from 6 to 24 carbon atoms and may differ in the degree of saturation.

Apolipoprotein complexes for use in the invention, comprising the Apo A-I analogues described above ([00117] to [00143]) containing a ratio of peptide to phospholipid between 1:2 and 1:20. The ratio of peptide to phospholipid can be 1:2, 1:5, 1:6, 1:7, 1:8, 1:9, 1:10, 1:11, 1:12, 1:13, 1:14, 1:15, 1:16, 1:17, 1:18, 1:19, 1:20 or any ratio in between. Some apolipoprotein complexes, for use in the present invention, comprising an Apo A-I analogue according to Formula 2 and described herein, have a ratio peptide to phospholipid that is between 1:2 and 1:3 and preferably 1:2.5.

The apolipoprotein complexes for use in the present invention, to treat LVDD, can be administered by any suitable route that ensures bioavailability in the circulation. This may be achieved by parenteral routes of administration, including intravenous (IV), intramuscular (IM), intradermal, subcutaneous (SC) and intraperitoneal (IP) injections. However, other routes of administration can be used. For example, absorption through the gastrointestinal tract may be accomplished by oral routes of administration (including but not limited to ingestion, buccal and sublingual routes) provided appropriate formulations (e.g., enteric coatings) are used to avoid or minimize degradation of the peptides, e.g., in the harsh environments of the oral mucosa, stomach and/or small intestine. Alternatively, administration via mucosal tissue such as vaginal and rectal modes of administration may be utilized to avoid or minimize degradation in the gastrointestinal tract. In yet another alternative, the apolipoprotein complex may be administered transcutaneously (e.g., transdermally), ocularly, or by inhalation. It will be appreciated that the route of administration chosen may vary with the condition, age and compliance of the recipient.

The actual dose of the apolipoprotein complex used can vary with the route of administration, and can be adjusted to achieve circulating plasma concentrations of apolipoprotein complex of 100 mg/L to 2 g/L. In one embodiment, the dose of apolipoprotein complex is adjusted to achieve a serum level of apolipoprotein complex for at least 24 hours following administration that is in the range of about 10 mg/dL to 300 mg/dL higher than a baseline (initial) level prior to administration.

Apolipoprotein complexes may be administered in a variety of different treatment regimens. In one embodiment, the apolipoprotein complex is administered by injection at a dose between 0.5 mg/kg to 100 mg/kg once a week. In another embodiment, desirable serum levels may be maintained by continuous infusion or by intermittent infusion providing about 0.5 mg/kg/hr to 100 mg/kg/hr of the apolipoprotein complex. In one embodiment, the apolipoprotein complex is administered at a dose of about 20 mg/kg.

In another embodiment, the apolipoprotein complex is administered by intravenous injection once or more per day. In another embodiment, the apolipoprotein complex is administered by injection once every 3 to 15 days, once every 5 to 10 days, or once every 10 days. In another embodiment, the apolipoprotein complex is administered in a series of maintenance injections, where the series of maintenance injections is administered once every 6 months to one year. The series of maintenance injections can be administered, for example, over one day (perfusion to maintain a specified plasma level of complexes), several days (e.g., four injections over a period of eight days) or several weeks (e.g., four injections over a period of four weeks). In particular embodiments, the mode of administration is intravenously and the dosage is from about 1 mg/kg to about 100 mg/kg or sometimes even higher (e.g., from about 1 mg/kg to about 150 mg/kg, from about 1 mg/kg to about 175 mg/kg, from about 1 mg/kg to about 200 mg/kg, from about 1 mg/kg to about 250 mg/kg, from about 1 mg/kg to about 275 mg/kg, or from about 1 mg/kg to about 300 mg/kg). In certain embodiments, the frequency of injections is from daily to weekly and for a period of from one or more days (e.g., one, two, three, four, five, six, or seven day(s)) to one or more months (e.g., one, two, three, four, five, or six month(s)).

### III. Small Molecule Compounds and Pharmaceutical Compositions Thereof Useful for Treating Left Ventricular Diastolic Dysfunction (LVDD)

Other small molecule compounds, known in the art, which can be used to reduce cholesterol and other lipids from extrahepatic tissues to a degree similar to that of a pharmaceutical composition comprising an apolipoprotein complex, are also useful in the present invention. Nonlimiting examples of such compounds for use in the present invention include cholesterol ester transfer protein (CETP) inhibitors, ABCA1 agonists, and anti-microRNA-33 (anti-miR-33) compounds, such as miR-33 antagomirs.

miR-33 antagomirs include small synthetic RNAs that are sufficiently complementary (e.g., up to 85%, 90%, 95%, or even 100% complementary) to a miR-33 sequence, such that the synthetic RNA exerts a gene silencing effect by hybridizing the miR-33 sequence. Exemplary miR-33 sequences include but are not limited to those listed below:

SEQ ID NO. 166: human mir-33a (hsa-mir-33a
MI0000091), stem loop
CUGUGGUGCAUUGUAGUUGCAUUGCAUGUUCUGGUGGUACCCAUGCAAU
GUUUCCACAGUGCAUCACAG
SEQ ID NO. 167: human mir-33a (hsa-miR-33a
MIMAT0000091), mature sequence
GUGCAUUGUAGUUGCAUUGCA
SEQ ID NO. 168: human mir-33a (hsa-miR-33a*
MIMAT0004506), minor sequence
CAAUGUUUCCACAGUGCAUCAC
SEQ ID NO. 169: human mir-33b (hsa-mir-33b
MI0003646), stem loop
GCGGGCGGCCCCGCGGUGCAUUGCUGUUGCAUUGCACGUGUGUGAGGCG
GGUGCAGUGCCUCGGCAGUGCAGCCCGGAGCCGGCCCCUGGCACCAC
SEQ ID NO. 170: human mir-33b (hsa-miR-33b
MIMAT0003301), mature sequence
GUGCAUUGCUGUUGCAUUGC
SEQ ID NO. 171: human mir-33b (hsa-miR-33b*
MIMAT0004811), minor sequence
CAGUGCCUCGGCAGUGCAGCCC
SEQ ID NO. 172: mouse mir-33 (mmu-mir-33
MI0000707), stem loop
CUGUGGUGCAUUGUAGUUGCAUUGCAUGUUCUGGCAAUACCUGUGCAAU
GUUUCCACAGUGCAUCACGG
SEQ ID NO. 173: mouse mir-33 (mmu-miR-33
MIMAT0000667), mature sequence
GUGCAUUGUAGUUGCAUUGCA
SEQ ID NO. 174: mouse mir-33 (mmu-miR-33*
MIMAT0004666), minor sequence
CAAUGUUUCCACAGUGCAUCAC

Accordingly, exemplary miR-33 antagomirs include synthetic RNAs that are sufficiently complementary (e.g., up to 85%, 90%, 95%, or even 100% complementary) to any one of SEQ ID NO. 166-174, or fragments thereof, as well as any other useful anti-miRNA-33 compounds known in the art (see, e.g., Najafi-Shoushtari et al., “MicroRNA-33 and the SREBP host genes cooperate to control cholesterol homeostasis.” Science 2010 Jun. 18; 328(5985):1566-1569, epub 2010 May 13; Rayner et al., “MiR-33 contributes to the regulation of cholesterol homeostasis,” Science 2010 Jun. 18; 328(5985):1570-1573, epub 2010 May 13; Marquart et al., “miR-33 links SREBP-2 induction to repression of sterol transporters,” Proc Natl Acad Sci USA 2010 Jul. 6; 107(27):12228-12232, epub 2010 Jun. 21).

The present invention includes the use of a cholesterol ester transfer protein (CETP) inhibitor for the treatment of left ventricular diastolic dysfunction. In one embodiment, the CETP inhibitor has a bis-(2-aminophenyl) disulfide structure or a 2-amino-phenylthio structure. In a preferred embodiment, the CETP inhibitor is Dalcetrapib (Propanethioic acid, 2-methyl-, S-[2-[[[1-(2-ethylbutyl)cyclohexyl]carbonyl]amino]phenyl] ester) according to Formula 3.

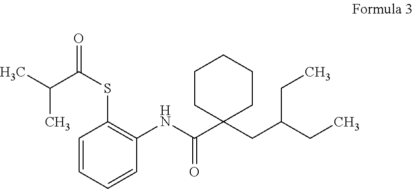 [SMILES: CCC(CC)CC1(C(=O)Nc2ccccc2SC(=O)C(C)C)CCCCC1]

Other CETP inhibitors for use in the present invention include compounds similar to Dalceptrpib as described in U.S. Pat. No. 6,753,346, which is hereby incorporated by reference.

Non limiting examples of a CETP inhibitor for use in the present invention include a compound selected from the group consisting of:
S-[2-(1-isopentylcyclohexanecarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentyleyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxy-thioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetyl amino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)-phenyl]2,2-dimethylthiopropionate;
S-[4,5-dichloro-2-(1-isopentylcyclopentanecarbonylamino)-phenyl]2,2-dimethylthiopropionate;
S-[2-(1-isopentylcyclohexanecarbonylamino)-4-trifluoromethylphenyl]2,2-dimethylthiopropionate;
O-methyl S-[2-(1-isopentylcyclohexanecarbonylaminophenyl monothiocarbonate;
S-[2-(1-methylcyclohexanecarbonylamino)phenyl]S-phenyldithiocarbonate;
S-[2-(1-isopentylcyclohexanecarbonylamino)phenyl]N-phenylthiocarbamate;
S-[2-(pivaloylamino)-4-trifluoromethylphenyl]2,2-dimethylthiopropionate;
S-[4,5-dichloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate;
S-[4,5-dichloro-2-(1-pentylcyclohexanecarbonylamino)-phenyl]2,2-dimethylthiopropionate;
S-[4,5-dichloro-2-(1-cyclopropylmethylcyclohexanecarbonylamino)phenyl]2,2-dimethylthiopropionate;
S-[4,5-dichloro-2-(1-cyclohexylmethylcyclohexanecarbonylamino)phenyl]2,2-dimethylthiopropionate;
S[4,5-dichloro-2-(1-isopropylcyclohexanecarbonylamino)-phenyl]2,2-dimethylthiopropionate;
S-[4,5-dichloro-2-(1-isopentylcycloheptanecarbonylamino)-phenyl]2,2-dimethylthiopropionate;
S-[4,5-dichloro-2-(1-isopentylcyclobutanecarbonylamino)-phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethylthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)-phenyl]2,2-dimethylthiopropionate;
S-[5-fluoro-2-(1-isopentylcyclohexanecarbonylamino)phenyl]2,2-dimethylthiopropionate;
bis-[4,5-dichloro-2-(1-isopentylcyclohexanecarbonylamino)-phenyl]disulfide;
2-tetrahydrofurylmethyl 2-(1-isopentylcyclohexanecarbonylamino) phenyl disulfide;
N-(2-mercaptophenyl)-1-ethylcyclohexanecarboxamide;
N-(2-mercaptophenyl)-1-propylcyclohexanecarboxamide;
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]thioacetate;
bis-[2-[1-(2-ethylbutyl)cyclohexanecarbonylamino]phenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methylthiopropionate;
S-[2-(1-isobutylcyclohexanecarbonylamino)phenyl]2-methylthiopropionate;
S-[2-[1-(2-ethylbutyl)cyclohexanecarbonylamino]phenyl]1-acetylpiperidine-4-thiocarboxylate;
S-[2-[1-(2-ethylbutyl)cyclohexanecarbonylamino]phenyl]thioacetate;
S-[2-[1-(2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-[1-(2-ethylbutyl)cyclohexanecarbonylamino]phenyl]2-hydroxy-2-methylthiopropionate;
S-[2-[1-(2-ethylbutyl)cyclohexanecarbonylamino]phenyl]4-chlorophenoxythioacetate;
S-[2-(1-isobutylcyclohexanecarbonylamino)phenyl]4-chlorophenoxythioacetate; and
S-[2-(1-isobutylcyclohexanecarbonylamino)phenyl]-1-acetyl-piperidine-4-thiocarboxylate,
   or a pro-drug compound, a pharmaceutically acceptable salt, hydrate, or solvate thereof

Other CETP inhibitors for use in the present invention include Anacetrapib ((4S,5R)-5-[3,5-bis(trifluoromethyl)phenyl]-3-{[4′-fluoro-2′-methoxy-5′-(propan-2-yl)-4-(trifluoromethyl)[1,1′-biphenyl]-2-yl]methyl}-4-methyl-1,3-oxazolidin-2-one) and similar compounds. Anacetrapib is represented by formula 4 below

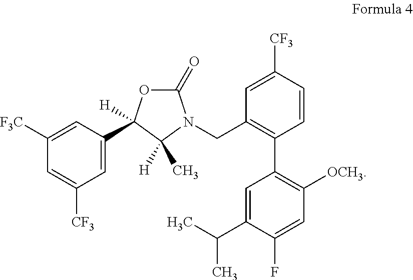 [SMILES: COc1cc(F)c(C(C)C)cc1-c1ccc(C(F)(F)F)cc1CN1C(=O)O[C@H](c2cc(C(F)(F)F)cc(C(F)(F)F)c2)[C@@H]1C]

Other CETP inhibitors for use in the present invention include compounds disclosed in U.S. Pat. No. 7,652,049, hereby incorporated by reference. For example, compounds according to Formula 5 below, wherein:

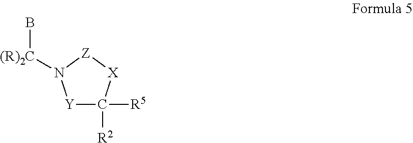

### Y is —(CRR¹)— or —C(═O)—

X is selected from the group consisting of —O—, —NH—, —N(C₁-C₅ alkyl)-, and (CRR⁶)—
Z is selected from the group consisting of —C(═O)—, —S(O)₂—, and —C(═N—R⁹)—, wherein R⁹ is selected from the group consisting of H, —CN, and CH₃;
each R is independently selected from the group consisting of H and C₁-C₅alkyl and halogen, wherein C₁-C₅ alkyl is optionally substituted with 1-11 halogens;
B is selected from the group consisting of A¹ and A², wherein A¹ has the structure:

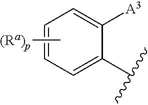

R¹ and R⁶ are each selected from the group consisting of H, —C₁-C₅ alkyl, halogen, and —(C(R)₂)ₙA², wherein —C₁-C₅alkyl is optionally substituted with 1-11 halogens;
R² is selected from the group consisting of H, —C₁-C₅ alkyl, and —(C(R)₂)ₙA², wherein —C₁-C₅ alkyl is optionally substituted with 1-11 halogens;
wherein one of B and R² is A¹; and one of B, R¹, and R² is A² or —(C(R)₂)ₙA²; so that the compound of Formula I comprises one group A′ and one group A²;
A³ is selected from the group consisting of: (a) an aromatic ring selected from phenyl and napthyl; (b) a 5-6-membered non-aromatic cycloalkyl ring, which optionally comprises 1-2 heteroatoms independently selected from N, S, O, and —N(O)—, wherein the point of attachment of A³ to the phenyl ring to which A³ is attached is a carbon atom; and (c) a benzoheterocyclic ring comprising a phenyl ring fused to a 5-membered aromatic heterocyclic ring having 1-2 heteroatoms independently selected from 0, N, and —S(O)ₓ, wherein the point of attachment of A₃ to the phenyl ring to which A³ is attached is a carbon atom;
A² is selected from the group consisting of: (a) an aromatic ring selected from phenyl and napthyl; (b) a benzoheterocyclic ring comprising a phenyl ring fused to a 5-membered aromatic heterocyclic ring having 1-2 heteroatoms independently selected from 0, N, and —S; (c) a 5-6-membered heterocyclic ring having 1-4 heteroatoms independently selected from N, S, O, and —N(O)—, and optionally also comprising 1-3 double bonds; (d) a benzoheterocyclic ring comprising a phenyl ring fused to a 5-membered heterocyclic ring having 1-2 heteroatoms independently selected from O, N, and S; and (e) a —C₅-C₆ cycloalkyl ring optionally having 1-3 double bonds;
each R^{a} is independently selected from the group consisting of —C₁-C₆ alkyl, —C₂-C₆ alkenyl, —C₂-C₆ alkynyl, —C₃-C₈ cycloalkyl optionally having 1-3 double bonds, —OC₁-C₆ alkyl, —OC₂-C₆ alkenyl, —OC₂-C₆ alkynyl, —OC₃-C₈ cycloalkyl optionally having 1-3 double bonds, —C(═O)C₁-C₆ alkyl, —C(═O)C₃-C₈ cycloalkyl, —C(═O)OH, —CO₂H, —CO₂C₁-C₆-alkyl, —C(═O)SC₁-C₆ alkyl, —OH, —NR³R⁴, —C(═O)NR³R⁴, —NR³C(═O)OC₁-C₆ alkyl, —NR³C(═O)NR³R⁴, —S(O)—C₁-C₆ alkyl, —S(O)_{y}NR³R⁴, —NR³S(O)_{y}NR³R⁴, halogen, —CN, —NO₂, and a 5-6-membered heterocyclic ring having 1-4 heteroatoms independently selected from N, S, and O, said heterocyclic ring optionally also comprising a carbonyl group and optionally also comprising 1-3 double bonds, wherein the point of attachment of said heterocyclic ring to the ring to which R^{a} is attached is a carbon atom, wherein said heterocyclic ring is optionally substituted with 1-5 substituent groups independently selected from halogen, —C₁-C₃ alkyl, and —OC₁-C₃ alkyl, wherein —C₁-C₃ alkyl and —OC₁-C₃ alkyl are optionally substituted with 1-7 halogens; wherein for compounds in which R^{a} is selected from the group consisting of —C₁-C₆ alkyl, —C₂-C₆ alkenyl, —C₂-C₆ alkynyl, —C₃-C₈ cycloalkyl optionally having 1-3 double bonds, —OC₁-C₆ alkyl, —OC₂-C₆ alkenyl, —OC₂-C₆ alkynyl, —OC₃-C₈ cycloalkyl optionally having 1-3 double bonds, —C(═O)C₁-C₆ alkyl, —C(═O)C₃-C₈ cycloalkyl, —CO₂C₁-C₆ alkyl, —C(═O)SC₁-C₆ alkyl, —NR₃C(═O)OC₁-C₆ alkyl, and —S(O)ₓC₁-C₆ alkyl, R^{a} is optionally substituted with 1-15 halogens and is optionally also substituted with 1-3 substituent groups independently selected from (a) —OH, (b) —CN, (c) —NR³R⁴, (d) —C₃-C₈ cycloalkyl optionally having 1-3 double bonds and optionally substituted with 1-15 halogens, (e) —OC₁-C₄ alkyl optionally substituted with 1-9 halogens and optionally also substituted with 1-2 substituent groups independently selected from —OC₁-C₂ alkyl and phenyl, (f) —OC₃-C₈ cycloalkyl optionally having 1-3 double bonds and optionally substituted with 1-15 halogens, (g) —CO₂H, (h) —C(═O)CH₃, (i) —CO₂C₁-C₄ alkyl which is optionally substituted with 1-9 halogens, and (j) phenyl which is optionally substituted with 1-3 groups independently selected from halogen, —CH₃, —CF₃, —OCH₃, and —OCF₃; with the proviso that when B is A¹, and X and Y are —CH₂—, and Z is —C(═O)—, and R² is phenyl which has a substituent R^{a} in the 4-position, wherein R^{a} is —OC₁-C₆ alkyl which is optionally substituted as described above, then there are no other R^{a} substitutents on R² in which R^{a} is selected from —OH, —OC₁-C₆ alkyl, —OC₂-C₆ alkenyl, —OC₂-C₆ alkynyl, and —OC₃-C₆ cycloalkyl optionally having 1-3 double bonds, optionally substituted as described above;
n is 0 or 1;
p is an integer from 0-4;
x is 0, 1, or 2;
y is 1 or 2;
R³ and R⁴ are each independently selected from H, —C₁-C₅ alkyl, —C(═O)C₁-C₅ alkyl and —S(O)_{y}C₁-C₅ alkyl, wherein —C₁-C₅ alkyl in all instances is optionally substituted with 1-11 halogens; and
R⁵ is selected from the group consisting of H, —OH, —C₁-C₅alkyl, and halogen, wherein —C₁-C₅ alkyl is optionally substituted with 1-11 halogens.

Doses of CETP inhibitors are preferably orally administered at a dose for an adult of between 1-1000 mg per day or particularly 50-800 mg per day.

In another embodiment, the compounds disclosed in US application number US20080085906A1 (compounds of Formula 6) are of use in the present invention. US application number US20080085906A1 is hereby incorporated by reference.

Additional compounds for use in the present invention include a compound of Formula 6, wherein:

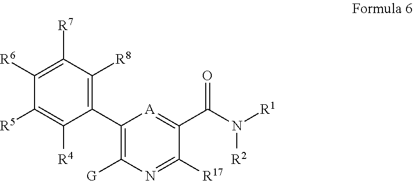

### A is CH;

R² is hydrogen and R¹ is selected from the group consisting of: (a) cycloalkyl, which is optionally substituted by hydroxy, lower hydroxyalkyl or lower alkoxy, (b) 1-hydroxy-2-indanyl, (c) lower hydroxyalkyl, (d) lower hydroxyhalogenalkyl, (e) lower hydroxyalkoxyalkyl, (f) —CH₂—CR⁹R¹⁰-cycloalkyl, wherein R⁹ is hydrogen or lower alkyl; and wherein R¹⁰ is hydrogen, hydroxy or lower alkoxy; and (g) —CR¹¹R¹²—COOR¹³; wherein R¹¹ and R¹² independently from each other are hydrogen or lower alkyl; and wherein R¹³ is lower alkyl; or alternatively, R¹ and R² together with the nitrogen atom to which they are attached form a morpholinyl ring;
G is a group selected from the group consisting of: (a) —X—R³, wherein X is O or NR¹⁴, wherein R¹⁴ is selected from the group consisting of hydrogen, lower alkyl and lower hydroxyalkyl; and R³ is lower cycloalkylalkyl, (b) —C═C—R¹⁵, wherein R¹⁵ is selected from the group consisting of lower alkoxyalkyl, cycloalkyl and furanyl substituted by halogen; and (c) —CH₂—CH₂—R¹⁶,
wherein R¹⁶ is selected from the group consisting of: (1) a cycloalkyl which is optionally substituted by hydroxy or lower alkoxy, (2) a heteroaryl which is pyridyl or imidazolyl, which is optionally substituted by lower alkyl or halogen, and (3) lower alkylaminocarbonyl or lower alkylcarbonylamino;
R⁴ and R⁸ independently from each other are hydrogen or halogen;
R⁵ and R⁷ independently from each other are selected from the group consisting of hydrogen, lower alkyl, lower alkoxy, halogen, lower halogenalkyl, lower halogenalkoxy and cyano;
R⁶ is selected from the group consisting of hydrogen, lower alkoxy, halogen, lower halogenalkyl, lower halogenalkoxy and cyano; and
R¹⁷ is a lower halogenalkyl.

Compounds of the genus described herein according to Formula 6, are preferably those where (a) X is 0, R¹ is —CH₂—CR⁹R¹⁰-cycloalkyl, R⁹ is hydrogen and R¹⁰ is hydroxyl; (b) R⁶ is halogen or lower halogenalkyl and R⁴, R⁵, R⁷ and R⁸ are hydrogen; (c) R′ is cycloalkyl which is substituted by hydroxy, or —CH₂—CR⁹R¹⁰-cycloalkyl, R⁹ is hydrogen or lower alkyl, R¹⁰ is hydrogen, hydroxy or lower alkoxy, R² is hydrogen, X is O; R⁴, R⁵, R⁷ and R⁸ are hydrogen, and R⁶ is halogen.

In particular, the compound 5-(4-chloro-phenyl)-6-cyclopropylmethoxy-N-((1R,2R)-2-hydroxy-cyclohexyl)-2-trifluoromethyl-nicotinamide, or a pharmaceutically acceptable salt thereof, which are compounds according to formula 5, are also of use in the present invention.

Other compounds useful in the present invention include those described in US application number US2009/0247588, which is hereby incorporated by reference. US application number US2009/0247588 discloses a family of compounds according to Formula 7, wherein:

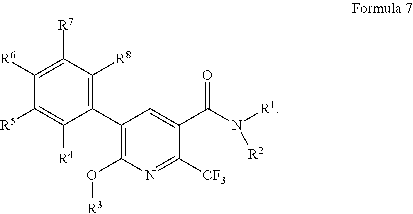

R¹ is selected from the group consisting of: (1) lower hydroxyalkyl, (2) cycloalkyl which is unsubstituted or substituted by hydroxy or lower hydroxyalkyl, and (3) —CH₂—CR⁹R¹⁰-cycloalkyl, wherein R⁹ is hydrogen or lower alkyl, and R¹⁰ is hydrogen or hydroxy;
R² is hydrogen;
R³ is selected from the group consisting of: (1) lower alkoxyalkyl, (2) lower halogenalkyl, and (3) lower heteroarylalkyl, wherein the heteroaryl group is unsubstituted or substituted once or twice by lower alkyl;
R⁴ and R⁸ are hydrogen; and
R⁵, R⁶ and R⁷ independently from each other are selected from the group consisting of: (1) hydrogen, (2) lower alkyl, (3) halogen, (4) lower halogenalkyl, (5) lower halogenalkoxy, (6) lower alkylsulfonylamino, and (7) cyano.

Additional compounds for use in the present invention, according to Formula 7, include a compound selected from the group consisting of:
5-(4-chloro-phenyl)-N-(2-cyclopropyl-2-hydroxy-propyl)-6-(2,2,2-trifluoro-ethoxy)-2-trifluoromethyl-nicotinamide,
N-(2-cyclopropyl-2-hydroxy-propyl)-5-(3,4-dichloro-phenyl)-6-(2,2,2-trifluoro-ethoxy)-2-trifluoromethyl-nicotinamide,
N—((R)-2-cyclopropyl-2-hydroxy-propyl)-5-(3,4-dichloro-phenyl)-6-(2,2,2-trifluoroethoxy)-2-trifluoromethyl-nicotinamide
N—((S)-2-cyclopropyl-2-hydroxy-propyl)-5-(3,4-dichloro-phenyl)-6-(2,2,2-trifluoroethoxy)-2-trifluoromethyl-nicotinamide
5-(3-chloro-phenyl)-N-(2-cyclopropyl-2-hydroxy-propyl)-6-(2,2,2-trifluoro-ethoxy)-2-trifluoromethyl-nicotinamide,
5-(4-chloro-phenyl)-N-((1R,2R)-2-hydroxy-cyclohexyl)-6-(2,2,2-trifluoro-ethoxy)-2-trifluoromethyl-nicotinamide,
5-(4-chloro-phenyl)-N-((1R,2S)-2-hydroxy-cyclohexyl)-6-(2,2,2-trifluoro-ethoxy)-2-trifluoromethyl-nicotinamide,
5-(4-chloro-phenyl)-N-((1S,2R)-2-hydroxy-cyclohexyl)-6-(2,2,2-trifluoro-ethoxy)-2-trifluoromethyl-nicotinamide,
5-(4-chloro-phenyl)-N-((1S,2S)-2-hydroxy-cyclohexyl)-6-(2,2,2-trifluoro-ethoxy)-2-trifluoromethyl-nicotinamide,
N-(1-hydroxymethyl-3-methyl-butyl)-5-(4-methanesulfonylamino-phenyl)-6-(pyridin-2-ylmethoxy)-2-trifluoromethyl-nicotinamide,
N-((1R,2R)-2-hydroxy-cyclohexyl)-5-(4-methanesulfonylamino-phenyl)-6-(2,2,2-trifluoro-ethoxy)-2-trifluoromethyl-nicotinamide,
5-(3,4-dichloro-phenyl)-N-((1R,2R)-2-hydroxy-cyclohexyl)-6-(pyridin-2-ylmethoxy)-2-trifluoromethyl-nicotinamide, or a pharmaceutically acceptable salt or solvate thereof.

Compounds for use in the present invention also include those described in US application number 2009/0247550, which is hereby incorporated by reference. US application number 2009/0247550 discloses a family of compounds according to Formula 8, wherein:

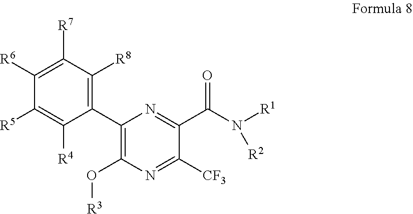

R¹ is selected from the group consisting of: (1) cycloalkyl, which is unsubstituted or substituted by hydroxy or lower hydroxyalkyl, and (2) —CH₂—CR⁹R¹⁰-cycloalkyl, wherein R⁹ is hydrogen or lower alkyl, and R¹⁰ is hydrogen or hydroxy;
R² is hydrogen;
R³ is selected from the group consisting of: (1) lower cycloalkylalkyl, (2) lower alkoxyalkyl, (3) lower halogenalkyl, (4) lower heteroarylalkyl, wherein the heteroaryl group is unsubstituted or substituted once or twice by lower alkyl, and (5) phenyl, which is unsubstituted or substituted once or twice by halogen;
R⁴ and R⁸ independently from each other are hydrogen or halogen; and
R⁵, R⁶ and R⁷ independently from each other are selected from the group consisting of: (1) hydrogen, (2) lower alkyl, (3) lower alkoxy, (4) halogen, (5) lower halogenalkyl, (6) lower halogenalkoxy, (7) lower alkylsulfonylamino, and (8) cyano.

Other compounds for use in the present invention, according to Formula 8, can be selected from the group consisting of:
6-(4-chloro-phenyl)-5-(2,2,2-trifluoro-ethoxy)-3-trifluoromethyl-pyrazine-2-carboxylic acid (2-cyclopropyl-2-hydroxy-propyl)-amide,
6-(4-chloro-phenyl)-5-cyclopropylmethoxy-3-trifluoromethyl-pyrazine-2-carboxylic acid ((1R,2R)-2-hydroxy-cyclohexyl)-amide,
6-(4-chloro-phenyl)-5-cyclopropylmethoxy-3-trifluoromethyl-pyrazine-2-carboxylic acid (2-cyclopropyl-2-hydroxy-propyl)-amide,
6-(3-chloro-phenyl)-5-(2,2,2-trifluoro-ethoxy)-3-trifluoromethyl-pyrazine-2-carboxylic acid ((1R,2R)-2-hydroxy-cyclohexyl)-amide,
6-(3-chloro-phenyl)-5-(2,2,2-trifluoro-ethoxy)-3-trifluoromethyl-pyrazine-2-carboxylic acid (2-cyclopropyl-2-hydroxy-propyl)-amide,
6-(3-chloro-phenyl)-5-cyclopropylmethoxy-3-trifluoromethyl-pyrazine-2-carboxylic acid ((1R,2R)-2-hydroxy-cyclohexyl)-amide,
6-(3-chloro-phenyl)-5-cyclopropylmethoxy-3-trifluoromethyl-pyrazine-2-carboxylic acid (2-cyclopropyl-2-hydroxy-propyl)-amide,
6-(3-chloro-phenyl)-5-phenoxy-3-trifluoromethyl-pyrazine-2-carboxylic acid ((1R,2R)-2-hydroxy-cyclohexyl)-amide,
6-(3-chloro-phenyl)-5-phenoxy-3-trifluoromethyl-pyrazine-2-carboxylic acid (2-cyclopropyl-2-hydroxy-propyl)-amide,
6-(3-chloro-phenyl)-5-(pyridin-2-ylmethoxy)-3-trifluoromethyl-pyrazine-2-carboxylic acid ((1R,2R)-2-hydroxy-cyclohexyl)-amide,
6-(3-chloro-phenyl)-5-(pyridin-2-ylmethoxy)-3-trifluoromethyl-pyrazine-2-carboxylic acid (2-cyclopropyl-2-hydroxy-propyl)-amide,
6-(3,4-dichloro-phenyl)-5-(2,2,2-trifluoro-ethoxy)-3-trifluoromethyl-pyrazine-2-carboxylic acid ((1R,2R)-2-hydroxy-cyclohexyl)-amide,
6-(3,4-dichloro-phenyl)-5-(2,2,2-trifluoro-ethoxy)-3-trifluoromethyl-pyrazine-2-carboxylic acid (2-cyclopropyl-2-hydroxy-propyl)-amide,
6-(3,4-dichloro-phenyl)-5-cyclopropylmethoxy-3-trifluoromethyl-pyrazine-2-carboxylic acid ((1R,2R)-2-hydroxy-cyclohexyl)-amide,
6-(3,4-dichloro-phenyl)-5-cyclopropylmethoxy-3-trifluoromethyl-pyrazine-2-carboxylic acid (2-cyclopropyl-2-hydroxy-propyl)-amide,
6-(3,4-dichloro-phenyl)-5-phenoxy-3-trifluoromethyl-pyrazine-2-carboxylic acid ((1R,2R)-2-hydroxy-cyclohexyl)-amide,
6-(3,4-dichloro-phenyl)-5-phenoxy-3-trifluoromethyl-pyrazine-2-carboxylic acid (2-cyclopropyl-2-hydroxy-propyl)-amide,
6-(3,4-dichloro-phenyl)-5-(pyridin-2-ylmethoxy)-3-trifluoromethyl-pyrazine-2-carboxylic acid ((1R,2R)-2-hydroxy-cyclohexyl)-amide,
6-(3,4-dichloro-phenyl)-5-(pyridin-2-ylmethoxy)-3-trifluoromethyl-pyrazine-2-carboxylic acid (2-cyclopropyl-2-hydroxy-propyl)-amide,
6-(4-methanesulfonylamino-phenyl)-5-(2,2,2-trifluoro-ethoxy)-3-trifluoromethyl-pyrazine-2-carboxylic acid (1R,2R)-2-hydroxy-cyclohexyl)-amide,
6-(4-methanesulfonylamino-phenyl)-5-(2,2,2-trifluoro-ethoxy)-3-trifluoromethyl-pyrazine-2-carboxylic acid (2-cyclopropyl-2-hydroxy-propyl)-amide,
6-(4-methanesulfonylamino-phenyl)-5-cyclopropylmethoxy-3-trifluoromethyl-pyrazine-2-carboxylic acid (1R,2R)-2-hydroxy-cyclohexyl)-amide,
6-(4-methanesulfonylamino-phenyl)-5-cyclopropylmethoxy-3-trifluoromethyl-pyrazine-2-carboxylic acid (2-cyclopropyl-2-hydroxy-propyl)-amide,
6-(4-methanesulfonylamino-phenyl)-5-phenoxy-3-trifluoromethyl-pyrazine-2-carboxcylic acid ((1R,2R)-2-hydroxy-cyclohexyl)-amide,
6-(4-methanesulfonylamino-phenyl)-5-phenoxy-3-trifluoromethyl-pyrazine-2-carboxylic acid (2-cyclopropyl-2-hydroxy-propyl)-amide,
6-(4-methanesulfonylamino-phenyl)-5-(pyridin-2-ylmethoxy)-3-trifluoromethyl-pyrazine-2-carboxylic acid ((1R,2R)-2-hydroxy-cyclohexyl)-amide, and
6-(4-methanesulfonylamino-phenyl)-5-(pyridin-2-ylmethoxy)-3-trifluoromethyl-pyrazine-2-carboxylic acid (2-cyclopropyl-2-hydroxy-propyl)-amide.

Additional compounds for use in the present invention are disclosed in U.S. Pat. No. 7,572,823 including a class of compounds according to Formula 9:

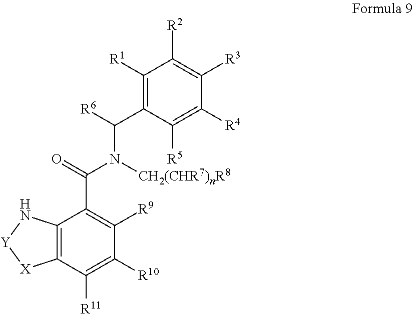

wherein —X—Y— is —CR^{a}═CR^{c}— or —CR^{a}═N— or —CR^{a}R^{b}—CR^{c}R^{d}—,
R^{a}, R^{b}, R^{c} and R^{d} are independently from each other selected from the group consisting of hydrogen and C₁-C₈ alkyl;
R¹, R², R⁴ and R⁵ are independently from each other selected from the group consisting of hydrogen, C₁-C₈ alkyl, C₁-C₈ alkoxy, halogen and halogen-C₁-C₈ alkyl;

### R³ is Si(CH₃)₃ or Si(CH₃)₂CH(CH₃)₂;

R⁶ is selected from the group consisting of hydrogen and C₁-C₈ alkyl;
R⁷ is selected from the group consisting of hydrogen, C₁-C₈ alkyl, hydroxy and halogen;
R⁸ is selected from the group consisting of C₁-C₈ alkyl, C₂-C₈ alkenyl, halogen-C₁-C₈ alkyl, heterocyclyl, heteroaryl which is unsubstituted or substituted by one or two groups independently selected from C₁-C₈ alkyl, C₁-C₈ alkoxy, halogen-C₁-C₈ alkyl, halogen-C₁-C₈ alkoxy and halogen, phenyl which is unsubstituted or substituted by one or two groups independently selected from C₁-C₈ alkyl, C₁-C₈ alkoxy, halogen-C₁-C₈ alkyl, halogen-C₁-C₅ alkoxy and halogen, —OR₁₂, wherein R₁₂ is C₁-C₈ alkyl or phenyl which is unsubstituted or substituted by one or two groups independently selected from C₁-C₈ alkyl, C₁-C₈ alkoxy, halogen-C₁-C₈ alkyl, halogen-C₁-C₈alkoxy and halogen, —NR¹³R¹⁴, wherein R¹³ and R¹⁴ independently from each other are selected from hydrogen, C₁-C₈ alkyl, and phenyl which is unsubstituted or substituted by one or two groups independently selected from C₁-C₈ alkyl, C₁-C₈ alkoxy, halogen-C₁-C₈ alkyl, halogen-C₁-C₈alkoxy and halogen, and —C(O)—OR¹⁵, wherein R¹⁵ is hydrogen or C₁-C₈ alkyl; R⁹, R¹⁰ and R¹¹ independently from each other are selected from the group consisting of hydrogen, C₁-C₈ alkyl, cycloalkyl, C₁-C₈ alkoxy, halogen-C₁-C₈ alkyl, and halogen; and n is 1, 2 or 3.

In particular, compounds of Formula 9 as described in paragraph [00167] above useful in the present invention are those wherein:

R⁸ is heterocyclyl or heteroaryl which is unsubstituted or substituted by one or two groups independently selected from C₁-C₈ alkyl, C₁-C₈ alkoxy, halogen-C₁-C₈ alkyl, halogen-C₁-C₈ alkoxy and halogen; or
R⁸ is —OR¹², and R¹² is C₁-C₈ alkyl or phenyl which is unsubstituted or substituted by one or two groups independently selected from C₁-C₈ alkyl, C₁-C₈ alkoxy, halogen-C₁-C₈ alkyl, halogen-C₁-C₈ alkoxy and halogen; or
R⁸ is —NR¹³R¹⁴, wherein R¹³ and R¹⁴ independently from each other are selected from hydrogen, C₁-C₈ alkyl, and phenyl which is unsubstituted or substituted by one or two groups independently selected from C₁-C₈ alkyl, C₁-C₈ alkoxy, halogen-C₁-C₈ alkyl, halogen-C₁-C₈ alkoxy and halogen; or
R⁸ is C(O)—OR¹⁵, wherein R¹⁵ is hydrogen or C₁-C₈ alkyl; or
R⁸ is phenyl which is unsubstituted or substituted by one or two groups independently selected from C₁-C₈ alkyl, C₁-C₈ alkoxy, halogen-C₁-C₈ alkyl, halogen-C₁-C₈ alkoxy and halogen.

### EXAMPLES

Studies of the effect of the infustion of 2 types of apolipoprotein A-I complexes (APLC-I and APLC-2) on left ventricular diastolic dysfunction were performed in an animal model.

### Experimental Approach

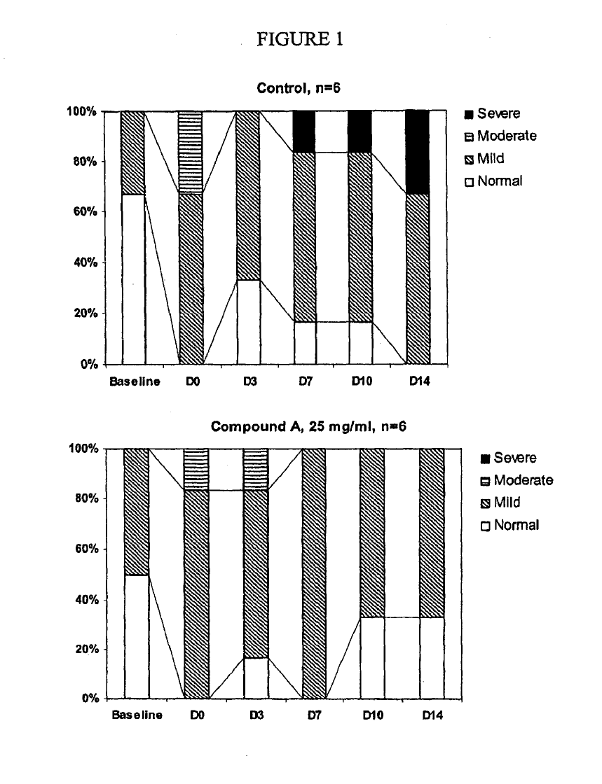

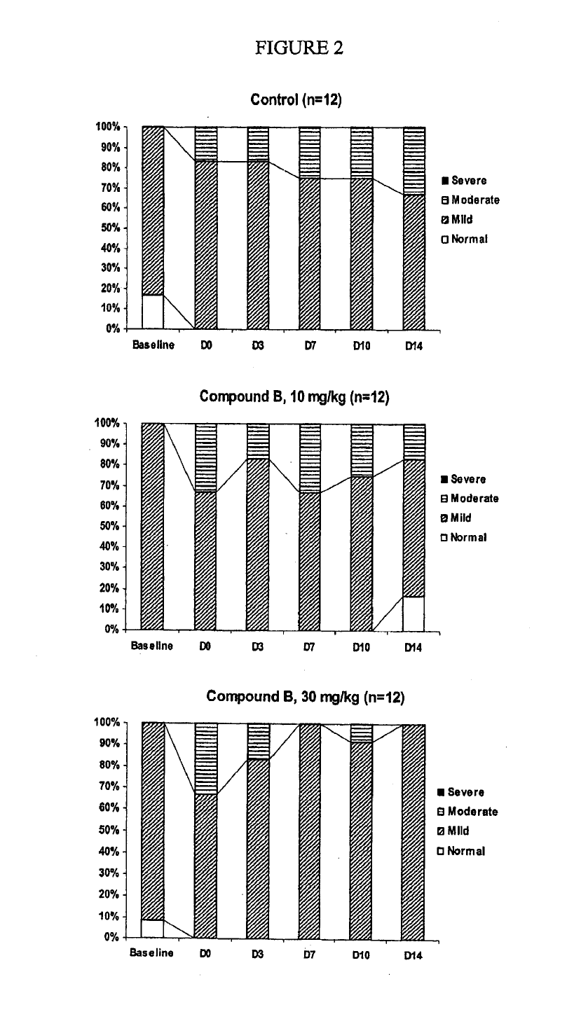

Forty-eight New-Zealand White male rabbits received a cholesterol-enriched diet and vitamin D₂ until significant decrease (>10%) in aortic valve area could be detected by echocardiography for each rabbit. At this point, rabbits showed mild to moderate diastolic dysfunction (See the time point DO in FIGS. 1 and 2). The enriched diet was then stopped to mimic cholesterol-lowering therapy.

Animals were randomized in a first experiment to receive: saline (control group, n=6) or APLC-1 at 25 mg/kg (treated group, n=6) whereas in a second experiment the control group received phosphate buffered saline (n=12) or APLC-2 at 10 or 30 mg/kg (treated groups, n=12 for each group). In both experiments, the treatment was administered 3 times per week for 2 weeks.

At day 3, 7, and 10 after initiation of the therapy and one day before sacrifice (D14), left ventricular diastolic dysfunction was studied using transthoracic echocardiography and classified either as normal, mild, moderate or severe dysfunction based on established criteria.

### Preparation of Apolipoprotein A-I Complexes

The protein fraction of APLC-I contained the Apo A-I analogue peptide: H-Pro-Val-Leu-Asp-Leu-Phe-Arg-Glu-Leu-Leu-Asn-Glu-Leu-Leu-Glu-Ala-Leu-Lys-Gln-Lys-Leu-Lys-OH (SEQ ID NO. 56). The peptide according to SEQ ID NO. 56 was obtained from Polypeptide Laboratories (Torrance, Calif., USA), and its purity assessed by high performance liquid chromatography (HPLC) and mass spectral analysis was greater than 98%. The APLC-I peptide/lipid complex was prepared by mixing the peptide with egg sphingomyelin (SPH) and 1,2-dipalmitoyl-sn-glycero-3-phosphocholine (DPPC) (Avanti Polar Lipids. Alabaster, Ala., USA) in a 1:1:1 weight ratio by mixing the components in saline and performing multiple heating and cooling cycles until the solution appeared perfectly clear. Fresh solution was prepared every week under sterile conditions and kept at 4° C.

The protein fraction of APLC-2 contained the Apo A-I analogue peptide: H-Lys-Leu-Lys-Gln-Lys₅-Leu-Ala-Glu-Leu-Leu10-Glu-Asn-Leu-Leu-Glu₁₅-Arg-Phe-Leu-Asp-Leu₂₀-Val-Inp₂₂-OH (SEQ ID NO. 116). This peptide is capped at the C-terminal end with isonipecotic acid, a proline analog. The peptide (SEQ ID NO. 116) was prepared by standard f-moc chemical synthesis and purified by reverse phase HPLC. APLC-2 was prepared by incorporating the peptide with phospholipids in a 1:2.5 (w/w) ratio using SPH, DPPC and 1,2-dipalmitoyl-sn-glycero-3-[phospho-rac-(1-glycerol)](DPPG). The lipid composition of the complexes is 48.5% SPH/48.5% DPPC/3% DPPG (w/w/w). The peptide/phospholipid complex was prepared using methods known in the art

### Results

### Example 1

For the first experiment, at the end of the treatment, left ventricular diastolic filling patterns were distributed differently among groups (P=0.018). Left ventricular diastolic dysfunction (LVDD) was attenuated by APLC-I infusions (33.3% of normal LVDD and 66.6% of mild DD vs. 66.6% of mild LVDD and 33.3% of severe LVDD for control rabbits). Infusions of APLC-I lead to reduction of left ventricular DD in a hypercholesterolemic rabbit model.

### Results

### Example 2

For the second experiment, at the end of the treatment period, left ventricular diastolic filling patterns were distributed differently among groups (P=0.048). Left ventricular DD was attenuated by APLC-2 infusions (100% of mild LVDD in the 30 mg/kg APLC-2 group vs. 66.6% of mild LVDD and 33.3% of moderate LVDD for control rabbits). Infusions of APLC-2 lead to reduction of left ventricular DD in a hypercholesterolemic rabbit model.

### Methods—Animals and Experiments

### Animals and Experiments

Animal care and procedures complied with the Canadian Council on Animal Care guidelines and were approved by the Montreal Heart Institute's ethics committee for animal research.

Male New-Zealand White rabbits (2.7-3.0 kg, aged 12-13 weeks) were fed with a 0.5% cholesterol-enriched diet (Harlan, Indianapolis, Ind., USA) plus vitamin D₂ (50000 IU per day; Sigma, Markham, Canada) in the drinking water until a >10% decrease of aortic valve area (AVA) could be detected by echocardiography (as described in Busseuil D, Shi Y, Mecteau M, Brand G, Kernaleguen A E, Thorin E, Latour J G, Rhéaume E, Tardif J C (2008). Regression of aortic valve stenosis by ApoA-I mimetic peptide infusions in rabbits. Brit J Pharm 154(4):765-73, the contents of which is hereby incorporated by reference in its entirety).

The animals then returned to a standard diet (without vitamin D₂) to mimic cholesterol-lowering therapy and were randomized in a first experiment to receive saline (control group, n=6) or APLC-I at 25 mg/kg (treated groups, n=6) and in a second experiment the control group received phosphate buffered saline (n=12) or APLC-2 at 10 or 30 mg/kg (treated groups, n=12 for each group). In both experiments treatment was administered 3 times per week for 2 weeks as injections through the marginal ear vein.

### Echocardiography

Transthoracic echocardiographic studies were performed at baseline, on a weekly basis starting at 8 weeks of hypercholesterolemic diet until significant AVA decreased more than 10% and then after 4, 7, 10 and 14 days of APLC or saline control treatments. Studies were carried out with a phased-array probe 10S (4.5˜11.5 Megahertz) and a Vivid 7 Dimension system (GE Healthcare Ultrasound, Horten, Norway). Intra-muscular injections of ketamine (22.5-45 mg/kg) and midazolam (0.5-0.75 mg/kg) were used for sedation.

Left ventricular (LV) M-mode spectrum was obtained in parasternal long-axis view to measure LV diameters at both end cardiac diastole (LVDd) and systole (LVDs). LV fractional shortening was calculated as (LVDd−LVDs)/LVDd×100%. Teicholz method was employed to calculate LV volumes and LV ejection fraction (EF). Pulsed wave Doppler was used to evaluate transmitral flow (TMF) and pulmonary venous flow (PVF) in apical 4-chamber view. TMF was used to measure the peak velocities during early filling (E) and atrial filling (A) and to calculate the E/A ratio. PVF was used to measure the systolic flow (S), diastolic flow (D) and reversed atrial flow (Ar). LV basal lateral peak systolic velocities (Sm) and mitral annulus velocities during early filling (Em) and atrial filling (Am) were derived by tissue Doppler imaging (TDI). The time intervals from the end of Am to the beginning of Em (b), and from the beginning to the end of Sm (a) were also measured on lateral wall TDI.

Left ventricular diastolic dysfunction (LVDD) was classified according to published criteria (Khouri et al., 2004). To further evaluate LVDD, left atrium (LA) M-mode spectrum was obtained in parasternal long-axis view at the aortic valve level and LA dimensions were measured in both end cardiac diastole and systole. LA fractional shortening was calculated as (systolic dimension−diastolic dimension)/systolic dimension×100%. The average of 3 consecutive cardiac cycles was used for each measurement.

All echocardiographic imaging and measurements were performed throughout the protocol by the same experienced investigator blinded to randomized treatment assignment.

### Statistical Analyses

Diastolic dysfunction classification was compared across groups using either chi-square or Fisher's exact test. All analyses were done with SAS version 9.1 (SAS Institute Inc., Cary, N.C., USA) and conducted at the 0.05 significance level.

### Results

With reference to FIG. 1 which illustrates the effect of treatment with APLC-I, the distribution of the pattern of LVDD classification evolved differently in the control and treated groups. Whereas severe LVDD appeared in some control animals after 7 days of treatment, no moderate or severe LVDD could be detected in treated animals. At the end of the treatment, LV diastolic filling patterns were distributed differently among groups (P=0.018). Left ventricular diastolic dysfunction (LVDD) was attenuated by APLC-I infusions (33.3% of normal LVDD and 66.6% of mild LVDD vs. 66.6% of mild LVDD and 33.3% of severe LVDD for control rabbits).

With reference to the FIG. 2 which illustrates the effect of treatment with APLC-2, the distribution of the pattern of LVDD classification evolved differently in the control and treated groups. Whereas moderate LVDD increased during treatment in the control group, moderate LVDD was stable or decreased in the 10 mg/kg APLC-2 group or decreased and then no longer detectable after 14 days in the 30 mg/kg APLC-2 group as it was replaced by the mild LVDD pattern. Thus, at the end of the 2-week treatment, LV diastolic filling patterns were distributed differently among groups (P=0.048). Left ventricular diastolic dysfunction (LVDD) was attenuated by APLC-2 infusions (100% of mild LVDD vs. 66.6% of mild LVDD and 33.3% of moderate LVDD for control rabbits).

1. A pharmaceutical composition for the treatment of left ventricular diastolic dysfunction (LVDD) comprising a CETP inhibitor selected from:
propanethioic acid, 2-methyl-, S-[2-[[[1-(2-ethylbutyl)cyclohexyl]carbonyl]amino]phenyl]ester (Dalcetrapib),
S-[2-(1-isopentylcyclohexanecarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxy-thioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)-phenyl]2,2-dimethylthiopropionate;
S[4,5-dichloro-2-(1-isopentylcyclopentanecarbonylamino)-phenyl]2,2-dimethylthiopropionate;
S-[2-(1-isopentylcyclohexanecarbonylamino)-4-trifluoromethylphenyl]2,2-dimethylthiopropionate;
O-methyl S-[2-(1-isopentylcyclohexanecarbonylaminophenyl monothiocarbonate;
S-[2-(1-methylcyclohexanecarbonylamino)phenyl]S-phenyldithiocarbonate;
S-[2-(1-isopentylcyclohexanecarbonylamino)phenyl]N-phenylthiocarbamate;
S-[2-(pivaloylamino)-4-trifluoromethylphenyl]2,2-dimethylthiopropionate;
S-[4,5-dichloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate;
S-[4,5-dichloro-2-(1-pentylcyclohexanecarbonylamino)-phenyl]2,2-dimethylthiopropionate;
S-[4,5-dichloro-2-(1-cyclopropylmethylcyclohexanecarbonylamino)phenyl]2,2-dimethylthiopropionate;
S-[4,5-dichloro-2-(1-cyclohexylmethylcyclohexanecarbonylamino)phenyl]2,2-dimethylthiopropionate;
S-[4,5-dichloro-2-(1-isopropylcyclohexanecarbonylamino)-phenyl]2,2-dimethylthiopropionate;
S-[4,5-dichloro-2-(1-isopentylcycloheptanecarbonylamino)-phenyl]2,2-dimethylthiopropionate;
S-[4,5-dichloro-2-(1-isopentylcyclobutanecarbonylamino)-phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethylthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)-phenyl]2,2-dimethylthiopropionate;
S-[5-fluoro-2-(1-isopentylcyclohexanecarbonylamino)phenyl]2,2-dimethylthiopropionate;
bis-[4,5-dichloro-2-(1-isopentylcyclohexanecarbonylamino)-phenyl]disulfide;
2-tetrahydrofurylmethyl-2-(1-isopentylcyclohexanecarbonylamino) phenyl disulfide;
N-(2-mercaptophenyl)-1-ethylcyclohexanecarboxamide;
N-(2-mercaptophenyl)-1-propylcyclohexanecarboxamide;
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]thioacetate;
bis-[2-[1-(2-ethylbutyl)cyclohexanecarbonylamino]phenyl]disulfide;
N-(2-mercaptophenyl)-1-(2-ethylbutyl)cyclohexanecarboxamide;
S[2-[1-(2-ethylbutyl)cyclohexanecarbonylamino]phenyl]2-methylthiopropionate;
S-[2-(1-isobutylcyclohexanecarbonylamino)phenyl]2-methylthiopropionate;
S-[2-[1-(2-ethylbutyl)cyclohexanecarbonylamino]phenyl]-acetylpiperidine-4-thiocarboxylate;
S-[2-[1-(2-ethylbutyl)cyclohexanecarbonylamino]phenyl]thioacetate;
S-[2-[1 (2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-[1-(2-ethylbutyl)cyclohexanecarbonylamino]phenyl]2-hydroxy-2-methylthiopropionate;
S-[2-[1-(2-ethylbutyl)cyclohexanecarbonylamino]phenyl]4-chlorophenoxythioacetate;
S-[2-(1-isobutylcyclohexanecarbonylamino)phenyl]4-chlorophenoxythioacetate; and
S-[2-(1-isobutylcyclohexanecarbonylamino)phenyl]-1-acetyl-piperidine-4-thiocarboxylate.

2.-37. (canceled)

38. The composition of claim 1 comprising a pro-drug compound that delivers a therapeutic amount of a CETP inhibitor in vivo.

39. The composition of claim 1 comprising a pharmaceutically acceptable salt of a CETP inhibitor.

40. The composition of claim 1 comprising a pharmaceutically acceptable hydrate of CETP inhibitor.

41. The composition of claim 1 comprising a pharmaceutically acceptable solvate of CETP inhibitor.

42. The composition of claim 1 formulated for oral administration at a dose for an adult between 1 and 100 mg per day.

43. The composition of claim 1 formulated for oral administration at a dose for an adult between 50 and 800 mg per day.


SEQUENCE LISTING (174 sequences)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 4 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 5 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 6 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 7 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 8 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPRGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 9 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPRGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 10 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 11 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNT

SEQ ID NO: 12 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPRGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 13 - protein, 100 aa, Homo sapiens
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 14 - protein, 396 aa, Homo sapiens [sequence omitted]

SEQ ID NO: 15 - protein, 366 aa, Homo sapiens
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 16 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 17 - protein, 83 aa, Homo sapiens
MRLFLSLPVLVVVLSIVLEGPAPAQGTPDVSSALDKLKEFGNTLEDKARELISRIKQSELSAKMREWFSETFQKVKEKLKIDS

SEQ ID NO: 18 - protein, 101 aa, Homo sapiens
MGTRLLPALFLVLLVLGFEVQGTQQPQQDEMPSPTFLTQVKESLSSYWESAKTAAQNLYEKTYLPAVDEKLRDLYSKSTAAMSTYTGIFTDQVLSVLKGEE

SEQ ID NO: 19 - protein, 99 aa, Homo sapiens
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 20 - protein, 189 aa, Homo sapiens
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 21 - protein, 317 aa, Homo sapiens [sequence omitted]

SEQ ID NO: 22 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 23 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 24 - protein, 440 aa, Homo sapiens
MGPPGSPWQWVTLLLGLLLPPAAPFWLLNVLFPPHTTPKAELSNHTRPVILVPGCLGNQLEAKLDKPDVVNWMCYRKTEDFFTIWLDLNMFLPLGVDCWIDNTRVVYNRSSGLVSNAPGVQIRVPGFGKTYSVEYLDSSKLAGYLHTLVQNLVNNGYVRDETVRAAPYDWRLEPGQQEEYYRKLAGLVEEMHAAYGKPVFLIGHSLGCLHLLYFLLRQPQAWKDRFIDGFISLGAPWGGSIKPMLVLASGDNQGIPIMSSIKLKEEQRITTTSPWMFPSRMAWPEDHVFISTPSFNYTGRDFQRFFADLHFEEGWYMWLQSRDLLAGLPAPGVEVYCLYGVGLPTPRTYIYDHGFPYTDPVGVLYEDGDDTVATRSTELCGLWQGRQPQPVHLLPLHGIQHLNMVFSNLTLEHINAILLGAYRQGPPASPTASPEPPPPE

SEQ ID NO: 25 - protein, 493 aa, Homo sapiens [sequence omitted]

SEQ ID NO: 26 - protein, 493 aa, Homo sapiens
MALFGALFLALLAGAHAEFPGCKIRVTSKALELVKQEGLRFLEQELETITIPDLRGKEGHFYYNISEVKVTELQLTSSELDFQPQQELMLQITNASLGLRFRRQLLYWFFYDGGYINASAEGVSIRTGLELSRDPAGRMKVSNVSCQASVSRMHAAFGGTFKKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 27 - protein, 355 aa, Homo sapiens [sequence omitted]

SEQ ID NO: 28 - protein, 242 aa, Homo sapiens
DEHPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 29 - protein, 242 aa, Homo sapiens
DERPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 30 - protein, 242 aa, Homo sapiens
DEPRQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 31 - protein, 242 aa, Homo sapiens
DEPPQSPWDLVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 32 - protein, 242 aa, Homo sapiens
DEPPQSPWDRVKDLATVYVDVLKDSRRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 33 - protein, 242 aa, Homo sapiens
DEPPQSPWDRVKDLATVYVDVLKDSGRDYVSQFEGSTLGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 34 - protein, 242 aa, Homo sapiens
DEPPQSPWDRVKDLATVYVDVLKDSGRDYVSQFEGSALGKQLNLKLLDNWDSVTSTFSKRREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 35 - protein, 242 aa, Homo sapiens
DEPPQSPWDRVKDLATVYVDVLKDSGRDYVSQFEGSALGKQLNLKLLDNWDSVTSTFSKLREQLGPVIQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 36 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKELEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 37 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKDKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 38 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLHDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 39 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDNFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 40 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKMWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 41 - protein, 241 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 42 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKRQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 43 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQKEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 44 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHKLQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 45 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQGKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 46 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSRLGEEMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 47 - skipped

SEQ ID NO: 48 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEVMRDRARAHVDALRTHLAPYSDELRQRLAARLEALKENGGARLAEYHAKATEHLSTLSEKAKPALEDLRQGLLPVLESFKVSFLSALEEYTKKLNT

SEQ ID NO: 49 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHEDALRTHLAPYSDELRQRLAARLEALKENGGARLAEYHAKATEHLSTLSEKAKPALEDLRQGLLPVLESFKVSFLSALEEYTKKLNT

SEQ ID NO: 50 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDAPRTHLAPYSDELRQRLAARLEALKENGGARLAEYHAKATEHLSTLSEKAKPALEDLRQGLLPVLESFKVSFLSALEEYTKKLNT

SEQ ID NO: 51 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALPTHLAPYSDELRQRLAARLEALKENGGARLAEYHAKATEHLSTLSEKAKPALEDLRQGLLPVLESFKVSFLSALEEYTKKLNT

SEQ ID NO: 52 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLARYSDELRQRLAARLEALKENGGARLAEYHAKATEHLSTLSEKAKPALEDLRQGLLPVLESFKVSFLSALEEYTKKLNT

SEQ ID NO: 53 - protein, 242 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATKHLSTLSEKAKPALEDLRQGLLPVLESFKVSFLSALEEYTKKLNT

SEQ ID NO: 54 - protein, 23 aa, Homo sapiens
GVLDLFRELLNELLEALKQKLKK

SEQ ID NO: 55 - protein, 22 aa, Homo sapiens
PVLDLFRELLNELLEWLKQKLK

SEQ ID NO: 56 - protein, 22 aa, Homo sapiens
PVLDLFRELLNELLEALKQKLK

SEQ ID NO: 57 - protein, 23 aa, Homo sapiens
PVLDLFRELLNELLEALKQKLKK

SEQ ID NO: 58 - protein, 22 aa, Homo sapiens
  misc_feature (13)..(13): Xaa can be any naturally occurring amino acid
PVLDLFRELLNEXLEALKQKLK

SEQ ID NO: 59 - protein, 22 aa, Homo sapiens
PVLDLFKELLNELLEALKQKLK

SEQ ID NO: 60 - protein, 22 aa, Homo sapiens
PVLDLFRELLNEGLEALKQKLK

SEQ ID NO: 61 - protein, 22 aa, Homo sapiens
PVLDLFRELGNELLEALKQKLK

SEQ ID NO: 62 - protein, 22 aa, Homo sapiens
PVLDLFRELLNELLEAZKQKLK

SEQ ID NO: 63 - protein, 22 aa, Homo sapiens
PVLDLFKELLQELLEALKQKLK

SEQ ID NO: 64 - protein, 22 aa, Homo sapiens
PVLDLFRELLNELLEAGKQKLK

SEQ ID NO: 65 - protein, 22 aa, Artificial Sequence
  Synthetic Construct
GVLDLFRELLNEGLEALKQKLK

SEQ ID NO: 66 - protein, 22 aa, Homo sapiens
  MISC_FEATURE (18)..(18): Xaa is Ornithine (Orn)
  MISC_FEATURE (20)..(20): Xaa is Ornithine (Orn)
  MISC_FEATURE (22)..(22): Xaa is Ornithine (Orn)
PVLDLFRELLNELLEALXQXLX

SEQ ID NO: 67 - protein, 22 aa, Homo sapiens
PVLDLFRELWNELLEALKQKLK

SEQ ID NO: 68 - protein, 22 aa, Homo sapiens
PVLDLLRELLNELLEALKQKLK

SEQ ID NO: 69 - protein, 22 aa, Homo sapiens
PVLELFKELLQELLEALKQKLK

SEQ ID NO: 70 - protein, 22 aa, Homo sapiens
GVLDLFRELLNELLEALKQKLK

SEQ ID NO: 71 - protein, 22 aa, Homo sapiens
PVLDLFRELLNEGLEALKQKLK

SEQ ID NO: 72 - protein, 22 aa, Homo sapiens
PVLDLFREGLNELLEALKQKLK

SEQ ID NO: 73 - protein, 22 aa, Homo sapiens
PVLDLFRELLNELLEALKQKLK

SEQ ID NO: 74 - protein, 22 aa, Homo sapiens
PVLDLFRELLNELLEGLKQKLK

SEQ ID NO: 75 - protein, 22 aa, Homo sapiens
PLLELFKELLQELLEALKQKLK

SEQ ID NO: 76 - protein, 22 aa, Homo sapiens
PVLDLFRELLNELLEALQKKLK

SEQ ID NO: 77 - protein, 22 aa, Homo sapiens
  misc_feature (13)..(13): Xaa can be any naturally occurring amino acid
PVLDFFRELLNEXLEALKQKLK

SEQ ID NO: 78 - protein, 22 aa, Homo sapiens
PVLDLFRELLNELLELLKQKLK

SEQ ID NO: 79 - protein, 22 aa, Homo sapiens
PVLDLFRELLNELZEALKQKLK

SEQ ID NO: 80 - protein, 22 aa, Homo sapiens
PVLDLFRELLNELWEALKQKLK

SEQ ID NO: 81 - protein, 22 aa, Homo sapiens
AVLDLFRELLNELLEALKQKLK

SEQ ID NO: 82 - protein, 22 aa, Homo sapiens
QVLDLFRELLNELLEALKQKLK

SEQ ID NO: 83 - protein, 22 aa, Homo sapiens
  MISC_FEATURE (7)..(7): Xaa is ornithine (Orn)
  MISC_FEATURE (18)..(18): Xaa is ornithine (Orn)
  MISC_FEATURE (20)..(20): Xaa is ornithine (Orn)
  MISC_FEATURE (22)..(22): Xaa is ornithine (Orn)
PVLDLFXELLNELLEALXQXLX

SEQ ID NO: 84 - protein, 22 aa, Homo sapiens
NVLDLFRELLNELLEALKQKLK

SEQ ID NO: 85 - protein, 22 aa, Homo sapiens
PVLDLFRELLNELGEALKQKLK

SEQ ID NO: 86 - protein, 22 aa, Homo sapiens
PVLDLFRELLNELLELLKQKLK

SEQ ID NO: 87 - protein, 22 aa, Homo sapiens
PVLDLFRELLNELLEFLKQKLK

SEQ ID NO: 88 - protein, 22 aa, Homo sapiens
PVLELFNDLLRELLEALQKKLK

SEQ ID NO: 89 - protein, 22 aa, Homo sapiens
PVLELFNDLLRELLEALKQKLK

SEQ ID NO: 90 - protein, 22 aa, Homo sapiens
PVLELFKELLNELLDALRQKLK

SEQ ID NO: 91 - protein, 22 aa, Homo sapiens
PVLDLFRELLENLLEALQKKLK

SEQ ID NO: 92 - protein, 22 aa, Homo sapiens
PVLELFERLLEDLLQALNKKLK

SEQ ID NO: 93 - protein, 22 aa, Homo sapiens
PVLELFERLLEDLLKALNQKLK

SEQ ID NO: 94 - protein, 22 aa, Homo sapiens
DVLDLFRELLNELLEALKQKLK

SEQ ID NO: 95 - protein, 22 aa, Homo sapiens
PALELFKDLLQELLEALKQKLK

SEQ ID NO: 96 - protein, 22 aa, Homo sapiens
PVLDLFRELLNEGLEAZKQKLK

SEQ ID NO: 97 - protein, 22 aa, Homo sapiens
PVLDLFRELLNEGLEWLKQKLK

SEQ ID NO: 98 - protein, 22 aa, Homo sapiens
PVLDLFRELWNEGLEALKQKLK

SEQ ID NO: 99 - protein, 22 aa, Homo sapiens
  MISC_FEATURE (18)..(18): Xaa is ornithine (Orn)
  MISC_FEATURE (20)..(20): Xaa is ornithine (Orn)
  MISC_FEATURE (22)..(22): Xaa is ornithine (Orn)
PVLDLFRELLNEGLEALXQXLX

SEQ ID NO: 100 - protein, 22 aa, Homo sapiens
PVLDFFRELLNEGLEALQKKLK

SEQ ID NO: 101 - protein, 22 aa, Homo sapiens
PVLELFRELLNEGLEALKQKLK

SEQ ID NO: 102 - protein, 22 aa, Artificial Sequence
  Synthetic Construct
  MISC_FEATURE (22)..(22): Xaa is non-natural amino acid ′′isonipecotic acid′′ (Inp)
KLKQKLWELLENLLERFLDLVX

SEQ ID NO: 103 - protein, 22 aa, Artificial Sequence
  Synthetic Construct
  MISC_FEATURE (22)..(22): Xaa is the non-natural amino acid isonipecotic acid (Inp)
KLKQKLAELGENLLERFLDLVX

SEQ ID NO: 104 - protein, 22 aa, Artificial Sequence
  Synthetic Construct
  MISC_FEATURE (22)..(22): Xaa is the non-natural amino acid isonipecotic acid (Inp)
KLKQKLAELGENLLERFLDLVX

SEQ ID NO: 105 - protein, 22 aa, Artificial Sequence
  Synthetic Construct
  MISC_FEATURE (22)..(22): Xaa is the non-natural amino acid isonipecotic acid (Inp)
KLKQKLWELGENLLERFLDLVX

SEQ ID NO: 106 - protein, 22 aa, Artificial Sequence
  Synthetic Construct
  MISC_FEATURE (22)..(22): Xaa is the non-natural amino acid isonipecotic acid (Inp)
KLKQKLAELGENWLERFLDLVX

SEQ ID NO: 107 - protein, 22 aa, Artificial Sequence
  Synthetic Construct
  MISC_FEATURE (1)..(1): Xaa is Orn
  MISC_FEATURE (3)..(3): Xaa is Orn
  MISC_FEATURE (5)..(5): Xaa is Orn
  MISC_FEATURE (22)..(22): Xaa is the non-natural amino acid isonipecotic acid (Inp)
XLXQXLAELGENLLERFLDLVX

SEQ ID NO: 108 - protein, 22 aa, Artificial Sequence
  Synthetic Construct
  MISC_FEATURE (22)..(22): Xaa is the non-natural amino acid isonipecotic acid (Inp)
KLKQKLAELGENLLERFFDLVX

SEQ ID NO: 109 - protein, 22 aa, Artificial Sequence
  Synthetic Construct
  MISC_FEATURE (22)..(22): Xaa is the non-natural amino acid isonipecotic acid (Inp)
KLKQKLAELGENLLERFLELVX

SEQ ID NO: 110 - protein, 22 aa, Artificial Sequence
  Synthetic Construct
  MISC_FEATURE (22)..(22): Xaa is the non-natural amino acid isonipecotic acid (Inp)
KLKQKLAELLENLGERFLDLVX

SEQ ID NO: 111 - protein, 22 aa, Artificial Sequence
  Synthetic Construct
  MISC_FEATURE (22)..(22): Xaa is the non-natural amino acid isonipecotic acid (Inp)
KLKQKLAELLENGLERFLDLVX

SEQ ID NO: 112 - protein, 22 aa, Artificial Sequence
  Synthetic Construct
  MISC_FEATURE (22)..(22): Xaa is the non-natural amino acid isonipecotic acid (Inp)
KLKQKLAEGLENLLERFLDLVX

SEQ ID NO: 113 - protein, 22 aa, Artificial Sequence
  Synthetic Construct
  MISC_FEATURE (22)..(22): Xaa is the non-natural amino acid isonipecotic acid (Inp)
KLKQKLGELLENLLERFLDLVX

SEQ ID NO: 114 - protein, 22 aa, Artificial Sequence
  Synthetic Construct
  MISC_FEATURE (22)..(22): Xaa is the non-natural amino acid isonipecotic acid (Inp)
KLKQKGAELLENLLERFLDLVX

SEQ ID NO: 115 - protein, 22 aa, Artificial Sequence
  Synthetic Construct
  MISC_FEATURE (7)..(7): Xaa is the non-natural amino acid naphthylalanine (Nal)
  MISC_FEATURE (22)..(22): Xaa is the non-natural amino acid isonipecotic acid (Inp)
KLKQKLXELLENLLERFLDLVX

SEQ ID NO: 116 - protein, 22 aa, Artificial Sequence
  Synthetic Construct
  MISC_FEATURE (22)..(22): Xaa is the non-natural amino acid isonipecotic acid (Inp)
KLKQKLAELLENLLERFLDLVX

SEQ ID NO: 117 - protein, 22 aa, Artificial Sequence
  Synthetic Construct
  MISC_FEATURE (10)..(10): Xaa is the non-natural amino acid alpha-aminoisobutyric acid (Aib)
  MISC_FEATURE (22)..(22): Xaa is the non-natural amino acid isonipecotic acid (Inp)
KLKQKLAELXENLLERFLDLVX

SEQ ID NO: 118 - protein, 22 aa, Artificial Sequence
  Synthetic Construct
  MISC_FEATURE (22)..(22): Xaa is the non-natural amino acid isonipecotic acid (Inp)
KLKQKLAELLENLLEKFLDLVX

SEQ ID NO: 119 - protein, 22 aa, Artificial Sequence
  Synthetic Construct
  MISC_FEATURE (6)..(6): Xaa is the non-natural amino acid naphthylalanine (Nal)
  MISC_FEATURE (22)..(22): Xaa is the non-natural amino acid isonipecotic acid (Inp)
KLKQKXAELLENLLERFLDLVX

SEQ ID NO: 120 - protein, 22 aa, Artificial Sequence
  Synthetic Construct
  MISC_FEATURE (22)..(22): Xaa is the non-natural amino acid isonipecotic acid (Inp)
KLKQKLAELLEQLLEKFLDLVX

SEQ ID NO: 121 - protein, 22 aa, Artificial Sequence
  Synthetic Construct
  MISC_FEATURE (1)..(1): Xaa is the non-natural amino acid ornithine (orn)
  MISC_FEATURE (3)..(3): Xaa is the non-natural amino acid ornithine (orn)
  MISC_FEATURE (5)..(5): Xaa is the non-natural amino acid ornithine (orn)
  MISC_FEATURE (22)..(22): Xaa is the non-natural amino acid isonipecotic acid (Inp)
XLXQXLAELLENLLERFLDLVX

SEQ ID NO: 122 - protein, 22 aa, Artificial Sequence
  Synthetic Construct
  MISC_FEATURE (22)..(22): Xaa is the non-natural amino acid isonipecotic acid (Inp)
KLKQKLAELLENWLERFLDLVX

SEQ ID NO: 123 - protein, 22 aa, Artificial Sequence
  Synthetic Construct
  MISC_FEATURE (22)..(22): Xaa is the non-natural amino acid isonipecotic acid (Inp)
KLKQKLAELLENLLERLLDLVX

SEQ ID NO: 124 - protein, 22 aa, Artificial Sequence
  Synthetic Construct
  MISC_FEATURE (22)..(22): Xaa is the non-natural amino acid isonipecotic acid (Inp)
KLKQKLAELLEQLLEKFLELVX

SEQ ID NO: 125 - protein, 22 aa, Artificial Sequence
  Synthetic Construct
  MISC_FEATURE (22)..(22): Xaa is the non-natural amino acid isonipecotic acid (Inp)
KLKQKLAELLEQLLEKFLELLX

SEQ ID NO: 126 - protein, 22 aa, Artificial Sequence
  Synthetic Construct
  MISC_FEATURE (10)..(10): Xaa is the non-natural amino acid alpha-aminoisobutyric acid (Aib)
  MISC_FEATURE (22)..(22): Xaa is the non-natural amino acid isonipecotic acid (Inp)
KLKQKLAELXENLLERFFDLVX

SEQ ID NO: 127 - protein, 22 aa, Artificial Sequence
  Synthetic Construct
  MISC_FEATURE (22)..(22): Xaa is the non-natural amino acid isonipecotic acid (Inp)
KLKQKLLELLENLLERFLDLVX

SEQ ID NO: 128 - protein, 22 aa, Artificial Sequence
  Synthetic Construct
  MISC_FEATURE (9)..(9): Xaa is the non-natural amino acid naphthylalanine (Nal)
  MISC_FEATURE (22)..(22): Xaa is the non-natural amino acid isonipecotic acid (Inp)
KLKQKLAEXLENLLERFLDLVX

SEQ ID NO: 129 - protein, 22 aa, Artificial Sequence
  Synthetic Construct
  MISC_FEATURE (22)..(22): Xaa is the non-natural amino acid isonipecotic acid (Inp)
KLKQKLAEWLENLLERFLDLVX

SEQ ID NO: 130 - protein, 22 aa, Artificial Sequence
  Synthetic Construct
  MISC_FEATURE (1)..(1): Xaa is the non-natural amino acid ornithine (orn)
  MISC_FEATURE (3)..(3): Xaa is the non-natural amino acid ornithine (orn)
  MISC_FEATURE (5)..(5): Xaa is the non-natural amino acid ornithine (orn)
  MISC_FEATURE (16)..(16): Xaa is the non-natural amino acid ornithine (orn)
  MISC_FEATURE (22)..(22): Xaa is the non-natural amino acid isonipecotic acid (Inp)
XLXQXLAELLENLLEXFLDLVX

SEQ ID NO: 131 - protein, 22 aa, Artificial Sequence
  Synthetic Construct
  MISC_FEATURE (22)..(22): Xaa is the non-natural amino acid isonipecotic acid (Inp)
KLKQKLFELLENLLERFLDLVX

SEQ ID NO: 132 - protein, 22 aa, Artificial Sequence
  Synthetic Construct
  MISC_FEATURE (22)..(22): Xaa is the non-natural amino acid isonipecotic acid (Inp)
KLKQRLADLLENLLEKFLELVX

SEQ ID NO: 133 - protein, 22 aa, Artificial Sequence
  Synthetic Construct
  MISC_FEATURE (22)..(22): Xaa is the non-natural amino acid isonipecotic acid (Inp)
KLKQKLAELLEQLLDKFLELAX

SEQ ID NO: 134 - protein, 22 aa, Artificial Sequence
  Synthetic Construct
  MISC_FEATURE (22)..(22): Xaa is the non-natural amino acid nipecotic acid (Nip)
KLKQKLWELLENLLERFLDLVX

SEQ ID NO: 135 - protein, 22 aa, Artificial Sequence
  Synthetic Construct
  MISC_FEATURE (22)..(22): Xaa is the non-natural amino acid nipecotic acid (Nip)
KLKQKLAELGENLLERFLDLVX

SEQ ID NO: 136 - protein, 22 aa, Artificial Sequence
  Synthetic Construct
  MISC_FEATURE (6)..(6): Xaa  is the non-natural amino acid naphthylalanine (Nal)
  MISC_FEATURE (22)..(22): Xaa is the non-natural amino acid nipecotic acid (Nip)
KLKQKXAELGENLLERFLDLVX

SEQ ID NO: 137 - protein, 22 aa, Artificial Sequence
  Synthetic Construct
  MISC_FEATURE (22)..(22): Xaa is the non-natural amino acid nipecotic acid (Nip)
KLKQKLWELGENLLERFLDLVX

SEQ ID NO: 138 - protein, 22 aa, Artificial Sequence
  Synthetic Construct
  MISC_FEATURE (22)..(22): Xaa is the non-natural amino acid nipecotic acid (Nip)
KLKQKLAELGENWLERFLDLVX

SEQ ID NO: 139 - protein, 22 aa, Artificial Sequence
  Synthetic Construct
  MISC_FEATURE (1)..(1): Xaa is the non-natural amino acid ornithine (orn)
  MISC_FEATURE (3)..(3): Xaa is the non-natural amino acid ornithine (orn)
  MISC_FEATURE (5)..(5): Xaa is the non-natural amino acid ornithine (orn)
  MISC_FEATURE (22)..(22): Xaa is the non-natural amino acid nipecotic acid (Nip)
XLXQXLAELGENLLERFLDLVX

SEQ ID NO: 140 - protein, 22 aa, Artificial Sequence
  Synthetic Construct
  MISC_FEATURE (22)..(22): Xaa is the non-natural amino acid nipecotic acid (Nip)
KLKQKLAELGENLLERFFDLVX

SEQ ID NO: 141 - protein, 22 aa, Artificial Sequence
  Synthetic Construct
  MISC_FEATURE (22)..(22): Xaa is the non-natural amino acid nipecotic acid (Nip)
KLKQKLAELGENLLERFLELVX

SEQ ID NO: 142 - protein, 22 aa, Artificial Sequence
  Synthetic Construct
  MISC_FEATURE (22)..(22): Xaa is the non-natural amino acid nipecotic acid (Nip)
KLKQKLAELLENLGERFLDLVX

SEQ ID NO: 143 - protein, 22 aa, Artificial Sequence
  Synthetic Construct
  MISC_FEATURE (22)..(22): Xaa is the non-natural amino acid nipecotic acid (Nip)
KLKQKLAELLENGLERFLDLVX

SEQ ID NO: 144 - protein, 22 aa, Artificial Sequence
  Synthetic Construct
  MISC_FEATURE (22)..(22): Xaa is the non-natural amino acid nipecotic acid (Nip)
KLKQKLAEGLENLLERFLDLVX

SEQ ID NO: 145 - protein, 22 aa, Artificial Sequence
  Synthetic Construct
  MISC_FEATURE (22)..(22): Xaa is the non-natural amino acid nipecotic acid (Nip)
KLKQKLGELLENLLERFLDLVX

SEQ ID NO: 146 - protein, 22 aa, Artificial Sequence
  Synthetic Construct
  MISC_FEATURE (22)..(22): Xaa is the non-natural amino acid nipecotic acid (Nip)
KLKQKGAELLENLLERFLDLVX

SEQ ID NO: 147 - protein, 22 aa, Artificial Sequence
  Synthetic Construct
  MISC_FEATURE (7)..(7): Xaa is the non-natural amino acid naphthylalanine (Nal)
  MISC_FEATURE (22)..(22): Xaa is the non-natural amino acid nipecotic acid (Nip)
KLKQKLXELLENLLERFLDLVX

SEQ ID NO: 148 - protein, 22 aa, Artificial Sequence
  Synthetic Construct
  MISC_FEATURE (22)..(22): Xaa is the non-natural amino acid nipecotic acid (Nip)
KLKQKLAELLENLLERFLDLVX

SEQ ID NO: 149 - protein, 22 aa, Artificial Sequence
  Synthetic Construct
  MISC_FEATURE (10)..(10): Xaa is the non-natural amino acid alpha-aminoisobutyric acid (Aib)
  MISC_FEATURE (22)..(22): Xaa is the non-natural amino acid nipecotic acid (Nip)
KLKQKLAELXENLLERFLDLVX

SEQ ID NO: 150 - protein, 22 aa, Artificial Sequence
  Synthetic Construct
  MISC_FEATURE (22)..(22): Xaa is the non-natural amino acid nipecotic acid (Nip)
KLKQKLAELLENLLEKFLDLVX

SEQ ID NO: 151 - protein, 22 aa, Artificial Sequence
  Synthetic Construct
  MISC_FEATURE (6)..(6): Xaa is the non-natural amino acid naphthylalanine (Nal)
  MISC_FEATURE (22)..(22): Xaa is the non-natural amino acid nipecotic acid (Nip)
KLKQKXAELLENLLERFLDLVX

SEQ ID NO: 152 - protein, 22 aa, Artificial Sequence
  Synthetic Construct
  MISC_FEATURE (22)..(22): Xaa is the non-natural amino acid nipecotic acid (Nip)
KLKQKLAELLEQLLEKFLDLVX

SEQ ID NO: 153 - protein, 22 aa, Artificial Sequence
  Synthetic Construct
  MISC_FEATURE (1)..(1): Xaa is the non-natural amino acid ornithine (orn)
  MISC_FEATURE (3)..(3): Xaa is the non-natural amino acid ornithine (orn)
  MISC_FEATURE (5)..(5): Xaa is the non-natural amino acid ornithine (orn)
  MISC_FEATURE (22)..(22): Xaa is the non-natural amino acid nipecotic acid (Nip)
XLXQXLAELLENLLERFLDLVX

SEQ ID NO: 154 - protein, 22 aa, Artificial Sequence
  Synthetic Construct
  MISC_FEATURE (22)..(22): Xaa is the non-natural amino acid nipecotic acid (Nip)
KLKQKLAELLENWLERFLDLVX

SEQ ID NO: 155 - protein, 22 aa, Artificial Sequence
  Synthetic Construct
  MISC_FEATURE (22)..(22): Xaa is the non-natural amino acid nipecotic acid (Nip)
KLKQKLAELLENLLERLLDLVX

SEQ ID NO: 156 - protein, 22 aa, Artificial Sequence
  Synthetic Construct
  MISC_FEATURE (22)..(22): Xaa is the non-natural amino acid nipecotic acid (Nip)
KLKQKLAELLEQLLEKFLELVX

SEQ ID NO: 157 - protein, 22 aa, Artificial Sequence
  Synthetic Construct
  MISC_FEATURE (22)..(22): Xaa is the non-natural amino acid nipecotic acid (Nip)
KLKQKLAELLEQLLEKFLELLX

SEQ ID NO: 158 - protein, 22 aa, Artificial Sequence
  Synthetic Construct
  MISC_FEATURE (10)..(10): Xaa is the non-natural amino acid alpha-aminoisobutyric acid (Aib)
  MISC_FEATURE (22)..(22): Xaa is the non-natural amino acid nipecotic acid (Nip)
KLKQKLAELXENLLERFFDLVX

SEQ ID NO: 159 - protein, 22 aa, Artificial Sequence
  Synthetic Construct
  MISC_FEATURE (22)..(22): Xaa is the non-natural amino acid nipecotic acid (Nip)
KLKQKLLELLENLLERFLDLVX

SEQ ID NO: 160 - protein, 22 aa, Artificial Sequence
  Synthetic Construct
  MISC_FEATURE (9)..(9): Xaa is the non-natural amino acid naphthylalanine (Nal)
  MISC_FEATURE (22)..(22): Xaa is the non-natural amino acid nipecotic acid (Nip)
KLKQKLAEXLENLLERFLDLVX

SEQ ID NO: 161 - protein, 22 aa, Artificial Sequence
  Synthetic Construct
  MISC_FEATURE (22)..(22): Xaa is the non-natural amino acid nipecotic acid (Nip)
KLKQKLAEWLENLLERFLDLVX

SEQ ID NO: 162 - protein, 22 aa, Artificial Sequence
  Synthetic Construct
  MISC_FEATURE (1)..(1): Xaa is the non-natural amino acid ornithine (orn)
  MISC_FEATURE (3)..(3): Xaa is the non-natural amino acid ornithine (orn)
  MISC_FEATURE (5)..(5): Xaa is the non-natural amino acid ornithine (orn)
  MISC_FEATURE (16)..(16): Xaa is the non-natural amino acid ornithine (orn)
  MISC_FEATURE (22)..(22): Xaa is the non-natural amino acid nipecotic acid (Nip)
XLXQXLAELLENLLEXFLDLVX

SEQ ID NO: 163 - protein, 22 aa, Artificial Sequence
  Synthetic Construct
  MISC_FEATURE (22)..(22): Xaa is the non-natural amino acid nipecotic acid (Nip)
KLKQKLFELLENLLERFLDLVX

SEQ ID NO: 164 - protein, 22 aa, Artificial Sequence
  Synthetic Construct
  MISC_FEATURE (22)..(22): Xaa is the non-natural amino acid nipecotic acid (Nip)
KLKQRLADLLENLLEKFLELVX

SEQ ID NO: 165 - protein, 22 aa, Artificial Sequence
  Synthetic Construct
  MISC_FEATURE (22)..(22): Xaa is the non-natural amino acid nipecotic acid (Nip)
KLKQKLAELLEQLLDKFLELAX

SEQ ID NO: 166 - RNA, 69 nt, Homo sapiens
cuguggugcauuguaguugcauugcauguucuggugguacccaugcaauguuuccacagugcaucacag

SEQ ID NO: 167 - RNA, 21 nt, Homo sapiens
gugcauuguaguugcauugca

SEQ ID NO: 168 - RNA, 22 nt, Homo sapiens
caauguuuccacagugcaucac

SEQ ID NO: 169 - RNA, 96 nt, Homo sapiens
gcgggcggccccgcggugcauugcuguugcauugcacgugugugaggcgggugcagugccucggcagugcagcccggagccggccccuggcaccac

SEQ ID NO: 170 - RNA, 20 nt, Homo sapiens
gugcauugcuguugcauugc

SEQ ID NO: 171 - RNA, 22 nt, Homo sapiens
cagugccucggcagugcagccc

SEQ ID NO: 172 - RNA, 69 nt, Mus musculus
cuguggugcauuguaguugcauugcauguucuggcaauaccugugcaauguuuccacagugcaucacgg

SEQ ID NO: 173 - RNA, 21 nt, Mus musculus
gugcauuguaguugcauugca

SEQ ID NO: 174 - RNA, 22 nt, Mus musculus
caauguuuccacagugcaucac